(19) United States
(12) Patent Application Publication
(10) Pub. No.: US 2003/0232756 A1
(43) Pub. Date: Dec. 18, 2003

(54) Ligands for G protein coupled receptor GPR7 and GPR8 and uses thereof

(75) Inventors: Stephane Brezillion, Brussels (BE); Vincent Lannoy, Liernu (BE); Vincent Dupriez, Bruxelles (BE); Jean-Denis Franssen, Nivelles (BE); Michel Detheux, Mons (BE); Marc Parmentier, Beersel (BE); Emmanuel Le Poul, Bruxelles (BE)

Correspondence Address: PALMER & DODGE, LLP, KATHLEEN M. WILLIAMS, 111 HUNTINGTON AVENUE, BOSTON, MA, 02199, US

(73) Assignee: Euroscreen, S.A.

(21) Appl. No.: 10/403,408

(22) Filed: Mar. 31, 2003

(30) Foreign Application Priority Data
Mar. 29, 2002 (EP) 02447054.4
Aug. 22, 2002 (EP) 02447161.7

(51) Int. Cl. A61K 38/16; C07K 14/00; G01N 33/53

(52) U.S. Cl. 514/12; 530/324; 435/7.1

(57) ABSTRACT

The present invention is related to the identification of a family of polypeptide ligands that bind both the GPR7 and GPR8 G-protein coupled receptors, and to their use in screening methods for the identification of modulators of GPR7/8 receptor function and for diagnostic and therapeutic applications. The invention further relates to kits which use these orphan G protein coupled receptors and their ligands to identify agonist and antagonist compounds applicable to the diagnosis, prevention and/or treatment of diseases and disorders related to GPR7/8 receptor function or dysfunction.

### RELATED APPLICATIONS

This application claims the benefit under 35 U.S.C. §119 of EP Application No. 02447054.4, filed Mar. 29, 2002 and EP Application No. 02447161.7, filed Aug. 22, 2002, each of which is incorporated herein by reference in its entirety, including figures.

### FIELD OF THE INVENTION

The present invention is related to the natural ligand for an orphan G protein coupled receptor and methods of use.

### BACKGROUND OF THE INVENTION AND STATE OF THE ART

G-protein coupled receptors (GPCRs) are proteins responsible for transducing a signal within a cell. GPCRs have usually seven transmembrane domains. Upon binding of a ligand to an extra-cellular portion or fragment of a GPCR, a signal is transduced within the cell that results in a change in a biological or physiological property or behaviour of the cell. GPCRs, along with G-proteins and effectors (intracellular enzymes and channels modulated by G-proteins), are the components of a modular signalling system that connects the state of intra-cellular second messengers to extra-cellular inputs.

GPCR genes and gene products can modulate various physiological processes and are potential causative agents of disease. The GPCRs seem to be of critical importance to both the central nervous system and peripheral physiological processes.

The GPCR protein superfamily is represented in five families: Family I, receptors typified by rhodopsin and the beta2-adrenergic receptor and currently represented by over 200 unique members; Family II, the parathyroid hormone/calcitonin/secretin receptor family; Family III, the metabotropic glutamate receptor family, Family IV, the CAMP receptor family, important in the chemotaxis and development of D. discoideum; and Family V, the fungal mating pheromone receptor such as STE2.

G proteins represent a family of heterotrimeric proteins composed of α, β and γ subunits, that bind guanine nucleotides. These proteins are usually linked to cell surface receptors (receptors containing seven transmembrane domains) for signal transduction. Indeed, following ligand binding to the GPCR, a conformational change is transmitted to the G protein, which causes the α-subunit to exchange a bound GDP molecule for a GTP molecule and to dissociate from the βγ-subunits.

The GTP-bound form of the α, β and γ-subunits typically functions as an effector-modulating moiety, leading to the production of second messengers, such as cAMP (e.g. by activation of adenyl cyclase), diacylglycerol or inositol phosphates.

More than 20 different types of α-subunits are known in humans. These subunits associate with a small pool of β and γ subunits. Examples of mammalian G proteins include Gi, Go, Gq, Gs and Gt. G proteins are described extensively in Lodish et al., Molecular Cell Biology (Scientific American Books Inc., New York, N.Y., 1995; and also by Downes and Gautam, 1999, The G-Protein Subunit Gene Families. Genomics 62:544-552), the contents of both of which are incorporated herein by reference.

Known and uncharacterized GPCRs currently constitute major targets for drug action and development. There are ongoing efforts to identify new G protein coupled receptors which can be used to screen for new agonists and antagonists having potential prophylactic and therapeutical properties.

More than 300 GPCRs have been cloned to date, excluding the family of olfactory receptors. Mechanistically, approximately 50-60% of all clinically relevant drugs act by modulating the functions of various GPCRs (Cudermann et al., J. Mol. Med., 73:51-63, 1995).

GPR7 is a member of the rhodopsin like receptors family, cloned in 1995 (O'Dowd et al., 1995). Using oligonucleotides based on the opioid and somatostatin receptors, two novel G protein-coupled receptor genes were cloned starting from genomic DNA. The intronless coding sequences of these genes, named GPR7 and GPR8, shared 70% identity with each other, and each shared significant similarity with the sequences encoding transmembrane regions of the opioid and somatostatin receptors. GPR7 was mapped to chromosome 10q11.2-q21.1 and GPR8 to chromosome 20q13.3. Northern blot analysis using human mRNA demonstrated expression of GPR7 mainly in cerebellum and frontal cortex, while GPR8 was located mainly in the frontal cortex. In situ hybridization revealed expression of GPR7 in the human pituitary. A partial sequence of the mouse orthologue of GPR7 was obtained, and in situ hybridization demonstrated expression in discrete nuclei of brain, namely suprachiasmatic, arcuate, and ventromedial nuclei of hypothalamus. In situ hybridization analyses of rat brain was also performed and revealed specific patterns of expression in the brain. GPR7 mRNA was found to be discretely localized in areas of the amygdala, hippocampus, hypothalamus and cortex (Lee D. K. et al.; 1999). mRNA GPR7 is also found in Schwann cells and its expression is increased in patients with painful peripheral neuropathies with an inflammatory, immune and vasculitic etiology. Similar changes in GPR7 mRNA expression were observed in animal models of painful inflammatory peripheral neuropathies. Altered GPR7 expression in Schwann cells is hypothesized to disrupt myelination leading to progression of the neuropathy and/or axonal dysfunction leading to a painful phenotype. In addition, GPR7 may be specifically regulated during nerve repair processes and its increase of expression may contribute to trigger the phenotypic changes of sensory neurones that underlie neuropathic pain.

The aim of the present invention is to identify and isolate agents that modulate the activity of GPR7/8 receptors, using assays comprising GPR78 receptors, functional portions thereof and/or homologues thereof, and ligands or functional portions thereof and/or homologues thereof, the ligands having been found to bind to the receptors.

### SUMMARY OF THE INVENTION

The present invention relates to the discovery that members of the GPR7/8 family of G protein-coupled receptors bind a family of ligands comprising a consensus polypeptide sequence of WYKxxAxxxxxT/SVGRAAGLLxGL (“x” can be any amino acid). The invention further relates to the identification of members of the GPR7/8 ligand family including polypeptide ligands comprising or consisting of a sequence corresponding to SEQ ID NO: 3, SEQ ID NO: 5, SEQ ID NO: 7, SEQ ID NO: 9, SEQ ID NO: 11, SEQ ID NO: 15, SEQ ID NO: 17, SEQ ID NO: 19, SEQ ID NO: 21, SEQ ID NO: 25, SEQ ID NO: 27, as represented in FIG. 3, a homologous sequence thereof and/or a functional portion thereof, hereinafter, known as “group A”.

The present invention relates to a polypeptide ligand comprising or consisting of a sequence corresponding to SEQ ID NO: 3, SEQ ID NO: 5, SEQ ID NO: 7, SEQ ID NO: 9, SEQ ID NO: 11, SEQ ID NO: 13, SEQ ID NO: 19, SEQ ID NO: 25, SEQ ID NO: 27, as represented in FIG. 3, a homologous sequence thereof and/or a functional portion thereof, hereinafter, known as “group B”.

The present invention further relates to a polypeptide ligand comprising or consisting of a sequence corresponding to SEQ ID NO: 7, SEQ ID NO: 11, SEQ ID NO: 15, SEQ ID NO: 17, SEQ ID NO: 21, as represented in FIG. 3, a homologous sequence thereof and/or a portion thereof, hereinafter, known as “group C”. The peptides in this group are recited in Examples 4, 5, 6, 8 and 9.

The present invention further relates to a polypeptide ligand comprising or consisting of a sequence corresponding to SEQ ID NO: 11 as represented in FIG. 3, a homologous sequence thereof and/or a portion thereof, hereinafter, known as “group D”. The peptide in this group is recited in Examples 8 and 9.

The present invention further relates to a polypeptide ligand comprising or consisting of a sequence corresponding to SEQ ID NO: 7 and SEQ ID NO: 11 as represented in FIG. 3, a homologous sequence thereof and/ or a portion thereof, hereinafter, known as “group E”.

One embodiment of the present invention is a method of identifying an agent that modulates the function of a G-protein coupled receptor 7 (GPR7), said method comprising:

a) contacting a GPR7 polypeptide with a polypeptide ligand from group A, B or C in the presence and absence of a candidate modulator under conditions permitting the binding of said ligand to said GPR7 polypeptide; and

b) measuring the binding of said GPR7 polypeptide to said ligand, wherein a decrease in binding in the presence of said candidate modulator, relative to the binding in the absence of said candidate modulator, identifies said candidate modulator as an agent that modulates the function of GPR7.

Another embodiment of the present invention is a method of identifying an agent that modulates the function of a G-protein coupled receptor 8 (GPR8), said method comprising:

a) contacting a GPR8 polypeptide with a polypeptide ligand of from groups B or D in the presence and absence of a candidate modulator under conditions permitting the binding of said ligand to said GPR8 polypeptide; and

b) measuring the binding of said GPR8 polypeptide to said ligand, wherein a decrease in binding in the presence of said candidate modulator, relative to the binding in the absence of said candidate modulator, identifies said candidate modulator as an agent that modulates the function of GPR8.

Another embodiment of the present invention is a method of detecting the presence in a sample of an agent in a sample that modulates the function of GPR7, said method comprising:

a) contacting a GPR7 polypeptide with a polypeptide ligand from group A, B or C in the presence and absence of said sample under conditions permitting the binding of said ligand to said GPR7 polypeptide; and

b) measuring the binding of said GPR7 polypeptide to said ligand, wherein a decrease in binding in the presence of said sample, relative to the binding in the absence of said candidate modulator, identifies said candidate modulator as an agent that modulates the function of GPR7.

Another embodiment of the present invention is a method of detecting the presence in a sample of an agent in a sample that modulates the function of GPR8, said method comprising:

a) contacting a GPR8 polypeptide with a polypeptide ligand of groups B or D in the presence and absence of said sample under conditions permitting the binding of said ligand to said GPR8 polypeptide; and

b) measuring the binding of said GPR8 polypeptide to said ligand, wherein a decrease in binding in the presence of said sample, relative to the binding in the absence of said candidate modulator, identifies said candidate modulator as an agent that modulates the function of GPR8.

Another embodiment of the present invention is a method of identifying an agent that modulates the function of GPR7, said method comprising:

a) contacting a GPR7 polypeptide with a polypeptide ligand from group A, B or C in the presence and absence of a candidate modulator; and

b) measuring a signalling activity of said GPR7 polypeptide, wherein a change in the activity in the presence of said candidate modulator relative to the activity in the absence of said candidate modulator identifies said candidate modulator as an agent that modulates the function of GPR7.

Another embodiment of the present invention is a method of identifying an agent that modulates the function of GPR8, said method comprising:

a) contacting a GPR8 polypeptide with a polypeptide ligand of groups B or D in the presence and absence of a candidate modulator; and

b) measuring a signalling activity of said GPR8 polypeptide, wherein a change in the activity in the presence of said candidate modulator relative to the activity in the absence of said candidate modulator identifies said candidate modulator as an agent that modulates the function of GPR8.

Another embodiment of the present invention is a method of identifying an agent that modulates the function of GPR7, said method comprising:

a) contacting a GPR7 polypeptide with a candidate modulator;

b) measuring a signalling activity of said GPR7 polypeptide in the presence of said candidate modulator; and

c) comparing said activity measured in the presence of said candidate modulator to said activity measured in a sample in which said GPR7 polypeptide is contacted with a polypeptide ligand from group A, B or C at its EC₅₀, wherein said candidate modulator is identified as an agent that modulates the function of GPR7 when the amount of said activity measured in the presence of said candidate modulator is at least 20% of the amount induced by said ligand present at its EC₅₀.

Another embodiment of the present invention is a method of identifying an agent that modulates the function of GPR8, said method comprising:

a) contacting a GPR8 polypeptide with a candidate modulator;

b) measuring a signalling activity of said GPR8 polypeptide in the presence of said candidate modulator; and

c) comparing said activity measured in the presence of said candidate modulator to said activity measured in a sample in which said GPR8 polypeptide is contacted with a polypeptide ligand of groups B or D at its EC₅₀, wherein said candidate modulator is identified as an agent that modulates the function of GPR8 when the amount of said activity measured in the presence of said candidate modulator is at least 20% of the amount induced by said ligand present at its EC₅₀.

Another embodiment of the present invention is a method of detecting the presence, in a sample, of an agent that modulates the function of GPR7, said method comprising:

a) contacting a GPR7 polypeptide with a polypeptide from group A, B or C in the presence and absence of said sample;

b) measuring a signalling activity of said GPR7 polypeptide; and

c) comparing the amount of said activity measured in a reaction containing said GPR7 polypeptide and said ligand without said sample to the amount of said activity measured in a reaction containing said GPR7 polypeptide, said ligand and said sample, wherein a change in said activity in the presence of said sample relative to the activity in the absence of said sample indicates the presence, in said sample, of an agent that modulates the function of GPR7.

Another embodiment of the present invention is a method of detecting the presence, in a sample, of an agent that modulates the function of GPR8, said method comprising:

a) contacting a GPR8 polypeptide with a polypeptide ligand of groups B or D above in the presence and absence of said sample;

b) measuring a signalling activity of said GPR8 polypeptide; and

c) comparing the amount of said activity measured in a reaction containing said GPR8 polypeptide and said ligand without said sample to the amount of said activity measured in a reaction containing said GPR8 polypeptide, said ligand and said sample, wherein a change in said activity in the presence of said sample relative to the activity in the absence of said sample indicates the presence, in said sample, of an agent that modulates the function of GPR8.

Another embodiment of the present invention is a method of detecting the presence, in a sample, of an agent that modulates the function of GPR7, said method comprising:

a) contacting a GPR7 polypeptide with said sample;

b) measuring a signalling activity of said GPR7 polypeptide in the presence of said sample; and

c) comparing said activity measured in the presence of said sample to said activity measured in a reaction in which said GPR7 polypeptide is contacted with a polypeptide ligand from groups A, B or C present at its EC₅₀, wherein an agent that modulates the function of GPR7 is detected if the amount of said activity measured in the presence of said sample is at least 20% of the amount induced by said ligand present at its EC₅₀.

Another embodiment of the present invention is a method of detecting the presence, in a sample, of an agent that modulates the function of GPR8, said method comprising:

a) contacting a GPR8 polypeptide with said sample;

b) measuring a signalling activity of said GPR8 polypeptide in the presence of said sample; and

c) comparing said activity measured in the presence of said sample to said activity measured in a reaction in which said GPR8 polypeptide is contacted with a polypeptide ligand of groups B or D above present at its EC₅₀, wherein an agent that modulates the function of GPR8 is detected if the amount of said activity measured in the presence of said sample is at least 20% of the amount induced by said ligand present at its EC₅₀.

Another embodiment of the present invention is a method as defined above wherein said ligand is detectably labelled.

Another embodiment of the present invention is a method as defined above wherein said ligand is detectably labelled, said label is a moiety selected from the group consisting of a radioisotope, a fluorophore, a quencher of fluorescence, an enzyme.

Another embodiment of the present invention is a method as defined above wherein said contacting is performed in or on a cell expressing said GPR7 polypeptide.

Another embodiment of the present invention is a method as defined above wherein said contacting is performed in or on a cell expressing said GPR8 polypeptide.

Another embodiment of the present invention is a method as defined above wherein said contacting is performed in or on synthetic liposomes (Mirzabekov et al., 2000).

Another embodiment of the present invention is a method as defined above wherein said contacting is performed in or on virus-induced budding membranes containing a GPR7 polypeptide. (See Patent application WO0102551, Virus-like particles, their Preparation and their Use preferably in Pharmaceutical Screening and Functional Genomics (2001) incorporated herein by reference).

Another embodiment of the present invention is a method as defined above wherein said contacting is performed in or on virus-induced budding membranes containing a GPR8 polypeptide. (See Patent application WO0102551, Virus-like particles, their Preparation and their Use preferably in Pharmaceutical Screening and Functional Genomics (2001) incorporated herein by reference).

Another embodiment of the present invention is a method as defined above wherein said GPR7 polypeptide is expressed by cells and is present in a membrane fraction of said cells.

Another embodiment of the present invention is a method as defined above wherein said GPR8 polypeptide is expressed by cells and is present as a membrane fraction of said cells.

Preferably, the said cells are recombinant cells transformed by a plasmid or viral vector, preferably a baculovirus, an adenovirus, a semliki forest virus, and the cell is preferably selected from the group consisting of bacterial cells, yeast cells, insect cells or mammal cells.

According to a preferred embodiment of the present invention, the said cell is selected from the group consisting of COS-7 cells, a CHO—K1 cell, a LM (TK−) cell, a NIH-3T3 cell, HEK-293 cell, or K-562 cell but also other transfectable cell lines. Preferably, the vector comprises all the regulatory elements, operatively linked to the polynucleotide sequence encoding the receptor according to the invention so as to permit expression thereof.

Another embodiment of the present invention is a method as defined above wherein said measuring is performed using a method selected from label displacement, surface plasmon resonance, fluorescence resonance energy transfer, fluorescence quenching, and fluorescence polarization.

Another embodiment of the present invention is a method as defined above wherein said agent is selected from the group consisting of a peptide, a polypeptide, an antibody or antigen-binding fragment thereof, a lipid, a carbohydrate, a nucleic acid, a peptide-nucleic acid, and a small organic molecule.

Another embodiment of the present invention is a method as defined above wherein said step of measuring a signalling activity of said GPR7 polypeptide comprises detecting a change in the level of a second messenger.

Another embodiment of the present invention is a method as defined above wherein said step of measuring a signalling activity of said GPR8 polypeptide comprises detecting a change in the level of a second messenger.

Another embodiment of the present invention is a method as defined above wherein said measuring comprises measurement of guanine nucleotide binding or exchange, adenylate cyclase activity, cAMP, Protein Kinase C activity, phosphatidylinositol breakdown, diacylglycerol, inositol triphosphate, intracellular calcium, arachinoid acid, MAP kinase activity, tyrosine kinase activity, or reporter gene expression.

Another embodiment of the present invention is a method as defined above wherein said measuring comprises using an aequorin-based assay.

Another embodiment of the present invention is a kit for screening for agents that modulate the binding properties of GPR7 according to the methods as defined above.

Another embodiment of the present invention is a kit for screening for agents that modulate the binding properties of GPR8 according to the methods as defined above.

Another embodiment of the present invention is a kit for screening for agents that modulate the signalling activity of GPR7 according to the methods as defined above.

Another embodiment of the present invention is a kit for screening for agents that modulate the signalling activity of GPR8 according to the methods as defined above.

Another embodiment of the present invention comprises any of the kits as defined above comprising an isolated GPR7 polypeptide and/or a polypeptide ligand from group A, B or C.

Another embodiment of the present invention comprises any of the kits as defined above comprising an isolated GPR8 polypeptide and/or a polypeptide ligand of groups B or D.

Another embodiment of the present invention comprises any of the kits as defined above, said kit comprising an isolated polynucleotide encoding a GPR7 polypeptide.

Another embodiment of the present invention comprises any of the kits as defined above, said kit comprising an isolated polynucleotide encoding a GPR8 polypeptide.

Another embodiment of the present invention comprises any of the kits as defined above, said kit comprising cells transformed with a polynucleotide encoding a GPR7 polypeptide.

Another embodiment of the present invention comprises any of the kits as defined above, said kit comprising cells transformed with a polynucleotide encoding a GPR8 polypeptide.

Another embodiment of the present invention comprises any of the kits as defined above comprising a containing said GPR7 polypeptide, polynucleotide or transformed cells in a high-throughput screening kit format.

Another embodiment of the present invention comprises any of the kits as defined above comprising a containing said GPR8 polypeptide, polynucleotide or transformed cells in a high-throughput screening kit format.

Any of the kits as defined above, wherein kit comprises packaging materials therefor.

Another embodiment of the present invention a method of diagnosing a disease or disorder characterized by dysregulation of GPR7 signalling, said method comprising:

a) contacting a tissue sample with an antibody specific for a portion of a GPR7 polypeptide, said GPR7 polypeptide capable of associating with a polypeptide ligand from group A, B or C;

b) detecting binding of said antibody to said tissue sample; and

c) comparing the binding detected in step (b) with a standard, wherein a difference in binding relative to said standard is diagnostic of a disease or disorder characterized by dysregulation of GPR7.

Another embodiment of the present invention a method of diagnosing a disease or disorder characterized by dysregulation of GPR8 signalling, said method comprising:

a) contacting a tissue sample with an antibody specific for a portion of a GPR8 polypeptide, said GPR8 polypeptide capable of associating with a polypeptide ligand of groups B or D;

b) detecting binding of said antibody to said tissue sample; and

c) comparing the binding detected in step (b) with a standard, wherein a difference in binding relative to said standard is diagnostic of a disease or disorder characterized by dysregulation of GPR8.

Another embodiment of the present invention a method of diagnosing a disease or disorder characterized by dysregulation of GPR7 signalling, said method comprising:

a) isolating nucleic acid from a tissue sample;

b) amplifying a GPR7 polynucleotide encoding a portion of a GPR7 polypeptide said GPR7 polypeptide capable of associating with a polypeptide ligand from group A, B or C, amplification using said nucleic acid as a template; and

c) comparing the amount of amplified GPR7 polynucleotide produced in step (b) with a standard, wherein a difference in said amount of amplified GPR7 polynucleotide relative to said standard is diagnostic of a disease or disorder characterized by dysregulation of GPR7.

Another embodiment of the present invention a method of diagnosing a disease or disorder characterized by dysregulation of GPR8 signalling, said method comprising:

a) isolating nucleic acid from a tissue sample;

b) amplifying a GPR8 polynucleotide encoding a portion of a GPR8 polypeptide said GPR8 polypeptide capable of associating with a polypeptide ligand of groups A, B or D above, amplification using said nucleic acid as a template; and

c) comparing the amount of amplified GPR8 polynucleotide produced in step (b) with a standard, wherein a difference in said amount of amplified GPR8 polynucleotide relative to said standard is diagnostic of a disease or disorder characterized by dysregulation of GPR8.

Another embodiment of the present invention a method of diagnosing a disease or disorder characterized by dysregulation of GPR7 signalling, said method comprising:

a) isolating nucleic acid from a tissue sample;

b) amplifying a GPR7 polynucleotide encoding a portion of a GPR7 polypeptide, said GPR7 polypeptide capable of associating with a polypeptide ligand from group A, B or C, said amplification using said nucleic acid as a template; and

c) comparing the sequence of said amplified GPR7 polynucleotide produced in step (b) with a standard, wherein a difference in said sequence, relative to said standard is diagnostic of a disease or disorder characterized by dysregulation of GPR7.

Another embodiment of the present invention is a method as defined above wherein said standard is SEQ ID NO: 24.

Another embodiment of the present invention a method of diagnosing a disease or disorder characterized by dysregulation of GPR8 signalling, said method comprising:

a) isolating nucleic acid from a tissue sample;

b) amplifying a GPR8 polynucleotide encoding a portion of a GPR8 polypeptide, said GPR8 polypeptide capable of associating with a polypeptide ligand of groups B or D above, said amplification using said nucleic acid as a template; and

c) comparing the sequence of said amplified GPR8 polynucleotide produced in step (b) with a standard, wherein a difference in said sequence, relative to said standard is diagnostic of a disease or disorder characterized by dysregulation of GPR8.

Another embodiment of the present invention is a method as defined above wherein said standard is GPR8.

Another embodiment of the present invention is a method as defined above wherein said standard is SEQ ID NO: 30.

Another embodiment of the present invention is a method as defined above wherein said comparing of the sequence is performed on a microarray.

Another embodiment of the present invention a kit for the diagnosis of a disease or disorder characterized by dysregulation of GPR7 signalling suitable for carrying out any of the methods of diagnosing a disease or disorder characterized by dysregulation as defined above.

Another embodiment of the present invention a kit for the diagnosis of a disease or disorder characterized by dysregulation of GPR8 signalling suitable for carrying out any of the methods of diagnosing a disease or disorder characterized by dysregulation as defined above.

Another embodiment of the present invention comprises any of the kits as defined above comprising an isolated GPR7 polypeptide and/or a polypeptide ligand from group A, B or C.

Another embodiment of the present invention comprises any of the kits as defined above comprising an isolated GPR8 polypeptide and/or a polypeptide ligand of groups B or D.

Another embodiment of the present invention comprises any of the kits as defined above comprising an isolated polynucleotide encoding a GPR7 polypeptide.

Another embodiment of the present invention comprises any of the kits as defined above comprising an isolated polynucleotide encoding a GPR8 polypeptide.

Another embodiment of the present invention comprises any of the kits as defined above, comprising a cell transformed with a polynucleotide encoding a GPR7 polypeptide.

Another embodiment of the present invention comprises any of the kits as defined above, comprising a cell transformed with a polynucleotide encoding a GPR8 polypeptide.

Another embodiment of the present invention comprises any of the kits as defined above, wherein kit comprises packaging materials therefor.

Another embodiment of the present invention is an unknown agent that modulates the binding property between a GPR7 polypeptide and a polypeptide ligand from group A, B or C, which is obtainable by using the method for identifying an agent that modulates the function of GPR7 according to the methods as defined above.

Another embodiment of the present invention is an unknown agent that modulates the binding property between a GPR8 polypeptide and a polypeptide ligand of groups B or D above, which is obtainable by using the method for identifying an agent that modulates the function of GPR8 according to the methods as defined above.

Another embodiment of the present invention is an unknown agent that modulates the signalling activity between a GPR7 polypeptide and a polypeptide ligand from group A, B or C, which is obtainable by using the method for identifying an agent that modulates the function of GPR7 according to the methods as defined above.

Another embodiment of the present invention is an unknown agent that modulates the signalling activity between a GPR8 polypeptide and a polypeptide ligand of groups B or D, which is obtainable by using the method for identifying an agent that modulates the function of GPR8 according to the methods as defined above.

Another embodiment of the present invention is an agent as defined above for use as a medicament.

Another embodiment of the present invention is an agent as defined above for the manufacture of a medicament for the preventing, treating and/or alleviating diseases caused by GPR7 receptor misfunction, such as diseases or disorders selected from the group consisting of ostatic hypertrophy, migraine, vomiting, psychotic and neurological disorders, including anxiety, schizophrenia, manic depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome and other related diseases including thrombosis and other cardiovascular diseases, autoimmune and inflammatory diseases, fertility, fetal development, infections such as bacterial, fungal, protozoan and viral infections, particularly infections caused by HIV1 and HIV2, pain, cancer, anorexia, bulimia, asthma, Parkinson's disease, acute heart failure, hypertension, urinary retention, osteoporosis, angina pectoris, myocardial infarction, ulcers, asthma, allergies, benign prostatic hypertrophy, stroke, disturbances of cell migration, cancer, development of tumours and tumour metastasis, inflammatory and neo-plastic processes, wound and bone healing and dysfunction of regulatory growth functions, diabetes, obesity, anorexia, bulimia, acute heart failure, hypotension, hypertension, urinary retention, osteoporosis, angina pectoris, myocardial infarction, restenosis, atherosclerosis, thrombosis and other cardiovascular diseases, autoimmune and inflammatory diseases, diseases characterized by excessive smooth muscle cell proliferation, aneurysms, diseases characterized by loss of smooth muscle cells or reduced smooth muscle cell proliferation, stroke, ischemia, ulcers, allergies, benign prostatic hypertrophy, migraine, vomiting, psychotic and neurological disorders, including anxiety, schizophrenia, manic depression, depression, delirium, dementia and severe mental retardation, degenerative diseases, and other cardiovascular diseases, autoimmune and inflammatory diseases, or disorders or diseases related to any of the following organs : cerebellum, frontal cortex, hypothalamus, pituitary gland, amygdala, brain, spinal cord, liver, testis, colon, trachea, rectum and small intestine.

Another embodiment of the present invention is an agent as defined above for the manufacture of a medicament for the preventing, treating and/or alleviating diseases caused by GPR8 receptor misfunction, such as diseases or disorders selected from the group consisting of ostatic hypertrophy, migraine, vomiting, psychotic and neurological disorders, including anxiety, schizophrenia, manic depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome and other related diseases including thrombosis and other cardiovascular diseases, autoimmune and inflammatory diseases, fertility, fetal development, infections such as bacterial, fungal, protozoan and viral-infections, particularly infections caused by HIV1 and HIV2, pain, cancer, anorexia, bulimia, asthma, Parkinson's disease, acute heart failure, hypertension, urinary retention, osteoporosis, angina pectoris, myocardial infarction, ulcers, asthma, allergies, benign prostatic hypertrophy, stroke, disturbances of cell migration, cancer, development of tumours and tumour metastasis, inflammatory and -neo-plastic processes, wound and bone healing and dysfunction of regulatory growth functions, diabetes, obesity, anorexia, bulimia, acute heart failure, hypotension, hypertension, urinary retention, osteoporosis, angina pectoris, myocardial infarction, restenosis, atherosclerosis, thrombosis and other cardiovascular diseases, autoimmune and inflammatory diseases, diseases characterized by excessive smooth muscle cell proliferation, aneurysms, diseases characterized by loss of smooth muscle cells or reduced smooth muscle cell proliferation, stroke, ischemia, ulcers, allergies, benign prostatic hypertrophy, migraine, vomiting, psychotic and neurological disorders, including anxiety, schizophrenia, manic depression, depression, delirium, dementia and severe mental retardation, degenerative diseases, and other cardiovascular diseases, autoimmune and inflammatory diseases, or disorders or diseases related to any of the following organs: cerebellum, frontal cortex, hypothalamus, pituitary gland, amygdala, brain, spinal cord, liver, testis, colon, trachea, rectum and small intestine.

Another embodiment of the present invention is a method for the production of a composition comprising the steps of admixing the agent as defined above with a pharmaceutically acceptable carrier.

Another embodiment of the present invention is a composition comprising the agent as defined above.

Another embodiment of the present invention is any of the methods as defined above wherein said GPR7 polypeptide comprises the sequence corresponding to SEQ ID NO: 23, a homologue thereof, or a functional portion thereof.

Another embodiment of the present invention is any of the methods as defined above wherein said GPR8 polypeptide comprises the sequence corresponding to SEQ ID NO: 29, a homologue thereof, or a functional portion thereof.

Another embodiment of the present invention is any of the kits as defined above wherein said GPR7 polypeptide comprises the sequence corresponding to SEQ ID NO: 23, a homologue thereof, or a functional portion thereof.

Another embodiment of the present invention is any of the kits as defined above wherein said GPR8 polypeptide comprises the sequence corresponding to SEQ ID NO: 29, a homologue thereof, or a functional portion thereof.

Another embodiment of the present invention is a polypeptide comprising a functional portion of SEQ ID NO: 23, required to associate with any of the polypeptides comprising a sequence from group A or C a homologous sequence of and/or a functional portion of said SEQ ID NOs.

Another embodiment of the present invention is a polypeptide which is a homologue of the functional portion of SEQ ID NO: 23 as defined in the paragraph above.

Another embodiment of the present invention is a polypeptide which comprises an immunologically active fragment of the any of the polypeptides as defined above.

Another embodiment of the present invention is a polypeptide which is a homologue of the immunologically active fragment as in the paragraph defined above.

Another embodiment of the present invention is a nucleic acid capable of encoding a polypeptide ligand of groups A and C and any of the polypeptides as defined in the above 4 paragraphs, said paragraphs concerning SEQ ID NO: 23 said nucleic acid hereinafter known as “group F”.

Another embodiment of the present invention is a nucleic acid comprising or consisting of a sequence corresponding to any of SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 26, SEQ ID NO: 28, as represented in FIG. 3, a homologous sequence of and/or a functional portion of said SEQ ID NOs, hereinafter, known as “group H”.

Another embodiment of the present invention is a nucleic acid comprising a sequence corresponding to any of SEQ ID NO: 8, SEQ ID NO: 12, SEQ ID NO: 16, SEQ ID NO: 18, SEQ ID NO: 22, as represented in FIG. 3, a homologous sequence of and/or a functional portion of said SEQ ID NOs, hereinafter, known as “group I”.

Another embodiment of the present invention is a nucleic acid comprising a sequence corresponding to any of SEQ ID NO: 4, SEQ ID NO: 6, SEQ ID NO: 8, as represented in FIG. 3, a homologous sequence of and/or a functional portion of said SEQ ID NOs, hereinafter, known as “group L”.

Another embodiment of the present invention is a nucleic acid of at least 15 nucleotides in length capable of specifically hybridising with the nucleic acid of groups F, H, J or L.

Another embodiment of the present invention is a nucleic acid molecule of at least 15 nucleotides in length, capable of specifically amplifying the nucleic acid as defined in the paragraph above.

Another embodiment of the present invention is a nucleic acid as defined above, derivatised, such that its hybrisation to underivatised complementary nucleic acid remains temperature dependent.

Another embodiment of the present invention is a nucleic acid of at least 15 nucleotides in length having the following characteristics:

i) capable of specifically hybridising with the nucleic acid as defined above, and

ii) derivatised, such its hybrisation to underivatised complementary nucleic acid remains temperature-dependent.

Another embodiment of the present invention is a vector comprising a nucleic acid sequence as defined above.

Another embodiment of the present invention is a vector as defined above wherein said vector is an expression vector wherein the nucleic acid sequence is operably linked to one or more control sequences allowing the expression of said sequence in prokaryotic and/or eukaryotic host cells.

Another embodiment of the present invention is any one of the vectors as defined above, wherein said vector is an adenoviral vector.

Another embodiment of the present invention is a host cell comprising an integrated or episomal copy of a nucleic acid molecule as defined above or any one of the vectors as defined above.

Another embodiment of the present invention is a host cell as defined above, wherein said host cell is a yeast, bacterial, insect, fungal, plant or mammalian cell.

Another embodiment of the present invention is an antibody specifically recognising any of the polypeptides as defined above.

Another embodiment of the present invention is an antibody specifically recognising a nucleic acid as defined above.

Another embodiment of the present invention is the use of an antibody as defined above to purify, detect, target and/or inhibit the activity of any of one of polypeptides as defined above.

Another embodiment of the present invention is the use of an antibody as defined above to purify, detect, target and/or inhibit the activity of a nucleic acid as defined above.

Another embodiment of the present invention is a method of detecting a nucleic acid as defined above.

Another embodiment of the present invention is a method of detecting any of the polypeptides as defined above.

Another embodiment of the present invention is a method for producing any of the polypeptides as defined above comprising:

(a) culturing host cells comprising a nucleic acid as defined above, under conditions allowing the expression of the polypeptide, and,

(b) recovering the produced polypeptide from the culture.

Another embodiment of the present invention is any of one of polypeptides as defined above for use as a medicament.

Another embodiment of the present invention is a nucleic acid as defined above for use as a medicament.

Another embodiment of the present invention is an antibody as defined above for use as a medicament.

Another embodiment of the present invention is the use of any of the polypeptides as defined above for preventing, treating and/or alleviating diseases caused by GPR7 receptor misfunction, or any other disease or disorder as defined above.

Another embodiment of the present invention is the use of a nucleic acid as defined above for preventing, treating and/or alleviating diseases caused by GPR7 receptor misfunction, or any other disease or disorder as defined above.

Another embodiment of the present invention is the use of an antibody as defined above for preventing, treating and/or alleviating diseases caused by GPR7 receptor misfunction, or any other disease or disorder as defined above.

Another embodiment of the present invention is the use of any of the polypeptides as defined above for the preparation of a medicament for preventing, treating and/or alleviating diseases caused by GPR7 receptor misfunction, or any other disease or disorder as defined above.

Another embodiment of the present invention is the use of a nucleic acid as defined above for the preparation of a medicament for preventing, treating and/or alleviating diseases caused by GPR7 receptor misfunction, or any other disease or disorder as defined above.

Another embodiment of the present invention is the use of an antibody as defined above for the preparation of a medicament for preventing, treating and/or alleviating diseases caused by GPR7 receptor misfunction, or any other disease or disorder as defined above.

Another embodiment of the present invention is a transgenic non-human animal comprising one or more copies of a nucleic acid as defined above stably integrated into the genome of said animal, or an animal comprising regulatory elements that modulate the expression of a nucleic acid as defined above.

Another embodiment of the present invention is a knock-out non-human animal comprising a deletion of one or more exons from one or two alleles encoding a nucleic acid as defined above, or the deletion of one or more exons of said nucleic acid, or an animal comprising a targeted mutation in the genomic region, including regulatory sequences, comprising any of the nucleic acid sequences as defined above.

Another embodiment of the present invention is the use of a transgenic or knock-out non-human animal as defined above as a model system.

Another embodiment of the present invention is a therapeutic composition comprising an agent as defined above.

Another embodiment of the present invention is the use of a therapeutic agent as defined above for the preparation of a medicament for treating a disease or disorder as defined above.

Another embodiment of the present invention is a polypeptide comprising a functional portion of SEQ ID NO: 29, required to associate with any of the polypeptides comprising a sequence from group B or D a homologous sequence of and/or a functional portion of said SEQ ID NOs.

Another embodiment of the present invention is a polypeptide which is a homologue of the functional portion of SEQ ID NO: 29 as defined in the paragraph above.

Another embodiment of the present invention is a polypeptide which comprises an immunologically active fragment of the any of the polypeptides as defined in the above 2 paragraphs or of groups B or D.

Another embodiment of the present invention is a polypeptide which is a homologue of the immunologically active fragment as in the paragraph defined above.

Another embodiment of the present invention is a nucleic acid capable of encoding a polypeptide ligand of groups B and D and any of the polypeptides as defined in the above 4 paragraphs, said paragraphs concerning SEQ ID NO: 29, said nucleic acid hereinafter known as “group G”.

Another embodiment of the present invention is a nucleic acid comprising or consisting of a sequence corresponding to any of SEQ ID NO: 4, SEQ ID NO: 6, SEQ ID NO: 8, SEQ ID NO: 10, SEQ ID NO: 12, SEQ ID NO: 20, SEQ ID NO: 26, SEQ ID NO: 28, as represented in FIG. 3, a homologous sequence of and/or a functional portion of said SEQ ID NOs, hereinafter, known as “group I”.

Another embodiment of the present invention is a nucleic acid comprising a sequence corresponding to SEQ ID NO: 12 as represented in FIG. 3, a homologous sequence of and/or a functional portion of said SEQ ID NO, hereinafter, known as “group K”.

Another embodiment of the present invention is a nucleic acid of at least 15 nucleotides in length capable of specifically hybridising with the nucleic acid of groups G, I, K or L.

Another embodiment of the present invention is a nucleic acid molecule of at least nucleotides in length, capable of specifically amplifying the nucleic acid as defined in the paragraph above.

Another embodiment of the present invention is a nucleic acid as defined in the paragraphs above, derivatised, such that its hybrisation to underivatised complementary nucleic acid remains temperature dependent.

Another embodiment of the present invention is a nucleic acid of at least 15 nucleotides in length having the following characteristics:

i) capable of specifically hybridising with the nucleic acid as defined in the paragraphs above, and

ii) derivatised, such its hybrisation to underivatised complementary nucleic acid remains temperature-dependent.

Another embodiment of the present invention is a vector comprising a nucleic acid sequence of groups G, I, K or L or variations thereof as defined above.

Another embodiment of the present invention is a vector as defined above wherein said vector is an expression vector wherein the nucleic acid sequence is operably linked to one or more control sequences allowing the expression of said sequence in prokaryotic and/or eukaryotic host cells.

Another embodiment of the present invention is any one of the vectors as defined above, wherein said vector is an adenoviral vector.

Another embodiment of the present invention is a host cell comprising an integrated or episomal copy of a nucleic acid molecule of groups G, I, K or L or variations thereof as defined above or any one of the vectors as defined above.

Another embodiment of the present invention is a host cell as defined above, wherein said host cell is a yeast, bacterial, insect, fungal, plant or mammalian cell.

Another embodiment of the present invention is an antibody specifically recognising any of the polypeptides of groups B and D, or functional portions of SEQ ID NO: 29 or variations thereof as defined above.

Another embodiment of the present invention is an antibody specifically recognising a nucleic acid of groups G, I, K or L or variations thereof as defined above.

Another embodiment of the present invention is the use of an antibody as defined above to purify, detect, target and/or inhibit the activity of the polypeptides of groups B and D or functional portions of SEQ ID NO: 29 or variations thereof as defined above.

Another embodiment of the present invention is the use of an antibody as defined above to purify, detect, target and/or inhibit the activity of a nucleic acid of groups G, I, K or L or variations thereof as defined above.

Another embodiment of the present invention is a method of detecting a nucleic acid of groups G, I, K or L or variations thereof as defined above.

Another embodiment of the present invention is a method of detecting any of the polypeptides of groups B and D or functional portions of SEQ ID NO: 29 or variations thereof as defined above.

Another embodiment of the present invention is a method for producing any of the polypeptides of groups B and D or functional portions of SEQ ID NO: 29 or variations thereof as defined above comprising:

(a) culturing host cells comprising a nucleic acid of groups G, I, K or L or variations thereof as defined above, under conditions allowing the expression of the polypeptide, and,

(b) recovering the produced polypeptide from the culture.

Another embodiment of the present invention is any of one of polypeptides of groups B and D or functional portions of SEQ ID NO: 29 or variations thereof as defined above for use as a medicament.

Another embodiment of the present invention is nucleic acid of groups G, I, K or L or variations thereof as defined above for use as a medicament.

Another embodiment of the present invention is an antibody as defined above for use as a medicament.

Another embodiment of the present invention is the use of any of the polypeptides of groups B and D or functional portions of SEQ ID NO: 29 or variations thereof as defined above for preventing, treating and/or alleviating diseases caused by GPR7 receptor misfunction, or any other disease or disorder as defined above.

Another embodiment of the present invention is the use of a nucleic acid of groups G, I, K or L or variations thereof as defined above for preventing, treating and/or alleviating diseases caused by GPR8 receptor misfunction, or any other disease or disorder as defined above.

Another embodiment of the present invention is the use of an antibody as defined above for preventing, treating and/or alleviating diseases caused by GPR8 receptor misfunction, or any other disease or disorder as defined above.

Another embodiment of the present invention is the use of any of the polypeptides of groups B and D or functional portions of SEQ ID NO: 29 or variations thereof as defined above for the preparation of a medicament for preventing, treating and/or alleviating diseases caused by GPR8 receptor misfunction, or any other disease or disorder as defined above.

Another embodiment of the present invention is the use of a nucleic acid of groups G, I, K or L or variations thereof as defined above for the preparation of a medicament for preventing, treating and/or alleviating diseases caused by GPR8 receptor misfunction, or any other disease or disorder as defined above.

Another embodiment of the present invention is the use of an antibody as defined above for the preparation of a medicament for preventing, treating and/or alleviating diseases caused by GPR8 receptor misfunction, or any other disease or disorder as defined above.

Another embodiment of the present invention is a transgenic non-human animal comprising one or more copies of a nucleic acid of groups G, I, K or L or variations thereof as defined above stably integrated into the genome of said animal, or an animal comprising regulatory elements that modulate the expression of a nucleic acid as defined above.

Another embodiment of the present invention is a knock-out non-human animal comprising a deletion of one or more exons from one or two alleles encoding a nucleic acid of groups G, I, K or L or variations thereof as defined above, or the deletion of one or more exons of said nucleic acid, or an animal comprising a targeted mutation in the genomic region, including regulatory sequences, comprising any of the nucleic acid sequences as defined above.

Another embodiment of the present invention is the use of a transgenic or knock-out non-human animal as defined above as a model system.

Another embodiment of the present invention is a therapeutic composition comprising an agent as defined above.

Another embodiment of the present invention is the use of a therapeutic agent as defined above for the preparation of a medicament for treating a disease or disorder as defined above.

Another embodiment of the present invention is a nucleic acid comprising a sequence corresponding to any of SEQ ID NO: 4, SEQ ID NO: 6, SEQ ID NO: 8, as represented in FIG. 3, a homologous sequence of and/or a functional portion of said SEQ ID NOs, hereinafter, known as “group L”.

Another embodiment of the present invention is a nucleic acid comprising a sequence corresponding to any of SEQ ID NO: 4, SEQ ID NO: 6, SEQ ID NO: 8, SEQ ID NO: 10, SEQ ID NO: 20 as represented in FIG. 3, a homologous sequence of and/or a functional portion of said SEQ ID NOs, hereinafter, known as “group M”.

Another embodiment of the present invention is a nucleic acid of at least 15 nucleotides in length capable of specifically hybridising with the nucleic acid of groups L or M.

Another embodiment of the present invention is a nucleic acid molecule of at least 15 nucleotides in length, capable of specifically amplifying the nucleic acid of groups L or M.

Another embodiment of the present invention is a nucleic acid as defined in the paragraphs above, derivatised, such that its hybrisation to underivatised complementary nucleic acid remains temperature dependent.

Another embodiment of the present invention is a nucleic acid as defined in the paragraphs above of at least 15 nucleotides in length having the following characteristics:

i) capable of specifically hybridising with the nucleic acid as defined above, and

ii) derivatised, such its hybrisation to underivatised complementary nucleic acid remains temperature-dependent.

Another embodiment of the present invention is a vector comprising a nucleic acid sequence of groups L or M or variations thereof as defined above.

Another embodiment of the present invention is a vector as defined above wherein said vector is an expression vector wherein the nucleic acid sequence is operably linked to one or more control sequences allowing the expression of said sequence in prokaryotic and/or eukaryotic host cells.

Another embodiment of the present invention is any one of the vectors as defined above, wherein said vector is an adenoviral vector.

Another embodiment of the present invention is a host cell comprising an integrated or episomal copy of a nucleic acid sequence of groups L or M or variations thereof as defined above or any one of the vectors as defined above.

Another embodiment of the present invention is a host cell as defined above, wherein said host cell is a yeast, bacterial, insect, fungal, plant or mammalian cell.

Another embodiment of the present invention is an antibody specifically recognising a nucleic acid sequence of groups L or M or variations thereof as defined above.

Another embodiment of the present invention is the use of an antibody as defined above to purify, detect, target and/or inhibit the activity of a nucleic acid sequence of groups L or M or variations thereof as defined above.

Another embodiment of the present invention is a method of detecting a nucleic acid sequence of groups L or M or variations thereof as defined above.

Another embodiment of the present invention is nucleic acid sequence of groups L or M or variations thereof as defined above for use as a medicament.

Another embodiment of the present invention is an antibody as defined above for use as a medicament.

Another embodiment of the present invention is the use of a nucleic acid sequence of groups L or M or variations thereof as defined above for preventing, treating and/or alleviating diseases caused by GPR7 receptor misfunction, or any other disease or disorder as defined above.

Another embodiment of the present invention is the use of a nucleic acid sequence of groups L or M or variations thereof as defined above for preventing, treating and/or alleviating diseases caused by GPR8 receptor misfunction, or any other disease or disorder as defined above.

Another embodiment of the present invention is the use of an antibody as defined above for preventing, treating and/or alleviating diseases caused by GPR7 receptor misfunction, or any other disease or disorder as defined above.

Another embodiment of the present invention is the use of an antibody as defined above for preventing, treating and/or alleviating diseases caused by GPR8 receptor misfunction, or any other disease or disorder as defined above.

Another embodiment of the present invention is the use of a nucleic acid sequence of groups L or M or variations thereof as defined above for the preparation of a medicament for preventing, treating and/or alleviating diseases caused by GPR7 receptor misfunction, or any other disease or disorder as defined above.

Another embodiment of the present invention is the use of a nucleic acid sequence of groups L or M or variations thereof as defined above for the preparation of a medicament for preventing, treating and/or alleviating diseases caused by GPR8 receptor misfunction, or any other disease or disorder as defined above.

Another embodiment of the present invention is the use of an antibody as defined above for the preparation of a medicament for preventing, treating and/or alleviating diseases caused by GPR7 receptor misfunction, or any other disease or disorder as defined above.

Another embodiment of the present invention is the use of an antibody as defined above for the preparation of a medicament for preventing, treating and/or alleviating diseases caused by GPR8 receptor misfunction, or any other disease or disorder as defined above.

Another embodiment of the present invention is a transgenic non-human animal comprising one or more copies of a nucleic acid sequence of groups L or M or variations thereof as defined above stably integrated into the genome of said animal, or an animal comprising regulatory elements that modulate the expression of a nucleic acid as defined above.

Another embodiment of the present invention is a knock-out non-human animal comprising a deletion of one or more exons from one or two alleles encoding a nucleic acid sequence of groups L or M or variations thereof as defined above, or the deletion of one or more exons of said nucleic acid, or an animal comprising a targeted mutation in the genomic region, including regulatory sequences, comprising any of the nucleic acid sequences as defined above.

Another embodiment of the present invention is the use of a transgenic or knock-out non-human animal as defined above as a model system.

### DETAILED DESCRIPTION OF THE INVENTION

The invention recognizes that a ligand family comprising the consensus amino acid sequence WYKxxAxxxxxT/SVGRAAGLLxGL binds orphan G protein coupled receptors GPR7, GPR8 and GPR7/8 receptor polypeptide family members. The invention also relates to members of that ligand family including L6L (SEQ ID NO: 3), L6C (SEQ ID NO: 5), L7′ (SEQ ID NO: 7), L7′L (SEQ ID NO: 9), L7 (SEQ ID NO: 11), L7C (SEQ ID NO: 15), L8 (SEQ ID NO: 17), L8L (SEQ ID NO: 19), L8C (SEQ ID NO: 11), 21) and ppL7 (SEQ ID: NO: 25), and ppL8 (SEQ ID NO: 27), as in FIG. 3 and to homologues thereof, each of which bind GPR7, GPR8 and GPR7/8 polypeptides. The invention further relates to methods of using the binding of these ligand to the receptor in a drug screening method. Said ligand and their interactions with the receptors GPR7, GPR8 and GPR7/8 polypeptides also provides for the diagnosis of conditions involving dysregulated receptor activity

The present invention is related to the “GPR7 receptor” defined by SEQ ID NO: 23, (encoded by nucleic acid SEQ ID NO: 24), and a recombinant cell (transformed by a suitable vector) comprising the nucleotide sequence encoding the receptor, as well as polypeptide ligands (e.g. L6L, L6C, L7′, L7′L, L7, L7C, L8L and L8C) and precursor polypeptide ligands (e.g. ppL7, ppL8), to be used in screening assays for identification of agonists, inverse agonists or antagonist compounds useful for the development of new drugs and the improvement of various disease diagnostics.

The invention also comtemplates homologues and active fragments of the GPR7 receptor that are equivalents of the receptor. Thus, a homologue has at least 62% amino acid sequence identity, preferably at least 70%, 80%, 85%, 90%, 95% or higher, up to and including 100% sequence identity with SEQ ID NO: 23. A GPR7 homologue polypeptide also has GPR7 activity, including either or both of GPR7 ligand binding activity or GPR7 signalling activity as defined herein. GPR7 ligand binding activity means binding of a ligand comprising a consensus polypeptide sequence of WYKxxAxxxxxT/SVGRAAGLLxGL (“x” can be any amino acid), or binding of at least one of the polypeptides represented by SEQ ID NOs: 3, 5, 7, 9, 11, 15, 17, 19, 21, 25 and 27. An “active fragment” of the GPR7 receptor exhibits GPR7 activityalso has GPR7 activity, including either or both of GPR7 ligand binding activity or GPR7 signalling activity as defined herein. Further, a naturally-occurring GPR7 polypeptide as the term is used herein is expressed in at least a subset of the tissues in which the polypeptide of SEQ ID NO: 23 is expressed, as shown in Table 1. As a non-limiting example, a “subset of tissues” can be central nervous system (CNS) and endocrine system, or, for example, the subset could be a further limited subset of a system, e.g., hippocampus and amygdala, which are a subset of the CNS.

The present invention is also related to the “GPR8 receptor” defined by SEQ ID NO: 29 (encoded by nucleic acid SEQ ID NO: 30), and a recombinant cell (transformed by a suitable vector) comprising the nucleotide sequence encoding the receptor, as well as polypeptide ligands (e.g. L6L, L6C, L7′, L7′L, L7, L8L and L8C) and precursor polypeptide ligands (e.g. ppL7, ppL8), to be used in screening assays for identification of agonists, inverse agonists or antagonist compounds useful for the development of new drugs and the improvement of various disease diagnostics.

The invention also comtemplates homologues and active fragments of the GPR8 receptor that are equivalents of the receptor. Thus, a homologue has at least 62% amino acid sequence identity, preferably at least 70%, 80%, 85%, 90%, 95% or higher, up to and including 100% sequence identity with SEQ ID NO: 29. A GPR8 homologue polypeptide also has GPR8 activity, including either or both of GPR8 ligand binding activity or GPR8 signalling activity as defined herein. GPR8 ligand binding activity means binding of a ligand comprising a consensus polypeptide sequence of WYKxxAxxxxxT/SVGRAAGLLxGL (“x” can be any amino acid), or binding of at least one of the polypeptides represented by SEQ ID NOs: 3, 5, 7, 9, 11, 15, 17, 19, 21, 25 and 27. An “active fragment” of the GPR8 receptor exhibits GPR8 activityalso has GPR8 activity, including either or both of GPR8 ligand binding activity or GPR8 signalling activity as defined herein. Further, a naturally-occurring GPR8 polypeptide as the term is used herein is expressed in at least a subset of the tissues in which the polypeptide of SEQ ID NO: 29 is expressed, as shown in Table 1. As a non-limiting example, a “subset of tissues” can be central nervous system (CNS) and endocrine system, or, for example, the subset could be a further limited subset of a system, e.g., hippocampus and amygdala, which are a subset of the CNS.

The naturally-occurring GPR7 and GPR8 polypeptides (SEQ ID NOs 23 and 29, respectively) bind a set of ligands having the same consensus sequence (WYKxxAxxxxxT/SVGRAAGLLxGL) and have 62% polypeptide sequence identity to each other over their full lengths. Thus, a “GPR7/8 receptor” homologue, by definition, has at least 62% sequence identity to either or both of SEQ ID NOs 23 and 29 over their full length. A GPR7/8 receptor homologue also has an activity of GPR7 and GPR8, including either or both of common ligand binding activity of the GPR7 and GPR8 receptors or a signalling activity of the GPR7 and GPR8 receptors as defined herein. There is even greater % identity over particular regions of the GPR7 and GPR8 polypeptides. The following shows the % identities over individual regions of the receptor molecules (TM=transmembrane region, I=intracellular loop, and E=extracellular loop):

| | |
|---|---|
| N-terminus: | 28% |
| TM1: | 60% |
| I1: | 100% |
| TM2: | 79% |
| E1: | 66% |
| TM3: | 70% |
| I2: | 62.5% |
| TM4: | 52% |
| E2: | 40% |
| TM5: | 73% |
| I3: | 65% |
| TM6: | 67% |
| E3: | 100% |
| TM7: | 87.5% |
| C-terminus: | 45% |

As shown herein, the nucleic acids of SEQ ID NOs 24 and 30, naturally encoding GPR7 and GPR8 polypeptides of SEQ ID NOs 23 and 29, respectively, have 71% sequence identity to each other. A “GPR7 polynucleotide” encodes a GPR7 polypeptide as the term is defined herein, or a functional portion of a GPR7 polypeptide as that term is defined herein. Similarly, a “GPR8 polynucleotide” encodes a GPR8 polypeptide as the term is defined herein, or a functional portion of a GPR8 polypeptide as that term is defined herein.

As used herein, a “functional portion” of a polypeptide, e.g., a receptor, refers to a partial polypeptide sequence that binds one or more ligands bound by the intact polypeptide comprising that sequence. The term “functional portion” also encompasses a partial polypeptide sequence of sufficient size to exhibit one or more signaling activities in response to binding of one or more ligands bound by the intact polypeptide comprising that sequence. As used herein, a “functional portion” or “functional fragment” of a nucleic acid sequence is one which encodes a functional portion of a polypeptide as that term is defined above.

“A portion” or “fragment” as it refers to a receptor sequence or a polypeptide ligand sequence, refers to less than 100% of the sequence (i.e., 99%, 90%, 80%, 70%, 60% 50% etc.), but comprising 5 or more amino acids or 15 or more nucleotides.

The functional portion of a GPR7 polypeptide of the invention is a receptor which comprises a partial deletion of the complete nucleotide or amino acid sequence and which still maintains the active site(s) and protein domain(s) necessary for the binding of and interaction with a specific ligand comprising a consensus sequence WYKxxAxxxxxT/SVGRAAGLLxGL (referred to herein as a “consensus-containing polypeptide”), preferably L6L, L6C, L7′, L7′L, L7, L7C, L8, L8L, L8C, ppL7, and ppL8 (see FIG. 3).

The functional portion of a GPR8 polypeptide of the invention is a receptor which comprises a partial deletion of the complete nucleotide or amino acid sequence and which still maintains the active site(s) and protein domain(s) necessary for the binding of and interaction with a specific ligand comprising a consensus sequence WYKxxAxxxxxT/SVGRAAGLLxGL, preferably L6L, L6C, L7′, L7′L, L7, L8L, ppL7, and ppL8 (see FIG. 3).

The functional portion of a polypeptide ligand according to the invention can be a polypeptide ligand which comprises a partial deletion of the complete nucleotide or amino acid sequence and which still maintains the active site(s) and protein domain(s) necessary for the binding of and interaction with a specific receptor, preferably GPR7 and/or GPR8.

As used herein, “ligand” refers to a moiety that is capable of associating or binding to a receptor. According to the method of the invention, a ligand and a receptor have a binding constant that is sufficiently strong to allow detection of binding by an assay method that is appropriate for detection of a ligand binding to a receptor (e.g. a second messenger assay to detect an increase or decrease in the production of a second messenger in response to ligand binding to the receptor, a binding assay to measure protein-ligand binding or an immunoassay to measure antibody-antigen interactions). A ligand according to the invention includes the actual molecule that binds a receptor (e.g. L7C is the ligand for GPR7, L7 is the ligand for GPR8) or a ligand may be any nucleotide, antibody, antigen, enzyme, peptide, polypeptide or nucleic acid capable of binding to the receptor. A ligand for GPR7 can comprise a consensus sequence WYKxxAxxxxxT/SVGRAAGLLxGL. Non-limiting examples include polypeptides L6L, L6C, L7′, L7′L, L7, L7C, L8, L8L, L8C, ppL7, and ppL8, a homologue thereof and/or a functional portion thereof. A ligand for GPR8 can comprise a consensus sequence WYKxxAxxxxxT/SVGRAAGLLxGL. Non-limiting examples include polypeptides L6L, L6C, L7′, L7′L, L7, L8L, ppL7, and ppL8, a homologue thereof and/or a functional portion thereof. The ligand may also include a polypeptide, a small chemical substance or a nucleic acid sequence. According to the method of the invention, a ligand and receptor specifically bind to each other (e.g. via covalent or hydrogen bonding or via an interaction between, for example, a protein and a ligand, an antibody and an antigen or protein subunits).

As used herein, the term “detectably labeled” refers to the property of a molecule, that has a structural modification that incorporates a functional group (label) that can be readily detected. Detectable labels include but are not limited to fluorescent compounds, quenchers of fluoresence, isotopic compounds, chemiluminescent compounds, quantum dot labels, biotin, enzymes, electron-dense reagents, and haptens or proteins for which antisera or monoclonal antibodies are available. The various means of detection include but are not limited to spectroscopic, photochemical, radiochemical, biochemical, immunochemical, or chemical means.

As used herein, “GPR7 activity” refers to the activity of a receptor comprising the sequence presented in FIG. 1 (SEQ ID NO: 23), or of a sequence that is homologous to the sequence presented in FIG. 1. “GPR7 activity” also refers to the activity of a “GPR7 polypeptide” as the term is defined herein. More specifically, “GPR7 activity” refers to the binding of one or more ligands comprising a consensus sequence WYKxxAxxxxxT/SVGRAAGLLxGL or a polypeptide selected from among L6L, L6C, L7′, L7′L, L7, L7C, L8, L8L, L8C, ppL7, and ppL8, a homologue thereof and/or a functional portion thereof. “GPR7 activity” also refers to signalling activity exhibited by a GPR7 polypeptide as defined herein.

As used herein, “GPR8 activity” refers to the activity of a receptor comprising the sequence presented in FIG. 3 (SEQ ID NO: 29), or of a sequence that is homologous to the sequence of SEQ ID NO: 29. “GPR8 activity” also refers to the activity of a “GPR8 polypeptide” as the term is defined herein. More specifically, “GPR8 activity” refers to the binding of one or more ligands comprising a consensus sequence WYKxxAxxxxxT/SVGRAAGLLxGL or a polypeptide selected from among L6L, L6C, L7′, L7′L, L7, L8L, ppL7, and ppL8, a homologue thereof and/or a functional portion thereof. “GPR8 activity” also refers to signalling activity exhibited by a GPR8 polypeptide as defined herein.

Homologous sequences of a sequence according to the invention may include an amino acid or nucleotide sequence encoding a similar receptor or polypeptide ligand which exists in other animal species (rat, mouse, cat, dog, etc.) or in specific human population groups, but which are involved in the same biochemical pathway.

Such homologous sequences may comprise additions, deletions or substitutions of one or more amino acids or nucleotides, which do not substantially alter the functional characteristics of the receptor or polypeptide ligand according to the invention. As used herein “does not substantially alter” means that a polypeptide comprising a sequence difference still binds the same ligand(s) or receptor(s) as the original sequence and with the same realtive binding preference(s). Further, the homolog will participate in the same biochemical pathway as the polypeptide to which it is homologous.

A homologous sequence which may exist in other mammal species or specific groups of human populations, where homology indicates sequence identity, means a sequence which presents a high sequence identity (more than 80%, 85%, 90%, 95% or 98% sequence identity) with the complete human nucleotide or amino acid sequence described hereafter, and is preferably characterized by the same pharmacology, especially a preference for binding to L6L, L6C, L7′, L7′L, L7, L7C, L8, L8L, L8C, ppL7, and ppL8 in the case of GPR7.

A homologous sequence which may exist in other mammal species or specific groups of human populations, where homology indicates sequence identity, may also mean a sequence which presents a high sequence identity (more than 80%, 85%, 90%, 95% or 98% sequence identity) with the complete human nucleotide or amino acid sequence described hereafter, and is preferably characterized by the same pharmacology, especially a preference for binding to L6L, L6C, L7′, L7′L, L7, L8L, ppL7, and ppL8 in the case of GPR8.

A homologous sequence according to the present invention means also any sequence which presents a high sequence identity (more than 50%, 65%, 70%, 75%, 80%, 85%, 90%, 95% or 98% sequence identity) with the complete human nucleotide or amino acid sequence described hereafter, and is preferably characterized by the same pharmacology.

Alternatively, an homologous sequence may be any amino acid or nucleotide sequence that exhibits an homology of more than 50%, 55%, 60%, 65%, 70%, 75%, 80%, 85%, 90%, or 95% with the parent sequence, said homology calculated using known rnethods.

Alternatively, an homologous sequence may also be any amino acid sequence resulting from allowed substitutions at any number of positions of the parent sequence according to the formula below:

Ser substituted by one of Ser, Thr, Gly, and Asn;

Arg substituted by one of Arg, His, Gln, Lys, and Glu;

Leu substituted by one of Leu, Ile, Phe, Tyr, Met, and Val;

Pro substituted by one of Pro, Gly, Ala, and Thr;

Thr substituted by one of Thr, Pro, Ser, Ala, Gly, His, and Gln;

Ala substituted by one of Ala, Gly, Thr, and Pro;

Val substituted by one of Val, Met, Tyr, Phe, Ile, and Leu;

Gly substituted by one of Gly, Ala, Thr, Pro, and Ser;

Ile substituted by one of Ile, Met, Tyr, Phe, Val, and Leu;

Phe substituted by one of Phe, Trp, Met, Tyr, Ile, Val, and Leu;

Tyr substituted by one of Tyr, Trp, Met, Phe, Ile, Val, and Leu;

His substituted by one of His, Glu, Lys, Gln, Thr, and Arg;

Gin substituted by one of Gln, Glu, Lys, Asn, His, Thr, and Arg;

Asn substituted by one of Asn, Glu, Asp, Gln, and Ser;

Lys substituted by one of Lys, Glu, Gln, His, and Arg;

Asp substituted by one of Asp, Glu, and Asn;

Glu substituted by one of Glu, Asp, Lys, Asn, Gln, His, and Arg;

Met substituted by one of Met, Phe, Ile, Val, Leu, and Tyr.

In the case of GPR7 and GPR8, homologous sequences can also be nucleotide sequences of more than 400, 600, 800 or 1000 nucleotides able to hybridize to the complete human sequence under stringent hybridisation conditions (such as the ones described by SAMBROOK et al., Molecular Cloning, Laboratory Manuel, Cold Spring, Harbor Laboratory press, New York).

In the case of polypeptide ligands, homologous sequences can also be nucleotide sequences of more than 15, 20, 25, 30, 40, 50, 70, 90, 110, 130, 150, 200, 250, 300, 400, 600, 800 or 1000 nucleotides able to hybridize to the parent sequence under stringent hybridisation conditions (such as the ones described by SAMBROOK et al., Molecular Cloning, Laboratory Manuel, Cold Spring, Harbor Laboratory press, New York).

Another aspect of the present invention is related to a method for the screening, detection and possible recovery of candidate modulators of a receptor of the invention comprising the steps of contacting a cell expressing GPR7 under conditions which permit binding of a polypeptide that comprises a consensus sequence WYKxxAxxxxxT/SVGRAAGLLxGL or one or more of L6L, L6C, L7′, L7′L, L7, L7C, L8, L8L, L8C, ppL7, and ppL8 to GPR7, in the presence of the candidate modulator, performing a second messenger assay, and comparing the results of the second messenger assay obtained in the presence and absence of the candidate modulator.

Another aspect of the present invention is related to a method for the screening, detection and possible recovery of candidate modulators of a receptor of the invention comprising the steps of contacting a cell expressing GPR8 under conditions which permit binding of a polypeptide comprising a consensus sequence WYKxxAxxxxxT/SVGRAAGLLxGL or one or more of L6L, L6C, L7′, L7′L, L7, L8L, ppL7, and ppL8 to GPR8, in the presence of the candidate modulator, performing a second messenger assay, and comparing the results of the second messenger assay obtained in the presence and absence of the candidate modulator.

Another aspect of the present invention is related to a method for the screening, detection and possible recovery of candidate modulators of a receptor of the invention comprising the steps of: contacting a cell membrane expressing GPR7 under conditions which permit binding of a polypeptide comprising a consensus sequence WYKxxAxxxxxT/SVGRAAGLLxGL or one or more of L6L, L6C, L7′, L7′L, L7, L7C, L8, L8L, L8C, ppL7, and ppL8 to GPR7, performing a second messenger assay, and comparing the results of the second messenger assay obtained in the presence and absence of the candidate modulator.

Another aspect of the present invention is related to a method for the screening, detection and possible recovery of candidate modulators of a receptor of the invention comprising the steps of: contacting a cell membrane expressing GPR8 under conditions which permit binding of a polypeptide comprising a consensus sequence WYKxxAxxxxxT/SVGRAAGLLxGL or one or more of L6L, L6C, L7′, L7′L, L7, L8L, ppL7, and ppL8 to GPR8, performing a second messenger assay, and comparing the results of the second messenger assay obtained in the presence and absence of the candidate modulator.

A further aspect of the present invention is related to the unknown agonist and/or antagonist compounds identified and/or recovered by the method of the invention, as well as to a diagnostic kit comprising said (unknown) compounds or a pharmaceutical composition (including a vaccine) comprising an adequate pharmaceutical carrier and a sufficient amount of said (unknown) compound.

An antagonist compound according to the invention means a molecule or a group of molecules able to bind to the receptor according to the invention and block the binding of other ligands (e.g. block the binding of a polyepeptide comprising a consensus sequence WYKxxAxxxxxT/SVGRAAGLLxGL or at least one of L6L, L6C, L7′, L7′L, L7, L7C, L8, L8L, L8C, ppL7, or ppL8).

The invention further encompasses a method of detecting the presence, in a sample, of an agent that modulates the function of GPR7, the method comprising: a) contacting a GPR7 polypeptide with the sample; b) detecting a signalling activity of the GPR7 polypeptide in the presence of the sample; and c) comparing the activity measured in the presence of the sample to the activity measured in a reaction with GPR7 polypeptide and a polypeptide that comprises a consensus sequence WYKxxAxxxxxT/SVGRAAGLLxGL or one or more of L6L, L6C, L7′, L7′L, L7, L7C, L8, L8L, L8C, ppL7, or ppL8 at EC₅₀, wherein an agent that modulates the function of GPR7 is detected if the amount of the GPR7-specific activity measured in the presence of the sample is at least 5%, 10%, 15%, 20% or 25% that of the amount induced by L6L, L6C, L7′, L7′L, L7, L7C, L8, L8L, L8C, ppL7, or ppL8 present at its EC₅₀.

The invention further encompasses a method of detecting the presence, in a sample, of an agent that modulates the function of GPR8, the method comprising: a) contacting a GPR8 polypeptide with the sample; b) detecting a signalling activity of the GPR8 polypeptide in the presence of the sample; and c) comparing the activity measured in the presence of the sample to the activity measured in a reaction with GPR8 polypeptide and a polypeptide comprising a consensus sequence WYKxxAxxxxxT/SVGRAAGLLxGL or one or more of L6L, L6C, L7′, L7′L, L7, L8L, ppL7, or ppL8 at EC₅₀, wherein an agent that modulates the function of GPR8 is detected if the amount of the GPR8-specific activity measured in the presence of the sample is at least 5%, 10%, 15%, 20% or 25% that of the amount induced by L6L, L6C, L7′, L7′L, L7, L8L, ppL7, or ppL8 present at its EC₅₀.

The invention further encompasses a method of diagnosing a disease or disorder characterized by dysregulation of GPR7 signalling, the method comprising: a) contacting a tissue sample with an antibody specific for a GPR7 ligand, preferably L6L, L6C, L7′, L7′L, L7, L7C, L8, L8L, L8C, ppL7, or ppL8, a homology thereof and/or a functional portion thereof; b) detecting binding of the antibody to the tissue sample; and c) comparing the binding detected in step (b) with a standard, wherein a difference in binding relative to the standard is diagnostic of a disease or disorder characterized by dysregulation of GPR7.

The invention further encompasses a method of diagnosing a disease or disorder characterized by dysregulation of GPR8 signalling, the method comprising: a) contacting a tissue sample with an antibody specific for a GPR8 ligand, preferably L6L, L6C, L7′, L7′L, L7, L8L, ppL7, or ppL8, a homology thereof and/or a functional portion thereof; b) detecting binding of the antibody to the tissue sample; and c) comparing the binding detected in step (b) with a standard, wherein a difference in binding relative to the standard is diagnostic of a disease or disorder characterized by dysregulation of GPR8.

The invention further encompasses a method of diagnosing a disease or disorder characterized by dysregulation of GPR7 signalling, the method comprising: a) contacting a tissue sample with an antibody specific for a GPR7 polypeptide and an antibody specific for a GPR7 ligand, preferably L6L, L6C, L7′, L7′L, L7, L7C, L8, L8L, L8C, ppL7, or ppL8, a homology thereof and/or a functional portion thereof; b) detecting binding of the antibodies to the tissue sample; and c) comparing the binding detected in step (b) with a standard, wherein a difference in binding of either antibody or both, relative to the standard, is diagnostic of a disease or disorder characterized by dysregulation of GPR7.

The invention further encompasses a method of diagnosing a disease or disorder characterized by dysregulation of GPR8 signalling, the method comprising: a) contacting a tissue sample with an antibody specific for a GPR8 polypeptide and an antibody specific for a GPR8 ligand, preferably L6L, L6C, L7′, L7′L, L7, L8L, ppL7 or ppL8, a homology thereof and/or a functional portion thereof; b) detecting binding of the antibodies to the tissue sample; and c) comparing the binding detected in step (b) with a standard, wherein a difference in binding of either antibody or both, relative to the standard, is diagnostic of a disease or disorder characterized by dysregulation of GPR8.

The invention further encompasses a method of diagnosing a disease or disorder characterized by dysregulation of GPR7 signalling, the method comprising: a) isolating a tissue sample; b) measuring the concentration of a polypeptide comprising a consensus sequence WYKxxAxxxxxT/SVGRAAGLLxGL or one or more of L6L, ′L6C, L7′, L7′L, L7, L7C, L8, L8L, L8C, ppL7, or ppL8; and c) comparing the amount of the polypeptide comprising a consensus sequence WYKxxAxxxxxT/SVGRAAGLLxGL or one or more of L6L, L6C, L7′, L7′L, L7, L7C, L8, L8L, L8C, ppL7, or ppL8 measured in step (b) with a standard, wherein a difference in the amount of a polypeptide comprising a consensus sequence WYKxxAxxxxxT/SVGRAAGLLxGL or one or more of L6L, L6C, L7′, L7′L, L7, L7C, L8, L8L, L8C, ppL7, or ppL8 relative to the standard is diagnostic of a disease or disorder characterized by dysregulation of GPR7.

The invention further encompasses a method of diagnosing a disease or disorder characterized by dysregulation of GPR8 signalling, the method comprising: a) isolating a tissue sample; b) measuring the concentration of a polypeptide comprising a consensus sequence WYKxxAxxxxxT/SVGRAAGLLxGL or one or more of L6L, L6C, L7′, L7′L, L7, L8L, ppL7, or ppL8; and c) comparing the amount of the polypeptide comprising a consensus sequence WYKxxAxxxxxT/SVGRAAGLLxGL or one or more of L6L, L6C, L7′, L7′L, L7, L8L, ppL7, or ppL8 measured in step (b) with a standard, wherein a difference in the amount of the polypeptide comprising a consensus sequence WYKxxAxxxxxT/SVGRAAGLLxGL or one or more of L6L, L6C, L7′, L7′L, L7, L8L, ppL7, or ppL8 relative to the standard is diagnostic of a disease or disorder characterized by dysregulation of GPR8.

The invention further encompasses a method of diagnosing a disease or disorder characterized by dysregulation of GPR7 signalling, the method comprising: a) isolating nucleic acid from a tissue sample; b) amplifying a GPR7 polynucleotide, using said nucleic acid as a template; and c) comparing the sequence of said amplified GPR7 polynucleotide produced in step (b) with a standard, wherein a difference in said sequence, relative to said standard is diagnostic of a disease or disorder characterized by dysregulation of GPR7.

The invention further encompasses a method of diagnosing a disease or disorder characterized by dysregulation of GPR8 signalling, the method comprising: a) isolating nucleic acid from a tissue sample; b) amplifying a GPR8 polynucleotide, using said nucleic acid as a template; and c) comparing the sequence of said amplified GPR7 polynucleotide produced in step (b) with a standard, wherein a difference in said sequence, relative to said standard is diagnostic of a disease or disorder characterized by dysregulation of GPR8.

In a preferred embodiment, the step of amplifying comprises RT/PCR. In another preferred embodiment, the step of comparing the sequence comprises minisequencing.

A further aspect of the present invention is related to a transgenic non-human mammal, comprising a homologous recombination (knock-out) of the polynucleotide encoding the GPR7 receptor according to the invention or a transgenic non-human mammal over expressing the polypeptide above the natural level of expression. A further aspect of the present invention is also related to a transgenic non-human mammal, comprising a homologous recombination (knock-out) of the polynucleotide encoding the GPR8 receptor according to the invention or a transgenic non-human mammal over expressing the polypeptide above the natural level of expression. As used herein, “above the natural level of expression” refers to a level that is at least 2-fold, preferably 5-fold, more preferably 10-fold and most preferably 100-fold or more (i.e., 150-fold, 200-fold, 250-fold, 500-fold, 1000-fold, 10,000-fold etc.) as compared to the level of expression of the endogenous receptor. A transgenic non-human mammal can be obtained by a method well known by a person skilled in the art, for instance, as described in document WO 98/20112 using the classical technique based upon the transfection of embryonic stem cells, preferably according to the method described by Carmeliet et al. (Nature, Vol.380, p.435-439, 1996).

“Gene targeting” is a type of homologous recombination that occurs when a fragment of genomic DNA is introduced into a mammalian cell and that fragment locates and recombines with endogenous homologous sequences as exemplified in U.S. Pat. No. 5,464,764, and U.S. Pat. No. 5,777,195, the contents of which are hereby incorporated by reference herein in their entireties. As used herein the term “transgenic animal” refers to a non-human animal in which one or more, and preferably essentially all, of the cells of the animal contain a transgene introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Preferably, the transgenic non-human mammal overexpressing the polynucleotide encoding the GPR7 or GPR8 receptor according to the invention comprises the polynucleotide incorporated in a DNA construct with an inducible promoter allowing the overexpression of the receptor and possibly also tissue and cell-specific regulatory elements.

A further aspect of the present invention is related to a transgenic non-human mammal, comprising a homologous recombination (knock-out) of the polynucleotide encoding a polypeptide comprising a consensus sequence WYKxxAxxxxxT/SVGRAAGLLxGL or one of L6L, L6C, L7′, L7′L, L7, L7C, L8, L8L, L8C, ppL7, or ppL8 ligands according to the invention or a transgenic non-human mammal over expressing the polypeptide above the natural level of expression. As used herein, “above the natural level of expression” refers to a level that is at least 2-fold, preferably 5-fold, more preferably 10-fold and most preferably 100-fold or more (i.e., 150-fold, 200-fold, 250-fold, 500-fold, 1000-fold, 10,000-fold etc.) as compared to the level of expression of the endogenous ligands. A transgenic non-human mammal can be obtained by a method well known by a person skilled in the art, for instance, as described in document WO 98/20112 using the classical technique based upon the transfection of embryonic stem cells, preferably according to the method described by Carmeliet et al. (Nature, Vol.380, p.435-439, 1996).

“Gene targeting” is a type of homologous recombination that occurs when a fragment of genomic DNA is introduced into a mammalian cell and that fragment locates and recombines with endogenous homologous sequences as exemplified in U.S. Pat. No. 5,464,764, and U.S. Pat. No. 5,777,195, the contents of which are hereby incorporated by reference herein in their entireties. As used herein the term “transgenic animal” refers to a non-human animal in which one or more, and preferably essentially all, of the cells of the animal contain a transgene introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Preferably, the transgenic non-human mammal overexpressing the polynucleotide encoding the polypeptide comprising a consensus sequence WYKxxAxxxxxT/SVGRAAGLLxGL or one ofL6L, L6C, L7′, L7′L, L7, L7C, L8, L8L, L8C, ppL7, or ppL8 ligands according to the invention comprises the polynucleotide incorporated in a DNA construct with an inducible promoter allowing the overexpression of the receptor and possibly also tissue and cell-specific regulatory elements.

The diagnostic kit according to one aspect of the invention includes at least GPR7 receptor and, packaged separately, a polypeptide comprising a consensus sequence WYKxxAxxxxxT/SVGRAAGLLxGL or one or more of L6L, L6C, L7′, L7′L, L7, L7C, L8, L8L, L8C, ppL7, or ppL8 and also may comprise advantageously all the necessary means and media for performing a detection of specific binding (for example of L7C) to the GPR7 receptor of the invention and possibly correlating the detection of specific binding to a method of monitoring of one or more of the symptoms of the diseases described hereafter.

The diagnostic kit according to another aspect of the invention includes at least GPR8 receptor and, packaged separately, a polypeptide comprising a consensus sequence WYKxxAxxxxxT/SVGRAAGLLxGL or one or more of L6L, L6C, L7′, L7′L, L7, L8L, ppL7, or ppL8 and also may comprise advantageously all the necessary means and media for performing a detection of specific binding (for example of L7) to the GPR8 receptor of the invention and possibly correlating the detection of specific binding to a method of monitoring of one or more of the symptoms of the diseases described hereafter.

Possibly, the kit comprises elements for a specific diagnostic or dosage of such bound compounds through high throughput screening techniques, well known to the person skilled in the art, especially the one described in WO 00/02045. The high throughput screening diagnostic dosage and monitoring can be performed by using various solid supports, such as microtiter plates or biochips selected by the person skilled in the art.

In the pharmaceutical composition according to the invention, the adequate pharmaceutical carrier is a carrier of solid, liquid or gaseous form, which can be selected by the person skilled in the art according to the type of administration and the possible side effects of the compound according to the invention. The ratio between the pharmaceutical carrier and the specific compound can be selected by the person skilled in the art according to the patient treated, the administration and the possible side effects of the compound, as well as the type of disease of disorder treated or submitted to a specific prevention.

Pharmaceutical compositions as described herein can be used to treat and/or prevent diseases or disorders related to GPR7 and GPR8 dysfunction in tissues shown in Table 1. That is, an increase or decrease in GPR7 or GPR8 or their respective signalling activities in a tissue or tissues can correlate with one or more disease phenotypes affecting those tissues.

Based upon the strong expression of both receptors in the CNS, the pharmaceutical composition the pharmaceutical compositions can be used to treat and/or prevent various diseases or disorders, preferably selected from the group consisting of ostatic hypertrophy, migraine, vomiting, psychotic and neurological disorders, including anxiety, schizophrenia, manic depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome. Expression in the immune system indicates that pharmaceutical compositions can be used for the treatment and/or prevention of diseases including autoimmune and inflammatory diseases as well as thrombosis and other cardiovascular diseases.

Among the mentioned diseases the preferred applications are related to therapeutic agents targeting 7TM receptors that can play a function in preventing, improving or correcting dysfunctions or diseases including, but not limited to those related to fertility, fetal development, infections such as bacterial, fungal, protozoan and viral infections, particularly infections caused by HIV1 and HIV2, pain, cancer, anorexia, bulimia, asthma, Parkinson's disease, acute heart failure, hypertension, urinary retention, osteoporosis, angina pectoris, myocardial infarction, ulcers, asthma, allergies, benign prostatic hypertrophy, psychotic and neurological disorders including anxiety, depression, migraine, vomiting, stroke, schizophrenia, manic depression, delirium, dementia, severe mental retardation and dyskinesias, such as Huntington's disease or Gilles de la Tourette's syndrome including thrombosis and other cardiovascular diseases, autoimmune and inflammatory diseases.

As used herein, an “antagonist” is a ligand which competitively binds to the receptor at the same site as an agonist, but does not activate an intracellular response initiated by an active form of a receptor, and thereby inhibits the intracellular response induced by an agonist, for example L7C or L7, by at least 10%, preferably 15-25%, more preferably 25-50% and most preferably, 50-100%, as compared to the intracellular response in the presence of an agonist and in the absence of an antagonist.

As used herein, an “agonist” refers to a ligand, that activates an intracellular response when for example, it binds to a receptor at concentrations equal or lower to L7C concentrations which induce an intracellular response. An agonist according to the invention may increase the intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc.), as compared to the intracellular response in the absence of agonist. An agonist, according to the invention may decrease internalization of a cell surface receptor such that the cell surface expression of a receptor is increased by at least 2-fold, preferably 5-fold, more preferably 10-fold and most preferably, 100-fold or more (i.e., 150-fold, 200-fold, 250-fold, 500-fold, 1000-fold, 10,000-fold etc.), as compared to the number of cell surface receptors present on the surface of a cell in the absence of an agonist. In another embodiment of the invention, an agonist stablizes a cell surface receptor and increases the cell surface expression of a receptor by at least 2-fold, preferably 5-fold, more preferably 10-fold and most preferably, 100-fold or more (i.e., 200-fold, 250-fold, 500-fold, 1000-fold, 10,000-fold etc.), as compared to the number of cell surface receptors present on the surface of a cell in the absence of agonist.

As used herein, an “inverse agonist” refers to a ligand which decreases a constitutive activity of a cell surface receptor when it binds to a receptor. An inverse agonist according to the invention may decrease the constitutive intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc.), as compared to the intracellular response in the absence of inverse agonist.

An “inhibitor” compound according to the invention is a molecule directed against the receptor or against the natural ligand for the receptor that decreases the binding of the ligand to the receptor by at least 10%, preferably 15-25%, more preferably 25-50% and most preferably, 50-100%, in the presence of L6L, L6C, L7′, L7′L, L7, L7C, L8, L8L, L8C, ppL7, or ppL8, as compared to the binding in the presence of L6L, L6C, L7′, L7′L, L7, L7C, L8, L8L, L8C, ppL7, or ppL8 and in the absence of inhibitor. “inhibitor” compound of the invention can decrease the intracellular response induced by an agonist, for example L6L, L6C, L7′, L7′L, L7, L7C, L8, L8L, L8C, ppL7, or ppL8, by at least 10%, preferably 15-25%, more preferably 25-50% and most preferably, 50-100%. An “inhibitor” also refers to a nucleotide sequence encoding an inhibitor compound of the invention.

As used herein, “natural ligand” refers to a naturally occurring ligand, found in nature, which binds to a receptor in a manner that is equivalent to L6L, L6C, L7′, L7′L, L7, L7C, L8, L8L, L8C, ppL7, or ppL8. A “natural ligand” does not refer to an engineered ligand that is not found in nature and that is engineered to bind to a receptor, where it did not formerly do so in a manner different, either in degree or kind, from that which it was engineered to do, it is no longer naturally-occurring but is “non-natural” and is derived from a naturally occurring molecule.

As used herein, a “modulator” refers to any compound that increases or decreases the cell surface expression of a receptor of the invention, increases or decreases the binding of a ligand to a receptor of the invention, or any compound that increases or decreases the intracellular response initiated by an active form of the receptor of the invention, either in the presence or absence of an agonist, and in the presence of a ligand for the receptor, for example L6L, L6C, L7′, L7′L, L7, L7C, L8, L8L, L8C, ppL7, or ppL8. A modulator includes an agonist, antagonist, inhibitor or inverse agonist, as defined herein. A modulator can be a protein, a nucleic acid, an antibody or fragment thereof, a peptide, etc. Candidate modulators can be natural or synthetic compounds, including, for example, small molecules, compounds contained in extracts of animal, plant, bacterial or fungal cells, as well as conditioned medium from such cells.

As used herein, the term “small molecule” refers to a compound having molecular mass of less than 3000 Daltons, preferably less than 2000 or 1500, still more preferably less than 1000, and most preferably less than 600 Daltons. A “small organic molecule” is a small molecule that comprises carbon.

As used herein, the term “change in binding” or “change in activity” and the equivalent terms “difference in binding” or “difference in activity” or difference in the amount of “amplified” PCR product refer to an increase or decrease of at least 10% in binding relative to the standard, or in signalling activity or in mRNA levels relative to the standard in a given assay.

As used herein, the term “dysregulation” refers to the signalling activity of, for example, GPR7 in a sample wherein:

a) a 10% increase or decrease in the amount of GPR7 or corresponding polypeptide ligand mRNA or polypeptide levels is measured relative to the standard, as defined herein, of a given assay or;

b) at least a single base pair change in the GPR7 or corresponding polypeptide ligand coding sequence is detected relative to the standard, as defined herein, of a given assay and results in an alteration of GPR signalling activity as defined in paragraphs a), c) or d) or;

c) a 10% increase or decrease in the amount of polypeptide ligand binding activity is measured relative to the standard, as defined herein, of a given assay or;

d) a 10% increase or decrease in secondary messenger assays, as defined herein, is measured relative to the standard, as defined herein, of a given assay.

As used herein, the term “dysregulation” may equally well refer to the signalling activity of, for example, GPR8 in a sample wherein steps a), b), c) and d) above are performed using GPR8 and associated components in place of GPR7.

As used herein, the term “conditions permitting the binding” in reference to one or more ligands and GPR7 or refers to conditions of, for example, temperature, salt concentration, pH and protein concentration under which said ligand binds GPR7. Exact binding conditions will vary depending upon the nature of the assay, for example, whether the assay uses viable cells or only membrane fraction of cells. However, because GPR7 are cell surface proteins, favored conditions will generally include physiological salt (90 mM) and pH (about 7.0 to 8.0). Temperatures for binding can vary from 15° C. to 37° C., but will preferably be between room temperature and about 30° C. The concentration of consensus sequence-comprising polypeptide or L6L, L6C, L7′, L7′L, L7, L7C, L8, L8L, L8C, ppL7, or ppL8 in a binding reaction will also vary, but will preferably be about 1 nM (e.g., in a reaction with radiolabelled tracer L6L, L6C, L7′, L7′L, L7, L7C, L8, L8L, L8C, ppL7, or ppL8 where the concentration is generally below the K_{d}) to 10 mM (e.g., L6L, L6C, L7, L7′L, L7, L7C, L8, L8L, L8C, ppL7, or ppL8 as competitor).

As used herein, the term “conditions permitting the binding” may equally well refer to one or more ligands and GPR8, wherein the above is performed using GPR8 and associated components in place of GPR7.

As used herein, the term “sample” refers to the source of molecules being tested for the presence of an agent or modulator compound that modulates binding to or signalling activity of, for example, a GPR7 or GPR8 polypeptide. A sample can be an environmental sample, a natural extract of animal, plant yeast or bacterial cells or tissues, a clinical sample, a synthetic sample, or a conditioned medium from recombinant cells or a fermentation process. The term “tissue sample” refers to a tissue that is tested for the presence, abundance, quality or an activity of a GPR7 or GPR8 polypeptide, a nucleic acid encoding a GPR7 or GPR8 polypeptide, or an agent or compound that modifies the ligand binding or activity of a GPR7 or GPR8 polypeptide.

As used herein, the term “sample” may equally well refer to the source of molecules being tested for the presence of an agent or modulator compound that modulates binding to or signalling activity of a GPR8 polypeptide wherein the above is performed using GPR8 and associated components in place of GPR7.

As used herein, a “tissue” is an aggregate of cells that perform a particular function in an organism. The term “tissue” as used herein refers to cellular material from a particular physiological region. The cells in a particular tissue can comprise several different cell types. A non-limiting example of this would be brain tissue that further comprises neurons and glial cells, as well as capillary endothelial cells and blood cells, all contained in a given tissue section or sample. In addition to solid tissues, the term “tissue” is also intended to encompass non-solid tissues, such as blood.

As used herein, the term “membrane fraction” refers to a preparation of cellular lipid membranes comprising a GPR7. It may also refer to a preparation of cellular lipid membranes comprising a GPR8. As the term is used herein, a “membrane fraction” is distinct from a cellular homogenate, in that at least a portion (i.e., at least 10%, and preferably more) of non-membrane-associated cellular constituents has been removed. The term “membrane associated” refers to those cellular constituents that are either integrated into a lipid membrane or are physically associated with a component that is integrated into a lipid membrane.

As used herein, the “second messenger assay” preferably comprises the measurement of guanine nucleotide binding or exchange, adenylate cyclase, intra-cellular cAMP, intracellular inositol phosphate, intra-cellular diacylglycerol concentration, arachinoid acid concentration, MAP kinase(s) or tyrosine kinase(s), protein kinase C activity, or reporter gene expression or an aequorin-based assay according to methods known in the art and defined herein.

As used herein, the term “signaling activity” refers to the activation of second messengers. The term “second messenger” refers to a molecule, generated or caused to vary in concentration by the activation of a G-Protein Coupled Receptor, that participates in the transduction of a signal from that GPCR. Non-limiting examples of second messengers include cAMP, diacylglycerol, inositol triphosphate, arachidonic acid release, inositol triphosphates and intracellular calcium. The term “change in the level of a second messenger” refers to an increase or decrease of at least 10%, and preferably more, e.g., 15%, 25%, 50%, 75% or more, including 100% (doubling) or more, e.g., 5-fold, 10-fold, 100-fold or more in the detected level of a given second messenger relative to the amount detected in an assay performed in the absence of a candidate modulator.

As used herein, the term “aequorin-based assay” refers to an assay for GPCR activity that measures intracellular calcium flux induced by activated GPCRs, wherein intracellular calcium flux is measured by the luminescence of aequorin expressed in the cell.

As used herein, the term “binding” refers to the physical association of a ligand (e.g., L7C, L7 or an antibody) with a receptor (e.g., GPR7, GPR8). As the term is used herein, binding is “specific” if it occurs with an EC₅₀ or a K_{d} of 1 μM less, generally in the range of 1 μM to 10 pM. For example, binding is specific if the EC₅₀ or K_{d} is 1 μM or less, 500 or less nM, 100 or less nM, 10 or less nM, 9.5 or less nM, 9 or less nM, 8.5 or less nM, 8 or less nM, 7.5 or less nM, 7 or less nM, 6.5 or less nM, 6 or less nM, 5.5 or less nM, 5 or less nM, 4.5 or less nM, 4 or less nM, 3.5 or less nM, 3 or less nM, 2.5 or less nM, 2 or less nM, 1.5 or less nM, 1 or less nM, 750 or less pM, 500 or less pM, 250 or less pM or 100 or less pM or less.

As used herein, the term “EC₅₀” in reference to GPR7 refers to that concentration of a compound at which a given activity, including binding of L7C or other ligand and a functional activity of a GPR7 polypeptide, is 50% of the maximum for that GPR7 activity measurable using the same assay in the absence of compound. The above cited activity may also include the binding of L7 or other ligand and a functional activity of a GPR8 polypeptide, in which case the “EC₅₀” would refer to that concentration of compound at which a given activity is 50% of the maximum for that GPR8 activity measurable using the same assay in the absence of compound. Stated differently, the “EC₅₀” is the concentration of compound that gives 50% activation, when 100% activation is set at the amount of activity that does not increase with the addition of more agonist. It should be noted that the EC₅₀ of consensus-containing polypeptide or L6L, L6C, L7′, L7′L, L7, L7C, L8, L8L, L8C, ppL7, or ppL8 will vary according to the identity of the consensus-containing polypeptide or L6L, L6C, L7′, L7′L, L7, L7C, L8, L8L, L8C, ppL7, or ppL8 analogue used in the assay; for example, L6L, L6C, L7′, L7′L, L7, L7C, L8, L8L, L8C, ppL7, or ppL8 analogues can have EC₅₀ values higher than, lower than or the same as L6L, L6C, L7′, L7′L, L7, L7C, L8, L8L, L8C, ppL7, or ppL8. Therefore, where a L6L, L6C, L7′, L7′L, L7, L7C, L8, L8L, L8C, ppL7, or ppL8 analogue differs from L6L, L6C, L7′, L7′L, L7, L7C, L8, L8L, L8C, ppL7, or ppL8, one of the skill in the art can determine the EC₅₀ for that analogue according to conventional methods. The EC₅₀ of a given consensus-containing polypeptide or L6L, L6C, L7′, L7′L, L7, L7C, L8, L8L, L8C, ppL7, or ppL8 analog is measured by performing an assay for the activity of a fixed amount of GPR7 or GPR8 polypeptide in the presence of doses of the consensus-containing polypeptide or L6L, L6C, L7′, L7′L, L7, L7C, L8, L8L, L8C, ppL7, or ppL8 that increase at least until the GPR7 or GPR8 response is saturated or maximal, and then plotting the measured GPR7 or GPR8 activity versus the concentration of consensus-containing polypeptide or L6L, L6C, L7′, L7′L, L7, L7C, L8, L8L, L8C, ppL7, or ppL8.

As used herein, the term “saturation” refers to the concentration of L6L, L6C, L7′, L7′L, L7, L7C, L8, L8L, L8C, ppL7, or ppL8 or other ligand at which further increases in ligand concentration fail to increase the binding of L6, L6L, L7′, L7′L, L7, L7AL, L8, L8L, L8C, ppL7, or ppL8 ligand or GPR7- or GPR8-specific signalling activity.

As used herein, the term “IC₅₀”, may refer to the GPR7 receptor and is the concentration of an antagonist or inverse agonist that reduces the maximal activation of a GPR7 receptor by 50%.

As used herein, the term “IC₅₀”, may also refer to the GPR8 receptor and is the concentration of an antagonist or inverse agonist that reduces the maximal activation of a GPR8 receptor by 50%.

As used herein, the term “decrease in binding” refers to a decrease of at least 10% in the amount of binding detected in a given assay with a known or suspected modulator of GPR7 or GPR8 relative to binding detected in an assay lacking that known or suspected modulator.

As used herein, the term “delivering,” when used in reference to a drug or agent, means the addition of the drug or agent to an assay mixture, or to a cell in culture. The term also refers to the administration of the drug or agent to an animal. Such administration can be, for example, by injection (in a suitable carrier, e.g., sterile saline or water) or by inhalation, or by an oral, transdermal, rectal, vaginal, or other common route of drug administration.

As used herein, the term “standard” refers to a sample taken from an individual who is not affected by a disease or disorder characterized by dysregulation of GPR7 activity. The “standard” is used as a reference for the comparison of GPR7 mRNA levels and quality (i.e., mutant vs. wild type), as well as for the comparison of GPR7 activities.

As used herein, the term “standard” may also refer to a sample taken from an individual who is not affected by a disease or disorder characterized by dysregulation of GPR8 activity. The “standard” is used therein as a reference for the comparison of GPR8 mRNA levels and quality (i.e., mutant vs. wild type), as well as for the comparison of GPR8 activities.

As used herein, the term “amplifying,” when applied to a nucleic acid sequence, refers to a process whereby one or more copies of a nucleic acid sequence is generated from a template nucleic acid. A preferred method of “amplifying” is PCR or RT/PCR.

As used herein, the term “G-Protein coupled receptor,” or “GPCR” refers to a membrane-associated polypeptide with 7 alpha helical transmembrane domains. Functional GPCR's associate with a ligand or agonist and also associate with and activate G-proteins. GPR7 and GPR8 are both GPCRs.

As used herein, the term “antibody” is the conventional immunoglobulin molecule, as well as fragments thereof which are also specifically reactive with one of the subject polypeptides. Antibodies can be fragmented using conventional techniques and the fragments screened for utility in the same manner as described herein below for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanised molecules having affinity for a polypeptide conferred by at least one CDR region of the antibody. In preferred embodiments, the antibody further comprises a label attached thereto and is able to be detected, (e.g., the label can be a radioisotope, fluorescent compound, chemiluminescent compound, enzyme, or enzyme co-factor). The antibodies, monoclonal or polyclonal and its hypervariable portion thereof (FAB, FAB″, etc.) as well as the hybridoma cell producing the antibodies are a further aspect of the present invention which find a specific industrial application in the field of diagnostics and monitoring of specific diseases, preferably the ones hereafter described.

Inhibitors according to the invention include but are not limited to labeled monoclonal or polyclonal antibodies or hypervariable portions of the antibodies.

As used herein, the term “transgenic animal” refers to any animal, preferably a non-human mammal (e.g., a rodent (mouse, rat, etc.), porcine, bovine, ovine, etc.), bird, fish or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

Sequences

The invention relates to the nucleotide and amino acid sequences encoding GPR7 (presented in FIG. 1). The invention also relates to sequences that are homologous to the nucleotide and amino acid sequences encoding GPR7.

It further relates to the nucleotide and amino acid sequences encoding GPR8 (presented in FIG. 3). The invention also relates to sequences that are homologous to the nucleotide and amino acid sequences encoding GPR8.

i) Calculation of Sequence Homology

Sequence identity with respect to any of the sequences presented herein can be determined by a simple “eyeball” comparison (i.e. a strict comparison) of any one or more of the sequences with another sequence to see if that other sequence has, for example, at least 70% sequence identity to the sequence(s).

Relative sequence identity can also be determined by commercially available computer programs that can calculate percentage identity between two or more sequences using any suitable algorithm for determining identity, using for example default parameters. A typical example of such a computer program is CLUSTAL. Other computer program methods to determine identity and similarity between two sequences include but are not limited to the GCG program package (Devereux et al 1984 Nucleic Acids Research 12: 387) and FASTA (Altschul et al 1990 J Molec Biol 403-410).

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (Ausubel et al., 1995, Short Protocols in Molecular Biology, 3rd Edition, John Wiley & Sons), FASTA (Atschul et al., 1990, J. Mol. Biol., 403-410) and the GENEWORKS suite of comparison tools. Both BLAST and FASTA are available for offline and online searching (Ausubel et al., 1999 supra, pages 7-58 to 7-60).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see http://www.ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available at http://www.ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided at http://www.ncbi.nlm.nih.gov/BLAST. In some embodiments of the present invention, no gap penalties are used when determining sequence identity.

ii) Hybridization

The present invention also encompasses nucleotide sequences that are capable of hybridizing to the sequences presented herein, or any fragment or derivative thereof, or to the complement of any of the above.

Hybridization means a “process by which a strand of nucleic acid joins with a complementary strand through base pairing” (Coombs J (1994) Dictionary of Biotechnology, Stockton Press, New York N.Y.) as well as the process of amplification as carried out in polymerase chain reaction technologies as described in Dieffenbach C W and G S Dveksler (1995, PCR Primer, a Laboratory Manual, Cold Spring Harbor Press, Plainview N.Y.).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Nucleotide sequences of the invention capable of selectively hybridizing to the nucleotide sequences presented herein, or to their complement, will be generally at least 70%, preferably at least 75%, more preferably at least 85 or 90% and even more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” or “specifically hybridizing” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, and preferably less than 100 fold as intense as the specific interaction observed with the target DNA. The intensity of interaction may be measured, for example, by radiolabelling the probe, e.g. with ³²P.

Also included within the scope of the present invention are nucleotide sequences that are capable of hybridizing to the nucleotide sequences presented herein under conditions of intermediate to maximal stringency. Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, maximum stringency hybridization can be used to identify or detect identical nucleotide sequences while an intermediate (or low) stringency hybridization can be used to identify or detect similar or related nucleotide sequences.

In a preferred embodiment, the present invention covers nucleotide sequences that can hybridize to one or more of the Tramell GPCR nucleotide sequences of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

The present invention also encompasses nucleotide sequences that are capable of hybridizing to the sequences that are complementary to the sequences presented herein, or any fragment or derivative thereof Likewise, the present invention encompasses nucleotide sequences that are complementary to sequences that are capable of hybridizing to the sequence of the present invention. These types of nucleotide sequences are examples of variant nucleotide sequences. In this respect, the term “variant” encompasses sequences that are complementary to sequences that are capable of hydridizing to the nucleotide sequences presented herein. Preferably, however, the term “variant” encompasses sequences that are complementary to sequences that are capable of hydridizing under stringent conditions (e.g., 65° C. and 0.1×SSC {1×SSC=0.15 M NaCl, 0.015 Na₃ citrate pH 7.0}) to the nucleotide sequences presented herein.

Cells

A cell that is useful according to the invention is preferably selected from the group consisting of bacterial cells, yeast cells, insect cells or mammal cells.

A cell that is useful according to the invention can be any cell into which a nucleic acid sequence encoding a receptor according to the invention can be introduced such that the receptor is expressed at natural levels or above natural levels, as defined herein. Preferably a receptor of the invention that is expressed in a cell exhibits normal or near normal pharmacology, as defined herein. Most preferably a receptor of the invention that is expressed in a cell comprises the nucleotide or amino acid sequence presented in FIG. 1 or a nucleotide or amino acid sequence that is at least 70% identical to the amino acid sequence presented in FIG. 1.

According to a preferred embodiment of the present invention, a cell is selected from the group consisting of COS7-cells, a CHO cell, a LM (TK−) cell, a NIH-3T3 cell, HEK-293 cell, K-562 cell or a 1321N1 astrocytoma cell but also other transfectable cell lines.

Assays for the Identification of Agents that Modulate the Activity of GPR7 or GPR8

Agents that modulate the activity of GPR7 and/or GPR8 can be identified in a number of ways that take advantage of the interaction of the receptor with a polypeptide comprising a consensus sequence WYKxxAxxxxxT/SVGRAAGLLxGLL6L, or with one of L6C, L7′, L7′L, L7, L7C, L8, L8L, L8C, ppL7, or ppL8. For example, the ability to reconstitute GPR7/L7C binding either in vitro, on cultured cells or in vivo provides a target for the identification of agents that disrupt that binding. In a further example, the ability to reconstitute GPR8/L7 binding either in vitro, on cultured cells or in vivo provides a target for the identification of agents that disrupt that binding. Assays based on disruption of binding can identify agents, such as small organic molecules, from libraries or collections of such molecules. Alternatively, such assays can identify agents in samples or extracts from natural sources, e.g., plant, fungal or bacterial extracts or even in human tissue samples (e.g., tumour tissue). In one aspect, the extracts can be made from cells expressing a library of variant nucleic acids, peptides or polypeptides. Modulators of GPR7/L7C and/or GPR8/L7 binding can then be screened using a binding assay or a functional assay that measures downstream signalling through the receptor.

Another approach that uses the GPR7/L7C interaction more directly to identify agents that modulate GPR7 function measures changes in GPR7 downstream signalling induced by candidate agents or candidate modulators. The same approach may be used to identify agents that modulate GPR8, by using the GPR8/L7 interaction. These functional assays can be performed in isolated cell membrane fractions or on cells expressing the receptor on their surfaces. The finding that a consensus sequence WYKxxAxxxxxT/SVGRAAGLLxGL and specific polypeptides L6L, L6C, L7′, L7′L, L7, L7C, L8, L8L, L8C, ppL7, or ppL8 are ligands of the GPR7 receptor and/or of the GPR8 receptor permits screening assays to identify agonists, antagonists and inverse agonists of receptor activity. For each of the approaches or assays described below using L6L, L6C, L7′, L7′L, L7, L7C, L8, L8L, L8C, ppL7, or ppL8, one can alternatively employ a polypeptide comprising a consensus sequence WYKxxAxxxxxT/SVGRAAGLLxGL. The screening assays will have two general approaches.

i) Ligand binding assays, in which cells expressing GPR7, membrane extracts from such cells, or immobilized lipid membranes comprising GPR7 are exposed to labelled L6L, L6C, L7′, L7′L, L7, L7C, L8, L8L, L8C, ppL7, or ppL8 and candidate compound. Following incubation, the reaction mixture is measured for specific binding of the labelled to the GPR7 receptor. Compounds that interfere with binding or displace labelled L6L, L6C, L7′, L7′L, L7, L7C, L8, L8L, L8C, ppL7, or ppL8 can be agonists, antagonists or inverse agonists of GPR7 activity. Subsequent functional analysis can then be performed on positive compounds to determine in which of these categories they belong.

In another example of ligand binding assays, cells expressing GPR8, membrane extracts from such cells, or immobilized lipid membranes comprising GPR8 are exposed to labelled L6L, L6C, L7′, L7′L, L7, L8C, ppL7, or ppL8 and candidate compound. Following incubation, the reaction mixture is measured for specific binding of the labelled to the GPR8 receptor. Compounds that interfere with binding or displace labelled L6L, L6C, L7′, L7′L, L7, L8L, ppL7, or ppL8 can be agonists, antagonists or inverse agonists of GPR8 activity. Subsequent functional analysis can then be performed on positive compounds to determine in which of these categories they belong.

ii) Functional Assays, in which a Signalling Activity of GPR7 or GPR8 is Measured.

a) For agonist screening, cells expressing GPR7 or membranes prepared from them are incubated with a candidate compound, and a signalling activity of GPR7 is measured. The activity induced by compounds that modulate receptor activity is compared to that induced by L6L, L6C, L7′, L7′L, L7, L7C, L8, L8L, L8C, ppL7, or ppL8. An agonist or partial agonist will have a maximal biological activity corresponding to at least 10% of the maximal activity of L7C when the agonist or partial agonist is present at 1 mM or less, and preferably will have a potency which is at least as potent than L6L, L6C, L7′, L7′L, L7, L7C, L8, L8L, L8C, ppL7, or ppL8.

In another example of agonist screening, cells expressing GPR8 or membranes prepared from them are incubated with a candidate compound, and a signalling activity of GPR8 is measured. The activity induced by compounds that modulate receptor activity is compared to that induced by L6L, L6C, L7′, L7′L, L7, L8L, ppL7, or ppL8. An agonist or partial agonist will have a maximal biological activity corresponding to at least 10% of the maximal activity of L7 when the agonist or partial agonist is present at 1 mM or less, and preferably will have a potency which is at least as potent than L6L, L6C, L7′, L7′L, L7, L8L, ppL7, or ppL8.

b) For antagonist or inverse agonist screening, cells expressing GPR7 or membranes isolated from them are assayed for signalling activity in the presence of L6L, L6C, L7′, L7′L, L7, L7C, L8, L8L, L8C, ppL7, or ppL8 with or without a candidate compound. Antagonists will reduce the level of L7C-stimulated receptor activity by at least 10%, relative to reactions lacking the antagonist in the presence of L7C. Inverse agonists will reduce the constitutive activity of the receptor by at least 10%, relative to reactions lacking the inverse agonist.

In another example of antagonist or inverse agonist screening, cells expressing GPR8 or membranes isolated from them are assayed for signalling activity in the presence of L6L, L6C, L7′, L7′L, L7, L8L, ppL7, or ppL8 with or without a candidate compound. Antagonists will reduce the level of L7-stimulated receptor activity by at least 10%, relative to reactions lacking the antagonist in the presence of L7. Inverse agonists will reduce the constitutive activity of the receptor by at least 10%, relative to reactions lacking the inverse agonist.

c) For inverse agonist screening, cells expressing constitutive GPR7 activity or membranes isolated from them are used in a functional assay that measures an activity of the receptor in the presence of a candidate compound. Inverse agonists are those compounds that reduce the constitutive activity of the receptor by at least 10%. Overexpression of GPR7 may lead to constitutive activation. GPR7 can be overexpressed by placing it under the control of a strong constitutive promoter, e.g., the CMV early promoter. Alternatively, certain mutations of conserved GPCR amino acids or amino acid domains tend to lead to constitutive activity. See for example: Kjelsberg et al., 1992, J. Biol. Chem. 267:1430; McWhinney et al., 2000. J. Biol. Chem. 275:2087; Ren et al., 1993, J. Biol. Chem. 268:16483; Samama et al., 1993, J.Biol.Chem 268:4625; Parma et al., 1993, Nature 365:649; Parma et al., 1998, J. Pharmacol. Exp.Ther. 286:85; and Parent et al., 1996, J. Biol. Chem. 271:7949.

In another example of inverse agonist screening, cells expressing constitutive GPR8 activity or membranes isolated from them are used in a functional assay that measures an activity of the receptor in the presence of a candidate compound. Inverse agonists are those compounds that reduce the constitutive activity of the receptor by at least 10%. Overexpression of GPR8 may lead to constitutive activation. GPR8 can be overexpressed by placing it under the control of a strong constitutive promoter, e.g., the CMV early promoter.

iii) Ligand Binding and Displacement Assays:

One can use GPR7 polypeptides expressed on a cell, or isolated membranes containing receptor polypeptides, along with L6L, L6C, L7′, L7′L, L7, L7C, L8, L8L, L8C, ppL7, or ppL8 in order to screen for compounds that inhibit the binding of L6L, L6C, L7′, L7′L, L7, L7C, L8, L8L, L8C, ppL7, or ppL8 to GPR7. When identified in an assay that measures binding or L6L, L6C, L7′, L7′L, L7, L7C, L8, L8L, L8C, ppL7, or ppL8 displacement alone, compounds will have to be subjected to functional testing to determine whether they act as agonists, antagonists or inverse agonists.

One can may also use GPR8 polypeptides expressed on a cell, or isolated membranes containing receptor polypeptides, along with L6L, L6C, L7′, L7′L, L7, L8L, ppL7, or ppL8 in order to screen for compounds that inhibit the binding of L6L, L6C, L7′, L7′L, L7, L8L, ppL7, or ppL8 to GPR8. When identified in an assay that measures binding or L6L, L6C, L7′, L7′L, L7, L8L, ppL7, or ppL8 displacement alone, compounds will have to be subjected to functional testing to determine whether they act as agonists, antagonists or inverse agonists.

For displacement experiments, cells expressing a GPR7 polypeptide (generally 25,000 cells per assay or 1 to 100 μg of membrane extracts) are incubated in binding buffer with, for example, labelled L7C in the presence or absence of increasing concentrations of a candidate modulator. To validate and calibrate the assay, control competition reactions using increasing concentrations of unlabeled L7C can be performed. After incubation, cells are washed extensively, and bound, labelled L7C is measured as appropriate for the given label (e.g., scintillation counting, fluorescence, etc.). A decrease of at least 10% in the amount of labelled L7C bound in the presence of candidate modulator indicates displacement of binding by the candidate modulator. Candidate modulators are considered to bind specifically in this or other assays described herein if they displace 50% of labelled L7C (sub-saturating L7C dose) at a concentration of 1 μM or less.

Alternatively, for displacement experiments, cells expressing a GPR8 polypeptide (generally 25,000 cells per assay or 1 to 100 μg of membrane extracts) are incubated in binding buffer with, for example, labelled L7 in the presence or absence of increasing concentrations of a candidate modulator. To validate and calibrate the assay, control competition reactions using increasing concentrations of unlabeled L7 can be performed. After incubation, cells are washed extensively, and bound, labelled L7 is measured as appropriate for the given label (e.g., scintillation counting, fluorescence, etc.). A decrease of at least 10% in the amount of labelled L7 bound in the presence of candidate modulator indicates displacement of binding by the candidate modulator. Candidate modulators are considered to bind specifically in this or other assays described herein if they displace 50% of labelled L7 (sub-saturating L7 dose) at a concentration of 1 μM or less.

Alternatively, binding or displacement of binding can be monitored by surface plasmon resonance (SPR). Surface plasmon resonance assays can be used as a quantitative method to measure binding between two molecules by the change in mass near an immobilized sensor caused by the binding or loss of binding of, for example, L7C from the aqueous phase to a GPR7 polypeptide immobilized in a membrane on the sensor, or for example, L7 from the aqueous phase to a GPR8 polypeptide immobilized in a membrane on the sensor. This change in mass is measured as resonance units versus time after injection or removal of the L6L, L6C, L7′, L7′L, L7, L7C, L8, L8L, L8C, ppL7, or ppL8 or candidate modulator and is measured using a Biacore Biosensor (Biacore AB). GPR7 or GPR8 can be immobilized on a sensor chip (for example, research grade CM5 chip; Biacore AB) in a thin film lipid membrane according to methods described by Salamon et al. (Salamon et al., 1996, Biophys J. 71: 283-294; Salamon et al., 2001, Biophys. J. 80: 1557-1567; Salamon et al., 1999, Trends Biochem. Sci. 24: 213-219, each of which is incorporated herein by reference.). Sarrio et al. demonstrated that SPR can be used to detect ligand binding to the GPCR A(1) adenosine receptor immobilized in a lipid layer on the chip (Sarrio et al., 2000, Mol. Cell. Biol. 20: 5164-5174, incorporated herein by reference). Conditions for L7C binding to GPR7 and/or GPR8 in an SPR assay can be fine-tuned by one of skill in the art using the conditions reported by Sarrio et al. as a starting point.

SPR can assay for modulators of binding in at least two ways. First, L7C, for example, can be pre-bound to immobilized polypeptide—GPR7 for example—followed by injection of candidate modulator at a concentration ranging from 0.1 nM to 1 μM. Displacement of the bound L7C can be quantitated, permitting detection of modulator binding. Alternatively, the membrane-bound GPR7 polypeptide can be pre-incubated with a candidate modulator and challenged with for example L7C. A difference in binding affinity between L7C and GPR7 pre-incubated with the modulator, compared with that between L7C and GPR7 in absence of the modulator will demonstrate binding or displacement of L7C in the presence of modulator. In either assay, a decrease of 10% or more in the amount of L7C bound is in the presence of candidate modulator, relative to the amount of a L7C bound in the absence of candidate modulator indicates that the candidate modulator inhibits the interaction of GPR7 and L7C. The SPR assay above may be performed with any combination of receptor and ligand described herein such as GPR8 and L7.

Another method of detecting inhibition of binding of, for example, L7C to GPR7 uses fluorescence resonance energy transfer (FRET). FRET is a quantum mechanical phenomenon that occurs between a fluorescence donor (D) and a fluorescence acceptor (A) in close proximity to each other (usually<100 Å of separation) if the emission spectrum of D overlaps with the excitation spectrum of A. The molecules to be tested, e.g. L7C and a GPR7 polypeptide, are labelled with a complementary pair of donor and acceptor fluorophores. While bound closely together by the GPR7: L7C interaction, the fluorescence emitted upon excitation of the donor fluorophore will have a different wavelength from that emitted in response to that excitation wavelength when the L7C and GPR7 polypeptide are not bound, providing for quantitation of bound versus unbound molecules by measurement of emission intensity at each wavelength. Donor fluorophores with which to label the GPR7 polypeptide are well known in the art. Of particular interest are variants of the A. Victoria GFP known as Cyan FP (CFP, Donor (D)) and Yellow FP (YFP, Acceptor (A)). As an example, the YFP variant can be made as a fusion protein with GPR7. Vectors for the expression of GFP variants as fusions (Clontech) as well as flurophore-labeled L7C compounds (Molecular Probes) are known in the art. The addition of a candidate modulator to the mixture of fluorescently-labelled L7C and YFP-GPR7 protein will result in an inhibition of energy transfer evidenced by, for example, a decrease in YFP fluorescence relative to a sample without the candidate modulator. In an assay using FRET for the detection of GPR7: L7C interaction, a 10% or greater decrease in the intensity of fluorescent emission at the acceptor wavelength in samples containing a candidate modulator, relative to samples without the candidate modulator, indicates that the candidate modulator inhibits the GPR7: L7C interaction. The FRET assay above may be performed with any combination of receptor and ligand described herein such as GPR8 and L7.

A variation on FRET uses fluorescence quenching to monitor molecular interactions. One molecule in the interacting pair can be labelled with a fluorophore, and the other with a molecule that quenches the fluorescence of the fluorophore when brought into close apposition with it. A change in fluorescence upon excitation is indicative of a change in the association of the molecules tagged with the fluorophore:quencher pair. Generally, an increase in fluorescence of the labelled GPR7 polypeptide is indicative that the L7C molecule bearing the quencher has been displaced. For quenching assays, a 10% or greater increase in the intensity of fluorescent emission in samples containing a candidate modulator, relative to samples without the candidate modulator, indicates that the candidate modulator inhibits GPR7: L7C interaction. The fluorescence quenching assay above may be performed with any combination of receptor and ligand described herein such as GPR8 and L7.

In addition to the surface plasmon resonance and FRET methods, fluorescence polarization measurement is useful to quantitate binding. The fluorescence polarization value for a fluorescently-tagged molecule depends on the rotational correlation time or tumbling rate. Complexes, such as those formed by GPR7 associating with a fluorescently labelled L7C, have higher polarization values than uncomplexed, labelled L7C. The inclusion of a candidate inhibitor of the GPR7: L7C interaction results in a decrease in fluorescence polarization, relative to a mixture without the candidate inhibitor, if the candidate inhibitor disrupts or inhibits the interaction of GPR7 with L7C. Fluorescence polarization is well suited for the identification of small molecules that disrupt the formation of receptor:ligand complexes. A decrease of 10% or more in fluorescence polarization in samples containing a candidate modulator, relative to fluorescence polarization in a sample lacking the candidate modulator, indicates that the candidate modulator inhibits GPR7: L7C interaction. The fluorescence polarisation assay above may be performed with any combination of receptor and ligand described herein such as GPR8 and L7.

Another alternative for monitoring GPR7: L7C interactions uses a biosensor assay. ICS biosensors have been described in the art (Australian Membrane Biotechnology Research Institute; http//www.ambri.com.au/; Cornell B, Braach-Maksvytis V, King L, Osman P, Raguse B, Wieczorek L, and Pace R. “A biosensor that uses ion-channel switches” Nature 1997, 387, 580). In this technology, the association of GPR7 and its ligand, is coupled to the closing of gramacidin-facilitated ion channels in suspended membrane bilayers and thus to a measurable change in the admittance (similar to impedence) of the biosensor. This approach is linear over six orders of magnitude of admittance change and is ideally suited for large scale, high throughput screening of small molecule combinatorial libraries. A 10% or greater change (increase or decrease) in admittance in a sample containing a candidate modulator, relative to the admittance of a sample lacking the candidate modulator, indicates that the candidate modulator inhibits the interaction of GPR7 and L7C. It is important to note that in assays testing the interaction of GPR7 with L7C, it is possible that a modulator of the interaction need not necessarily interact directly with the domain(s) of the proteins that physically interact with L7C. It is also possible that a modulator will interact at a location removed from the site of interaction and cause, for example, a conformational change in the GPR7 polypeptide. Modulators (inhibitors or agonists) that act in this manner are nonetheless of interest as agents to modulate the activity of GPR7. The biosensor assay above may be performed with any combination of receptor and ligand described herein such as GPR8 and L7.

It should be understood that any of the binding assays described herein can be performed with both related ligands of GPR7 and/or GPR8 (for example, L6L, L6C, L7′, L7′L, L7, L7C, L8, L8L, L8C, ppL7, or ppL8 or others agonist or antagonist, etc.) and non-related ligands of GPR7 and/or GPR8 (e.g., a small molecule identified as described herein or analogues thereof including but not limited to any of the analogues, a natural or synthetic peptide, a polypeptide, an antibody or antigen-binding fragment thereof, a lipid, a carbohydrate, and a small organic molecule).

Any of the binding assays described can be used to determine the presence of an agent in a sample, (e.g., a tissue sample) that binds to the GPR7 receptor molecule, or that affects the binding of, for example, L7C to the receptor. It can also be used to determine the presence of an agent in a sample, that binds to the GPR8 receptor molecule, or that affects the binding of, for example, L7 to the receptor. To do so, GPR7 polypeptide is reacted with L7C or another ligand in the presence or absence of the sample, and L7C or ligand binding is measured as appropriate for the binding assay being used. Alternatively GPR8 polypeptide is reacted with L7 or another ligand in the presence or absence of the sample, and L7 or ligand binding is measured as appropriate for the binding assay being used. A decrease of 10% or more in the binding of L7C (or L7) or other ligand indicates that the sample contains an agent that modulates L7C (or L7) or ligand binding to the receptor polypeptide.

Functional Assays of Receptor Activity

i) GTPase/GTP Binding Assays:

For GPCRs such as GPR7 or GPR8, a measure of receptor activity is the binding of GTP by cell membranes containing receptors. In the method described by Traynor and Nahorski, 1995, Mol. Pharmacol. 47: 848-854, incorporated herein by reference, one essentially measures G-protein coupling to membranes by detecting the binding of labelled GTP. For GTP binding assays, membranes isolated from cells expressing the receptor are incubated in a buffer containing 20 mM HEPES, pH 7.4, 100 mM NaCl, and mM MgCl2, 100 pM ³⁵S-GTPγS and 10 μM GDP. The assay mixture is incubated for 60 minutes at 30° C., after which unbound labelled GTP is removed by filtration onto GF/B filters. Bound, labelled GTP is measured by liquid scintillation counting. In order to assay for modulation of, for example, L7C-induced GPR7 activity, membranes prepared from cells expressing a GPR7 polypeptide are mixed with L7C, and the GTP binding assay is performed in the presence and absence of a candidate modulator of GPR7 activity. An increase of 10% or more in labelled GTP binding as measured by scintillation counting in an assay of this kind containing a candidate modulator, relative to an assay without the modulator, indicates that the candidate modulator inhibits GPR7 activity. A similar GTP-binding assay can be performed without L7C to identify compounds that act as agonists. In this case, L7C-stimulated GTP binding is used as a standard. A compound is considered as an agonist if it induces at least 50% of the level of GTP binding induced by L7C when the compound is present at 10 μM or less, and preferably will induce a level the same as or higher than that induced by L7C.

GTPase activity may be measured by incubating the membranes containing a for example, GPR7 polypeptide with γ³²P-GTP. Active GTPase will release the label as inorganic phosphate, which is detected by separation of free inorganic phosphate in a 5% suspension of activated charcoal in 20 mM H₃PO₄, followed by scintillation counting. Controls include assays using membranes isolated from cells not expressing GPR7 (mock-transfected), in order to exclude possible non-specific effects of the candidate compound.

In order to assay for the effect of a candidate modulator on, for example, GPR7-regulated GTPase activity, membrane samples are incubated with L7C, with and without the modulator, followed by the GTPase assay. A change (increase or decrease) of 10% or more in the level GTPase activity relative to samples without modulator is indicative of GPR7 modulation by a candidate modulator.

The GTPase/GTP assay above may be performed with any combination of receptor and ligand described herein such as GPR8 and L7.

ii. Downstream Pathway Activation Assays:

a. Calcium Flux—the Aequorin-Based Assay.

The aequorin assay takes advantage of the responsiveness of mitochondrial apoaequorin to intracellular calcium release induced by the activation of GPCRs (Stables et al., 1997, Anal. Biochem. 252:115-126; Detheux et al., 2000, J. Exp. Med., 192 1501-1508; both of which are incorporated herein by reference). Briefly, clones which express GPR7, for example, are transfected to coexpress mitochondrial apoaequorin and Gα16. Cells are incubated with 5 μM Coelenterazine H (Molecular Probes) for 4 hours at room temperature, washed in DMEM-F12 culture medium and resuspended at a concentration of 0.5×10⁶ cells/ml. Cells are then mixed with test agonist molecules and light emission by the aequorin is recorded with a luminometer for 30 sec. Results are expressed as Relative Light Units (RLU). Controls include assays using membranes isolated from cells not expressing GPR7 (mock transfected), in order to exclude possible non-specific effects of the candidate compound.

Aequorin activity or intracellular calcium levels are considered “changed” if light intensity increases or decreases by 10% or more in a sample of cells, expressing a GPR7 polypeptide and treated with a candidate modulator, relative to a sample of cells expressing the GPR7 polypeptide but not treated with the candidate modulator or relative to a sample of cells not expressing the GPR.7 polypeptide (mock-transfected cells) but treated with the candidate modulator.

When performed in the absence of, for example, L7C, the assay can be used to identify an agonist of GPR7 activity. When the assay is performed in the presence of, for example, L7C, it can be used to assay for an antagonist.

The GTPase/GTP assay above may be performed with any combination of receptor and ligand described herein such as GPR8 and L7.

b. Adenylate Cyclase Assay:

Assays for adenylate cyclase activity are described by Kenimer & Nirenberg, 1981, Mol. Pharmacol. 20: 585-591, incorporated herein by reference. That assay is a modification of the assay taught by Solomon et al., 1974, Anal. Biochem. 58: 541-548, also incorporated herein by reference. Briefly, 100 μl reactions contain 50 mM Tris-HCl (pH 7.5), 5 mM MgCl₂, 20 mM creatine phosphate (disodium salt), 10 units (71 μg of protein) of creatine phosphokinase, 1 mM α-³²P-ATP (tetrasodium salt, 2 μCi), 0.5 mM cyclic AMP, G-³H-labeled cyclic AMP (approximately 10,000 cpm), 0.5 mM Ro20-1724, 0.25% ethanol, and 50-200 μg of protein homogenate to be tested (e.g., homogenate from cells expressing or not expressing a GPR7 polypeptide, treated or not treated with L7C with or without a candidate modulator). Reaction mixtures are generally incubated at 37° C. for 60 minutes. Following incubation, reaction mixtures are deproteinized by the addition of 0.9 ml of cold 6% trichloroacetic acid. Tubes are centrifuged at 1800×g for 20 minutes and each supernatant solution is added to a Dowex AG50W-X4 column. The cAMP fraction from the column is eluted with 4 ml of 0.1 mM imidazole-HCl (pH 7.5) into a counting vial. Assays should be performed in triplicate. Control reactions should also be performed using protein homogenate from cells that do not express a GPR7 polypeptide.

According to the invention, adenylate cyclase activity is considered “changed” if it increases or decreases by 10% or more in a sample taken from cells treated with a candidate modulator of GPR7 activity, relative to a similar sample of cells not treated with the candidate modulator or relative to a sample of cells not expressing the GPR7 polypeptide (mock-transfected cells) but treated with the candidate modulator.

The adenylate cyclase assay above may be performed with any combination of receptor and ligand described herein such as GPR8 and L7.

c. cAMP Assay:

Intracellular or extracellular cAMP is measured using a cAMP radioimmunoassay (RIA) or cAMP binding protein according to methods widely known in the art. For example, Horton & Baxendale, 1995, Methods Mol. Biol. 41: 91-105, which is incorporated herein by reference, describes an RIA for cAMP.

A number of kits for the measurement of cAMP are commercially available, such as the High Efficiency Fluorescence Polarization-based homogeneous assay marketed by LJL Biosystems and NEN Life Science Products. Control reactions should be performed using extracts of mock-transfected cells to exclude possible non-specific effects of some candidate modulators.

The level of cAMP is considered “changed” if the level of cAMP detected in cells expressing a receptor polypeptide such as, for example, GPR7, said cells treated with a candidate modulator of GPR7 activity (or in extracts of such cells), using the RIA-based assay of Horton & Baxendale, 1995, supra, increases or decreases by at least 10% relative to the cAMP level in similar cells not treated with the candidate modulator.

The cAMP assay above may be performed with any receptor described herein such as GPR8.

d. Phospholipid Breakdown, DAG Production and Inositol Triphosphate Levels:

Receptors that activate the breakdown of phospholipids can be monitored for changes due to the activity of known or suspected modulators of GPR7 and/or GPR8 by monitoring phospholipid breakdown, and the resulting production of second messengers DAG and/or inositol triphosphate (IP₃). Methods of detecting each of these are described in Phospholipid Signalling Protocols edited by Ian M. Bird. Totowa, N.J., Humana Press, 1998, which is incorporated herein by reference. See also Rudolph et al., 1999, J. Biol. Chem. 274: 11824-11831, incorporated herein by reference, which also describes an assay for phosphatidylinositol breakdown. Assays might be performed using cells or extracts of cells expressing a receptor polypeptide, such as, for example GPR7, said cells treated or not treated with a ligand peptide such as for example L7C, with or without a candidate modulator. Control reactions should be performed using mock-transfected cells, or extracts from them in order to exclude possible non-specific effects of some candidate modulators.

According to the invention, phosphatidylinositol breakdown, and diacylglycerol and/or inositol triphosphate levels are considered “changed” if they increase or decrease by at least 10% in a sample from cells expressing a GPR7 polypeptide and treated with a candidate modulator, relative to the level observed in a sample from cells expressing a GPR7 polypeptide that is not treated with the candidate modulator.

The assay involving phospholipid breakdown, DAG production and inositol triphosphate levels as described above may be performed with any combination of receptor and ligand described herein such as GPR8 and L7.

e. PKC Activation Assays:

Growth factor receptor tyrosine kinases can signal via a pathway involving activation of Protein Kinase C (PKC), which is a family of phospholipid- and calcium-activated protein kinases. PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC can be used to monitor PKC activation and thereby receptor activity. In addition, the activity of receptors that signal via PKC can be monitored through the use of reporter gene constructs driven by the control sequences of genes activated by PKC activation. This type of reporter gene-based assay is discussed in more detail below.

For a more direct measure of PKC activity, the method of Kikkawa et al., 1982, J. Biol. Chem. 257: 13341, incorporated herein by reference, can be used. This assay measures phosphorylation of a PKC substrate peptide, which is subsequently separated by binding to phosphocellulose paper. This PKC assay system can be used to measure activity of purified kinase, or the activity in crude cellular extracts. Protein kinase C sample can be diluted in 20 mM HEPES/ 2 mM DTT immediately prior to assay.

The substrate for the assay is the peptide Ac-FKKSFKL-NH2, derived from the myristoylated alanine-rich protein kinase C substrate protein (MARCKS). The Kₘ of the enzyme for this peptide is approximately 50 μM. Other basic, protein kinase C-selective peptides known in the art can also be used, at a concentration of at least 2-3 times their Kₘ. Cofactors required for the assay include calcium, magnesium, ATP, phosphatidylserine and diacylglycerol. Depending upon the intent of the user, the assay can be performed to determine the amount of non-activated PKC present (activating conditions) or the amount of active PKC present (non-activating conditions). For most purposes according to the invention, non-activating conditions will be used, such that the PKC, that is active in the sample when it is isolated, is measured, rather than measuring the PKC that can be activated. For non-activating conditions, calcium is omitted from the assay in favor of EGTA.

The assay is performed in a mixture containing 20 mM HEPES, pH 7.4, 1-2 mM DTT, 5 mM MgCl₂, 100 μM ATP, ˜1 μCi γ-³²P-ATP, 100 μg/ml peptide substrate (˜100 μM), 140 μM/3.8 μM phosphatidylserine/diacylglycerol membranes, and 100 μM calcium (or 500 μM EGTA). 48 μl of sample, diluted in 20 mM HEPES, pH 7.4, 2 mM DTT is used in a final reaction volume of 80 μl. Reactions are performed at 30° C. for 5-minutes, followed by addition of 25 μl of 100 mM ATP, 100 mM EDTA, pH 8.0, which stops the reactions.

After the reaction is stopped, a portion (85 μl) of each reaction is spotted onto a Whatman P81 cellulose phosphate filter, followed by washes: four times 500 ml in 0.4% phosphoric acid, (5-10 min per wash); and a final wash in 500 ml 95% EtOH, for 2-5 min. Bound radioactivity is measured by scintillation counting. Specific activity (cpm/nmol) of the labelled ATP is determined by spotting a sample of the reaction onto P81 paper and counting without washing. Units of PKC activity, defined as nmol phosphate transferred per min, are calculated as follows:

The activity, in UNITS (nmol/min) is:

$$= \frac{( cpm on paper ) \times ( 105 µl total / 85 µl spotted )}{( assay time , min ) ( specific activity of ATP cpm / nmol ) . }$$

An alternative assay can be performed using a Protein Kinase C Assay Kit sold by Pan Vera (Cat. #P2747).

Assays are performed on extracts from cells expressing a receptor polypeptide such as, for example, a GPR7 polypeptide, said cells treated or not treated with a ligand peptide such as, for example, L7C with or without a candidate modulator. Control reactions should be performed using mock-transfected cells, or extracts from them in order to exclude possible non-specific effects of some candidate modulators.

According to the invention, PKC activity is considered “changed” by a candidate modulator when the units of PKC measured by either assay described above increase- or decrease by at least 10%, in extracts from cells expressing GPR7 and treated with a candidate modulator, relative to a reaction performed on a similar sample from cells not treated with a candidate modulator.

The PKC activation assay as described above may be performed with any combination of receptor and ligand described herein such as GPR8 and L7.

f. Kinase Assays:

MAP kinase activity can be assayed using any of several kits available commercially, for example, the p38 MAP Kinase assay kit sold by New England Biolabs (Cat #9820) or the FlashPlate™ MAP Kinase assays sold by Perkin-Elmer Life Sciences.

MAP Kinase activity is considered “changed” if the level of activity is increased or decreased by 10% or more in a sample from cells, expressing a GPR7 polypeptide, treated with a candidate modulator relative to MAP kinase activity in a sample from similar cells not treated with the candidate modulator.

Direct assays for tyrosine kinase activity using known synthetic or natural tyrosine kinase substrates and labelled phosphate are well known, as are similar assays for other types of kinases (e.g., Ser/Thr kinases). Kinase assays can be performed with both purified kinases and crude extracts prepared from cells expressing a GPR7 polypeptide, treated, for example, with or without L7C, with or without a candidate modulator. Control reactions should be performed using mock-transfected cells, or extracts from them in order to exclude possible non-specific effects of some candidate modulators. Substrates can be either full-length protein or synthetic peptides representing the substrate. Pinna & Ruzzene (1996, Biochem. Biophys. Acta 1314: 191-225, incorporated herein by reference) list a number of phosphorylation substrate sites useful for detecting kinase activities. A number of kinase substrate peptides are commercially available. One that is particularly useful is the “Src-related peptide,” RRLIEDAEYAARG (available from Sigma #A7433), which is a substrate for many receptor and nonreceptor tyrosine kinases. Because the assay described below requires binding of peptide substrates to filters, the peptide substrates should have a net positive charge to facilitate binding. Generally, peptide substrates should have at least 2 basic residues and a free amino terminus. Reactions generally use a peptide concentration of 0.7-1.5 mM.

Assays are generally carried out in a 25 μl volume comprising 5 μl of 5× kinase buffer (5 mg/mL BSA, 150 mM Tris-Cl (pH 7.5), 100 mM MgCl₂; depending upon the exact kinase assayed for, MnCl₂ can be used in place of or in addition to the MgCl₂), 5 μl of 1.0 mM ATP (0.2 mM final concentration), γ-32P-ATP (100-500 cpm/pmol), 3 μl of mM peptide substrate (1.2 mM final concentration), cell extract containing kinase to be tested (cell extracts used for kinase assays should contain a phosphatase inhibitor (e.g. 0.1-1 mM sodium orthovanadate)), and H₂O to 25 μl. Reactions are performed at 30° C., and are initiated by the addition of the cell extract.

Kinase reactions are performed for 30 seconds to about 30 minutes, followed by the addition of 45 μl of ice-cold 10% trichloroacetic acid (TCA). Samples are spun for 2 minutes in a microcentrifuge, and 35 μl of the supernatant is spotted onto Whatman P81 cellulose phosphate filter circles. The filters are washed three times with 500 ml cold 0.5% phosphoric acid, followed by one wash with 200 ml of acetone at room temperature for 5 minutes. Filters are dried and incorporated ³²P is measured by scintillation counting. The specific activity of ATP in the kinase reaction (e.g., in cpm/pmol) is determined by spotting a small sample (2-5 μl) of the reaction onto a P81 filter circle and counting directly, without washing. Counts per minute obtained in the kinase reaction (minus blank) are then divided by the specific activity to determine the moles of phosphate transferred in the reaction.

Tyrosine kinase activity is considered “changed” if the level of kinase activity is increased or decreased by 10% or more in a sample from cells, expressing a receptor polypeptide, for example GPR7 polypeptide, said cells treated with a candidate modulator relative to kinase activity in a sample from similar cells not treated with the candidate modulator.

The kinase assay as described above may be performed with any receptor described herein such as, for example, GPR8.

g. Transcriptional Reporters for Downstream Pathway Activation:

The intracellular signal initiated by binding of an agonist to a receptor, e.g., GPR7 and/or GPR8, sets in motion a cascade of intracellular events, the ultimate consequence of which is a rapid and detectable change in the transcription or translation of one or more genes. The activity of the receptor can therefore be monitored by detecting the expression of a reporter gene driven by control sequences responsive to GPR7 and/or GPR8 activation.

As used herein “promoter” refers to the transcriptional control elements necessary for receptor-mediated regulation of gene expression, including not only the basal promoter, but also any enhancers or transcription-factor binding sites necessary for receptor-regulated expression. By selecting promoters that are responsive to the intracellular signals resulting from agonist binding, and operatively linking the selected promoters to reporter genes whose transcription, translation or ultimate activity is readily detectable and measurable, the transcription based reporter assay provides a rapid indication of whether a given receptor is activated.

Reporter genes such as luciferase, CAT, GFP, β-lactamase or β-galactosidase are well known in the art, as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful for making reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

An example of a gene that is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation. This characteristic makes the c-fos regulatory regions particularly attractive for use as a reporter of receptor activation.

The c-fos regulatory elements include (see, Verma et al., 1987, Cell 51: 513-514): a TATA box that is required for transcription initiation; two upstream elements for basal transcription, and an enhancer, which includes an element with dyad symmetry and which is required for induction by TPA, serum, EGF, and PMA.

The 20 bp c-fos transcriptional enhancer element located between −317 and −298 bp upstream from the c-fos mRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels can be monitored by detecting either the binding of the transcription factor, or the expression of a reporter gene linked to a CREB-binding element (termed the CRE, or cAMP response element). The DNA sequence of the CRE is TGACGTCA. Reporter constructs responsive to CREB binding activity are described in U.S. Pat. No. 5,919,649.

Other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666); the somatostatin gene promoter (cAMP responsive; Montminy et al., 1986, Proc. Natl. Acad. Sci. 8.3:6682-6686); the proenkephalin promoter (responsive to cAMP, nicotinic agonists, and phorbol esters; Comb et al., 1986, Nature 323:353-356); the phosphoenolpyruvate carboxy-kinase (PEPCK) gene promoter (cAMP responsive; Short et al., 1986, J. Biol. Chem. 261:9721-9726).

Additional examples of transcriptional control elements that are responsive to changes in GPCR activity include, but are not limited to those responsive to the AP-1 transcription factor and those responsive to NF-κB activity. The consensus AP-1 binding site is the palindrome TGA(C/G)TCA (Lee et al., 1987, Nature 325: 368-372; Lee et al., 1987, Cell 49: 741-752). The AP-1 site is also responsible for mediating induction by tumor promoters such as the phorbol ester 12-O-tetradecanoylphorbol-β-acetate (TPA), and are therefore sometimes also referred to as a TRE, for TPA-response element. AP-1 activates numerous genes that are involved in the early response of cells to growth stimuli. Examples of AP-1-responsive genes include, but are not limited to the genes for Fos and Jun (which proteins themselves make up AP-1 activity), Fos-related antigens (Fra) 1 and 2, IκBα, ornithine decarboxylase, and annexins I and II.

The NF-κB binding element has the consensus sequence GGGGACTTTCC. A large number of genes have been identified as NF-κB responsive, and their control elements can be linked to a reporter gene to monitor GPCR activity. A small sample of the genes responsive to NF-κB includes those encoding IL-1β (Hiscott et al., 1993, Mol. Cell. Biol. 13: 6231-6240), TNF-α (Shakhov et al., 1990, J. Exp. Med. 171: 35-47), CCR5 (Liu et al., 1998, AIDS Res. Hum. Retroviruses 14: 1509-1519), P-selection (Pan & McEver, 1995, J. Biol. Chem. 270: 23077-23083), Fas ligand (Matsui et al., 1998, J. Immunol. 161: 3469-3473), GM-CSF (Schreck & Baeuerle, 1990, Mol. Cell. Biol. 10: 1281-1286) and IκBα (Haskill et al., 1991, Cell 65: 1281-1289). Each of these references is incorporated herein by reference. Vectors encoding NF-κB-responsive reporters are also known in the art or can be readily made by one of skill in the art using, for example, synthetic NF-κB elements and a minimal promoter, or using the NF-κB-responsive sequences of a gene known to be subject to NF-κB regulation. Further, NF-κB responsive reporter constructs are commercially available from, for example, CLONTECH.

A given promoter construct may be tested by exposing cells expressing a receptor polypeptide such as, for example, GPR7, said cells transfected with the construct, to, for example, L7C. An increase of at least two-fold in the expression of reporter in response to L7C indicates that the reporter is an indicator of GPR7 activity.

Cells that stably express a GPR7 polypeptide are stably transfected with a reporter gene under the control of an inducible promoter. Secondary messengers whose concentration is modified following GPR7 activation by potential agonist, will modulate this promoter. To screen for agonists, the cells are left untreated, exposed to candidate modulators, or exposed, for example, to L7C, and expression of the reporter is measured. The L7C-treated cultures serve as a standard for the level of transcription induced by a known agonist. An increase of at least 50% in reporter expression in the presence of a candidate modulator indicates that the candidate is a modulator of GPR7 activity. An agonist will induce at least as much, and preferably the same amount or more, reporter expression than L7C alone. This approach can also be used to screen for inverse agonists where cells express a GPR7 polypeptide at levels such that there is an elevated basal activity of the reporter in the absence of L7C or another agonist. A decrease in reporter activity of 10% or more in the presence of a candidate modulator, relative to its absence, indicates that the compound is an inverse agonist.

To screen for antagonists, the cells expressing a receptor polypeptide, such as, for example, GPR7, said cells carrying the reporter construct are exposed to a ligand such as, for example L7C (or another agonist) in the presence and absence of candidate modulator. A decrease of 10% or more in reporter expression in the presence of candidate modulator, relative to the absence of the candidate modulator, indicates that the candidate is a modulator of GPR7 activity.

Controls for transcription assays include cells not expressing GPR7 but carrying the reporter construct, as well as cells with a promoterless reporter construct.

Compounds that are identified as modulators of GPR7-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate GPR7 activity. The libraries can be, for example, cDNA libraries from natural sources, e.g., plants, animals, bacteria, etc., or they can be libraries expressing randomly or systematically mutated variants of one or more polypeptides. Genomic libraries in viral vectors can also be used to express the mRNA content of one cell or tissue, in the different libraries used for screening of GPR7.

Assay which rely on transcriptional reporters for downstream pathway activation as described above may be performed with any combination of receptor and ligand described herein such as GPR8 and L7.

h) Inositol Phosphates (IP) Measurement

Cells of the invention, for example, CHO—K1 cells, are labelled for 24 hours with μCi/ml [³H] inositol in inositol free DMEM containing 5% FCS, antibiotics, amphotericin, sodium pyruvate and 400 μg/ml G418. Cells are incubated for 2 h in Krebs-Ringer Hepes (KRH) buffer of the following composition (124 mM NaCl, 5 mM KCl, 1.25 mM MgSO₄, 1.45 mM CaCl₂, 1.25 mM KH₂PO₄, 25 mM Hepes (pH:7.4) and 8 mM glucose). The cells are then challenged with various SCFA for 30 min. The incubation is stopped by the addition of an ice cold 3% perchloric acid solution. IP are extracted and separated on Dowex columns as previously described (25).

GPR7 and GPR8 Assays

The invention provides for an assay for detecting the activity of a receptor of the invention in a sample. For example, GPR7 activity can be measured in a sample comprising a cell or a cell membrane that expresses GPR7. The assay is performed by incubating the sample in the presence or absence of L7C (or another agonist) and carrying out a second messenger assay, as described above. The results of the second messenger assay performed in the presence of L7C (or another agonist) are compared with those performed in the absence of L7C (or another agonist) to determine whether the receptor (GPR7) is active. An increase of 10% or more in the detected level of a given second messenger, as defined herein, in the presence of L7C (or another agonist) relative to the amount detected in an assay performed in the absence of L7C (or another agonist) is indicative of GPR7 activity.

In another example, GPR8 activity can be measured in a sample comprising a cell or a cell membrane that expresses GPR8. The assay is performed by incubating the sample in the presence or absence of L7 (or another agonist) and carrying out a second messenger assay, as described above. The results of the second messenger assay performed in the presence of L7 (or another agonist) are compared with those performed in the absence of L7 (or another agonist) to determine whether the receptor (GPR8) is active. An increase of 10% or more in the detected level of a given second messenger, as defined herein, in the presence of L7 (or another agonist) relative to the amount detected in an assay performed in the absence of L7 (or another agonist) is indicative of GPR8 activity.

Any of the assays of receptor activity, including but not limited to the GTP-binding, GTPase, adenylate cyclase, cAMP, phospholipid-breakdown, diacylglycerol, inositol triphosphate, arachidonic acid release (see below), PKC, kinase and transcriptional reporter assays, can be used to determine the presence of an agent in a sample, e.g., a tissue sample, that affects the activity of the GPR7 and/or GPR8 receptor molecule. To do so, GPR7 polypeptide, for example, is assayed for activity in the presence and absence of the sample or an extract of the sample. An increase in receptor activity (e.g. GPR7) in the presence of the sample or extract relative to the absence of the sample indicates that the sample contains an agonist of the receptor activity. A decrease in receptor activity in the presence of L7C or another agonist and the sample, relative to receptor activity in the presence of L7C alone, indicates that the sample contains an antagonist of the receptor (e.g. GPR7). If desired, samples can then be fractionated and further tested to isolate or purify the agonist or antagonist. The amount of increase or decrease in measured activity necessary for a sample to be said to contain a modulator depends upon the type of assay used. Generally, a 10% or greater change (increase or decrease) relative to an assay performed in the absence of a sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation than with L7C alone.

Alternatively, GPR8 polypeptide, for example, is assayed for activity in the presence and absence of the sample or an extract of the sample. An increase in receptor activity (e.g. GPR8) in the presence of the sample or extract relative to the absence of the sample indicates that the sample contains an agonist of the receptor activity. A decrease in receptor activity in the presence of L7 or another agonist and the sample, relative to receptor activity in the presence of L7 alone, indicates that the sample contains an antagonist of the receptor (e.g. GPR8). If desired, samples can then be fractionated and further tested to isolate or purify the agonist or antagonist. The amount of increase or decrease in measured activity necessary for a sample to be said to contain a modulator depends upon the type of assay used. Generally, a 10% or greater change (increase or decrease) relative to an assay performed in the absence of a sample indicates the presence of a modulator in the sample. One exception is the transcriptional reporter assay, in which at least a two-fold increase or 10% decrease in signal is necessary for a sample to be said to contain a modulator. It is preferred that an agonist stimulates at least 50%, and preferably 75% or 100% or more, e.g., 2-fold, 5-fold, 10-fold or greater receptor activation than with L7C alone.

Other functional assays include, for example, microphysiometer or biosensor assays (see Haftier, 2000, Biosens. Bioelectron. 15: 149-158, incorporated herein by reference). The intracellular level of arachinoid acid can also be determined as described in Gijon et al., 2000, J. Biol. Chem., 275:20146-20156.

Diagnostic Assays Based upon the Interactions of GPR7 and/or GPR8 with an Agonist (e.g. L7C or L7):

Signalling through GPCRs is instrumental in the pathology of a large number of diseases and disorders. GPR7, which is expressed in cells of the cerebellum, frontal cortex, hypothalamus, pituitary gland, amygdala, brain, spinal cord can have a role in all cerebral disorders or diseases. GPR7 is also expressed in liver, testis, colon, trachea, rectum and small intestine and therefore can have a role in all disorders or diseases related to these organs. GPR8, which is expressed at high levels in cells of the caudate nucleus, hippocampus, amygdala, and at moderate levels in the adult brain, thalamus, parietal cortex, pituitary gland, adrenal gland, lymph nodes and lymphoblastic leukaemia, and therefore may be related to diseases involving all these cells, tissues and/or organs.

The expression pattern of GPR7 and GPR8 and the knowledge with respect to disorders generally mediated by GPCRs suggests that GPR7 and GPR8 can be involved in disturbances of cell migration, cancer, development of tumours and tumour metastasis, inflammatory and neo-plastic processes, wound and bone healing and dysfunction of regulatory growth functions, diabetes, obesity, anorexia, bulimia, acute heart failure, hypotension, hypertension, urinary retention, osteoporosis, angina pectoris, myocardial infarction, restenosis, atherosclerosis, thrombosis and other cardiovascular diseases, autoimmune and inflammatory diseases, diseases characterized by excessive smooth muscle cell proliferation, aneurysms, diseases characterized by loss of smooth muscle cells or reduced smooth muscle cell proliferation, stroke, ischemia, ulcers, allergies, benign prostatic hypertrophy, migraine, vomiting, psychotic and neurological disorders, including anxiety, schizophrenia, manic depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome and other related diseases including thrombosis and other cardiovascular diseases, autoimmune and inflammatory diseases.

The interaction of GPR7 with an another agonist, such as, for example, L7C can be used as the basis of assays for the diagnosis or monitoring of diseases, disorders or processes involving the signalling of receptors GPR7. Similarly the interaction of GPR8 with an another agonist, such as, for example, L7 can be used as the basis of assays for the diagnosis or monitoring of diseases, disorders or processes involving the signalling of receptors GPR8. Diagnostic assays for GPR7-related and/or GPR8-related diseases or disorders can have several different forms. First, diagnostic assays can measure the amount of receptor (such as, for example, GPR7 and/or GPR8), their genes or mRNA in a sample of tissue. Assays that measure the amount of mRNA encoding the receptor polypeptide also fit into this category. Second, assays can evaluate the qualities of the receptor or the ligand. For example, assays that determine whether an individual expresses a mutant or variant form of GPR7 and/or GPR8, or a polypeptide ligand can be used diagnostically. Third, assays that measure one or more activities of GPR7 and/or GPR8 polypeptide can be used diagnostically.

Assays that Measure the Amount of GPR7 and/or GPR8

GPR7 and/or GPR8 levels can be measured and compared to standards in order to determine whether an abnormal level of the receptor or its ligand is present in a sample, either of which indicate probable dysregulation of GPR7 and/or GPR8 signalling. Polypeptide levels are measured, for example, by immunohistochemistry using antibodies specific for the polypeptide. A sample isolated from an individual suspected of suffering from a disease or disorder characterized by receptor activity such as, for example, GPR7 is contacted with an antibody for said receptor (e.g. anti-GPR7), and binding of the antibody is measured as known in the art (e.g., by measurement of the activity of an enzyme conjugated to a secondary antibody). Similarly, a sample isolated from an individual suspected of suffering from a disease or disorder characterized by receptor activity such as, for example, GPR8 is contacted with an antibody for said receptor (e.g. anti-GPR8), and binding of the antibody is measured as known in the art (e.g., by measurement of the activity of an enzyme conjugated to a secondary antibody).

Another approach to the measurement of GPR7 and/or GPR8 levels uses flow cytometry analysis of cells from an affected tissue. Methods of flow cytometry, including the fluorescent labeling of antibodies specific for, for example, GPR7, are well known in the art. Other approaches include radioimmunoassay or ELISA. Methods for each of these are also well known in the art.

The amount of binding detected is compared to the binding in a sample of similar tissue from a healthy individual, or from a site on the affected individual that is not so affected. An increase of 10% or more relative to the standard is diagnostic for a disease or disorder characterized by GPR7 dysregulation. The test may also be applied to other receptors including, for example, GPR8.

GPR7 and/or GPR8 expression can also be measured by determining the amount of mRNA encoding the polypeptides in a sample of tissue. Levels of mRNA can be measured by quantitative or semi-quantitative PCR. Methods of “quantitative” amplification are well known to those of skill in the art, and primer sequences for the amplification of both GPR7 and GPR8 are disclosed herein. A common method of quantitative PCR involves simultaneously co-amplifying a known quantity of a control sequence using the same primers. This provides an internal standard that can be used to calibrate the PCR reaction. Detailed protocols for quantitative PCR are provided in PCR Protocols A Guide to Methods and Applications, Innis et al., Academic Press, Inc. N.Y., (1990), which is incorporated herein by reference. An increase of 10% or more in the amount of mRNA encoding a receptor such as, for example, GPR7 in a sample, relative to the amount expressed in a sample of like tissue from a healthy individual or in a sample of tissue from an unaffected location in an affected individual is diagnostic for a disease or disorder characterized by dysregulation of GPR7 signalling. Similarly, an increase of 10% or more in the amount of mRNA encoding a receptor such as, for example, GPR8 in a sample, relative to the amount expressed in a sample of like tissue from a healthy individual or in a sample of tissue from an unaffected location in an affected individual is diagnostic for a disease or disorder characterized by dysregulation of GPR8 signalling.

Qualitative GRP7 and GPR8 Assays

Assays that evaluate whether the GPR7 and/or the GPR8 polypeptide or the mRNA encoding them are wild-type or not can be used diagnostically. In order to diagnose a disease or disorder characterized by, for example, GPR7 dysregulation in this manner, RNA isolated from a sample is used as a template for PCR amplification of the receptor. The amplified sequences are then either directly sequenced using standard methods, or are first cloned into a vector, followed by sequencing. A difference in the sequence of one or more encoded amino acids relative to the sequence of wild-type GPR7 can be diagnostic of a disease or disorder characterized by dysregulation of GPR7 signalling. It can be useful, when a change in coding sequence is identified in a sample, to express the variant receptor or ligand and compare its activity to that of wild type receptor (e.g. GPR7). Among other benefits, this approach can provide novel mutants, including constitutively active and null mutants. The above assay may equally well be applied to detect a disease or disorder characterized by GPR8 dysregulation. In addition to standard sequencing methods, amplified sequences can be assayed for the presence of specific mutations using, for example, hybridization of molecular beacons that discriminate between wild type and variant sequences. Hybridization assays that discriminate on the basis of changes as small as one nucleotide are well known in the art. Alternatively, any of a number of “minisequencing” assays can be performed, including, those described, for example, in U.S. Pat. Nos. 5,888,819, 6,004,744 and 6,013,431 (incorporated herein by reference). These assays and others known in the art can determine the presence, in a given sample, of a nucleic acid with a known polymorphism.

If desired, array or microarray-based methods can be used to analyze the expression or the presence of one of more mutations in GPR7 and/or GPR8 sequences. Array-based methods for minisequencing and for quantitation of nucleic acid expression are well known in the art.

Functional GPR7 and GPR8 Assays.

Diagnosis of a disease or disorder characterized by the dysregulation of GPR7 and/or GPR8 signalling can also be performed using functional assays. To do so, cell membranes or cell extracts prepared from a tissue sample are used in an assay of GPR7 activity as described herein (e.g., ligand binding assays, the GTP-binding assay, GTPase assay, adenylate cyclase assay, cAMP assay, arachidonic acid level, phospholipid breakdown, diacyl glycerol or inositol triphosphate assays, PKC activation assay, or kinase assay). The activity detected is compared to that in a standard sample taken from a healthy individual or from an unaffected site on the affected individual. As an alternative, a sample or extract of a sample can be applied to cells expressing GPR7, followed by measurement of the signalling activity of the said receptor relative to a standard sample. A difference of 10% or more in the activity measured in any of these assays, relative to the activity of the standard, is diagnostic for a disease or disorder characterized by dysregulation of receptor signalling involving GPR7.

The above assay may equally well be applied to the detection of a disease or disorder characterized by the dysregulation of GPR8 signalling. To do so, cell membranes or cell extracts prepared from a tissue sample are used in an assay of GPR8 activity as described herein (e.g., ligand binding assays, the GTP-binding assay, GTPase assay, adenylate cyclase assay, cAMP assay, arachidonic acid level, phospholipid breakdown, diacyl glycerol or inositol triphosphate assays, PKC activation assay, or kinase assay). The activity detected is compared to that in a standard sample taken from a healthy individual or from an unaffected site on the affected individual. As an alternative, a sample or extract of a sample can be applied to cells expressing GPR8, followed by measurement of the signalling activity of the said receptor relative to a standard sample. A difference of 10% or more in the activity measured in any of these assays, relative to the activity of the standard, is diagnostic for a disease or disorder characterized by dysregulation of receptor signalling involving GPR8.

Modulation of GPR7 and/or GPR8 Activity in a Cell According to the Invention

The finding that L6L, L6C, L7′, L7′L, L7, L7C, L8, L8L, L8C, ppL7, or ppL8 are ligands of GPR7 and/or GPR8 provides methods of modulating the activity of a receptor polypeptide such as GPR7 and/or GPR8 in a cell. GPR7 activity is modulated in a cell by delivering to that cell an agent that modulates the function of a GPR7 polypeptide. GPR8 activity is modulated in a cell by delivering to that cell an agent that modulates the function of a GPR8 polypeptide. This modulation can be performed in cultured cells as part of an assay for the identification of additional modulating agents, or, for example, in an animal, including a human. Agents include L7C, L7 and their analogues as defined herein, as well as additional modulators identified using the screening methods described herein including but not limited to any of the L7C or L7 analogues.

An agent can be delivered to a cell by adding it to culture medium. The amount to deliver will vary with the identity of the agent and with the purpose for which it is delivered. For example, in a culture assay to identify antagonists of GPR7 activity, one will preferably add an amount of L7C (or another agonist) that half-maximally activates the receptors (e.g., approximately EC₅₀), preferably without exceeding the dose required for receptor saturation. Similarly, in a culture assay to identify antagonists of GPR8 activity, one will preferably add an amount of L7 (or another agonist) that half-maximally activates the receptors (e.g., approximately EC₅₀), preferably without exceeding the dose required for receptor saturation. This dose can be determined for GPR7 receptors by titrating the amount of L7C to determine the point at which further addition of L7C has no additional effect on receptor activity. The dose can be determined for GPR8 receptors by titrating the amount of L7 to determine the point at which further addition of L7 has no additional effect on receptor activity.

When a modulator of GPR7 and/or GPR8 activity is administered to an animal for the treatment of a disease or disorder, the amount administered can be adjusted by one of skill in the art on the basis of the desired outcome. Successful treatment is achieved when one or more measurable aspects of the pathology (e.g., tumor cell growth, accumulation of inflammatory cells) is changed by at least 10% relative to the value for that aspect prior to treatment.

Candidate Modulators Useful According to the Invention

The invention provides for a compound that is a modulator of a receptor of the invention.

The candidate compound may be a synthetic compound, or a mixture of compounds, or may be a natural product (e.g. a plant extract or culture supernatant). A candidate compound according to the invention includes a small molecule that can be synthesized, a natural extract, peptides, proteins, carbohydrates, lipids etc.

Candidate modulator compounds from large libraries of synthetic or natural compounds can be screened. Numerous means are currently used for random and directed synthesis of saccharide, peptide, and nucleic acid based compounds. Synthetic compound libraries are commercially available from a number of companies including Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Combinatorial libraries are available and can be prepared. Alternatively, libraries of natural compounds in the form of bacterial, fungal, plant and animal extracts are available from e.g., Pan Laboratories (Bothell, Wash.) or MycoSearch (NC), or are readily producible by methods well known in the art. Additionally, natural and synthetically produced libraries and compounds are readily modified through conventional chemical, physical, and biochemical means.

Useful compounds may be found within numerous chemical classes. Useful compounds may be organic compounds, or small organic compounds. Small organic compounds have a molecular weight of more than 50 yet less than about 2,500 daltons, preferably less than about 750, more preferably less than about 350 daltons. Exemplary classes include heterocycles, peptides, saccharides, steroids, and the like. The compounds may be modified to enhance efficacy, stability, pharmaceutical compatibility, and the like. Structural identification of an agent may be used to identify, generate, or screen additional agents. For example, where peptide agents are identified, they may be modified in a variety of ways to enhance their stability, such as using an unnatural amino acid, such as a D-amino acid, particularly D-alanine, by functionalizing the amino or carboxylic terminus, e.g. for the amino group, acylation or alkylation, and for the carboxyl group, esterification or amidification, or the like.

For primary screening, a useful concentration of a candidate compound according to the invention is from about 10 μM to about 100 μM or more (i.e. 1 mM, 10 mM, 100 mM, 1 M etc.). The primary screening concentration will be used as an upper limit, along with nine additional concentrations, wherein the additional concentrations are determined by reducing the primary screening concentration at half-log intervals (e.g. for 9 more concentrations) for secondary screens or for generating concentration curves.

Antibodies Useful According to the Invention

The invention provides for antibodies to L6L, L6C, L7′, L7′L, L7, L7C, L8, L8L, L8C, ppL7, or ppL8. Antibodies can also be raised to other specific proteins comprising the consensus sequence WYKxxAxxxxxT/SVGRAAGLLxGL that specifically bind GPR7 and/or GPR8 polypeptides. Antibodies can be made using standard protocols known in the art (see, for example, Antibodies: A Laboratory Manual ed. by Harlow and Lane (Cold Spring Harbor Press: 1988)). A mammal, such as a mouse, hamster, or rabbit can be immunized with an immunogenic form of the peptide (e.g., L7C peptide, L7 peptide or an antigenic fragment which is capable of eliciting an antibody response, or a fusion protein as described herein above). Immunogens for raising antibodies are prepared by mixing the polypeptides (e.g., isolated recombinant polypeptides or synthetic peptides) with adjuvants. Alternatively, L7C peptides (for example) are made as fusion proteins to larger immunogenic proteins. Peptides can also be covalently linked to other larger immunogenic proteins, such as keyhole limpet hemocyanin. Alternatively, plasmid or viral vectors encoding L6L, L6C, L7′, L7′L, L7, L7C, L8, L8L, L8C, ppL7, or ppL8 peptides, or a fragment of these precursor proteins, can be used to express the peptides or the polypeptides and generate an immune response in an animal as described in Costagliola et al., 2000, J. Clin. Invest. 105:803-811, which is incorporated herein by reference. In order to raise antibodies, immunogens are typically administered intradermally, subcutaneously, or intramuscularly to experimental animals such as rabbits, sheep, and mice. In addition to the antibodies discussed above, genetically engineered antibody derivatives can be made, such as single chain antibodies.

The progress of immunization can be monitored by detection of antibody titers in plasma or serum. Standard ELISA, flow cytometry or other immunoassays can also be used with the immunogen as antigen to assess the levels of antibodies. Antibody preparations can be simply serum from an immunized animal, or if desired, polyclonal antibodies can be isolated from the serum by, for example, affinity chromatography using immobilized immunogen.

To produce monoclonal antibodies, antibody-producing splenocytes can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with L6L, L6C, L7′, L7′L, L7, L7C, L8, L8L, L8C, ppL7, and/or ppL8 peptides, and monoclonal antibodies isolated from the media of a culture comprising such hybridoma cells.

High Throughput Screening Kit

A high throughput screening kit according to the invention comprises all the necessary means and media for performing the detection of a modulator compound including an agonist, antagonist, inverse agonist or inhibitor to the receptor of the invention in the presence of an agonist, such as for example, L7C, preferably at a concentration in the range of 1 μM to 1 mM. The kit comprises the following successive steps. Recombinant cells of the invention, comprising and expressing the nucleotide sequence encoding the receptor, for example, GPR7, are grown on a solid support, such as a microtiter plate, more preferably a 96 well microtiter plate, according to methods well known to the person skilled in the art especially as described in WO 00/02045. Modulator compounds according to the invention, at concentrations from about 1 μM to 1 mM or more, are added to the culture media of defined wells in the presence of an appropriate concentration of agonist, such as, for example, L7C, said concentration of L7C preferably in the range of 1 μM to 1 μM.

Alternatively, a high throughput screening kit according to the invention comprises all the necessary means and media for performing the detection of a modulator compound including an agonist, antagonist, inverse agonist or inhibitor to the receptor of the invention in the presence of an agonist, such as for example, L7, preferably at a concentration in the range of 1 μM to 1 mM. The kit comprises the following successive steps. Recombinant cells of the invention, comprising and expressing the nucleotide sequence encoding the receptor, for example, GPR8, are grown on a solid support, such as a microtiter plate, more preferably a 96 well microtiter plate, according to methods well known to the person skilled in the art especially as described in WO 00/02045. Modulator compounds according to the invention, at concentrations from about 1 μM to 1 mM or more, are added to the culture media of defined wells in the presence of an appropriate concentration of agonist, such as, for example, L7, said concentration of L7 preferably in the range of 1 μM to 1 μM.

Secondary messenger assays, amenable to high throughput screening analysis, are performed including but not limited to the measurement of intracellular levels of cAMP, intracellular inositol phosphate, intracellular diacylglycerol concentrations, arachinoid acid concentration or MAP kinase or tyrosine kinase activity (as decribed above). For example, the GPR7 activity, as measured in a cyclic AMP assay, is quantified by a chemiluminescence assay (cAMP-Screen 96-Well. Chemiluminescent Immunoassay System ref: CS1000 kit, Applied Biosystem, USA). Results are compared to the baseline level of GPR7 activity obtained from recombinant cells according to the invention in the presence of L7C but in the absence of added modulator compound. Wells showing at least 2 fold, preferably 5 fold, more preferably 10 fold and most preferably a 100 fold or more increase or decrease in GPR7 activity as compared to the level of activity in the absence of modulator, are selected for further analysis. Secondary messenger assays may also be performed using an alternative combination of receptor and ligand such as GPR8 and L7.

Other Kits Useful According to the Invention

The invention provides for kits useful for screening for modulators of activity of receptors such as, for example, GPR7 and GPR8, as well as kits useful for diagnosis of diseases or disorders characterized by dysregulation of GPR7 and/or GPR8 signalling. Kits useful according to the invention can include an isolated GPR7 polypeptide (including a membrane-or cell-associated GPR7 polypeptide, such as that found on isolated membranes, found in cells expressing GPR7, or, found on an SPR chip). A kit can also comprise an antibody specific for GPR7. Alternatively, or in addition, a kit can contain cells transformed to express GPR7 polypeptide. In a further embodiment, a kit according to the invention can contain a polynucleotide encoding a GPR7 polypeptide. In a still further embodiment, a kit according to the invention may comprise the specific primers useful for amplification of GPR7 as described below

Other kits useful according to the invention can include an isolated GPR8 polypeptide (including a membrane-or cell-associated GPR8 polypeptide, such as that found on isolated membranes, found in cells expressing GPR8, or, found on an SPR chip). A kit can also comprise an antibody specific for GPR8. Alternatively, or in addition, a kit can contain cells transformed to express GPR8 polypeptide. In a further embodiment, a kit according to the invention can contain a polynucleotide encoding a GPR8 polypeptide. In a still further embodiment, a kit according to the invention may comprise the specific primers useful for amplification of GPR8 as described below.

Kits according to the invention might comprise the stated items or combinations of items and packaging materials therefor. Kits might also include instructions for use.

Transgenic Animals

Transgenic mice provide a useful tool for genetic and developmental biology studies and for the determination of the function of a novel sequence. According to the method of conventional transgenesis, additional copies of normal or modified genes are injected into the male pronucleus of the zygote and become integrated into the genomic DNA of the recipient mouse. The transgene is transmitted in a Mendelian manner in established transgenic strains. Constructs useful for creating transgenic animals comprise genes under the control of either their normal promoters or an inducible promoter, reporter genes under the control of promoters to be analyzed with respect to their patterns of tissue expression and regulation, and constructs containing dominant mutations, mutant promoters, and artificial fusion genes to be studied with regard to their specific developmental outcome. Typically, DNA fragments on the order of 10 kilobases or less are used to construct a transgenic animal (Reeves, 1998, New. Anat., 253:19). Transgenic animals can be created with a construct comprising a candidate gene containing one or more polymorphisms according to the invention. Alternatively, a transgenic animal expressing a candidate gene containing a single polymorphism can be crossed to a second transgenic animal expressing a candidate gene containing a different polymorphism and the combined effects of the two polymorphisms can be studied in the offspring animals.

Other Transgenic Animals

The invention provides for transgenic animals that include but are not limited to transgenic mice, rabbits, rats, pigs, sheep, horses, cows, goats, etc. A protocol for the production of a transgenic pig can be found in White and Yannoutsos, Current Topics in Complement Research: 64^{th} Forum in Immunology, pp. 88-94; U.S. Pat. No. 5,523,226; U.S. Pat. No. 5,573,933: PCT Application No. WO93/25071; and PCT Application No. WO95/04744. A protocol for the production of a transgenic mouse can be found in U.S. Pat. No. 5,530,177. A protocol for the production of a transgenic rat can be found in Bader and Ganten, Clinical and Experimental Pharmacology and Physiology, Supp. 3:S81-S87, 1996. A protocol for the production of a transgenic cow can be found in Transgenic Animal Technology, A Handbook, 1994, ed., Carl A. Pinkert, Academic Press, Inc. A protocol for the production of a transgenic rabbit can be found in Hammer et al., Nature 315:680-683, 1985 and Taylor and Fan, Frontiers in Bioscience 2:d298-308, 1997.

Knock out Animals

i) Standard

Knock out animals are produced by the method of creating gene deletions with homologous recombination. This technique is based on the development of embryonic stem (ES) cells that are derived from embryos, are maintained in culture and have the capacity to participate in the development of every tissue in the mouse when introduced into a host blastocyst. A knock out animal is produced by directing homologous recombination to a specific target gene in the ES cells, thereby producing a null allele of the gene. The potential phenotypic consequences of this null allele (either in heterozygous or homozygous offspring) can be analyzed (Reeves, supra).

ii) In vivo Tissue Specific Knock out in Mice using Cre-lox.

The method of targeted homologous recombination has been improved by the development of a system for site-specific recombination based on the bacteriophage P1 site specific recombinase Cre. The Cre-loxP site-specific DNA recombinase from bacteriophage P1 is used in transgenic mouse assays in order to create gene knockouts restricted to defined tissues or developmental stages. Regionally restricted genetic deletion, as opposed to global gene knockout, has the advantage that a phenotype can be attributed to a particular cell/tissue (Marth, 1996, Clin. Invest. 97: 1999). In the Cre-loxP system one transgenic mouse strain is engineered such that loxP sites flank one or more exons of the gene of interest. Homozygotes for this so called ‘floxed gene’ are crossed with a second transgenic mouse that expresses the Cre gene under control of a cell/tissue type transcriptional promoter. Cre protein then excises DNA between loxP recognition sequences and effectively removes target gene function (Sauer, 1998, Methods, 14:381). There are now many in vivo examples of this method, including the inducible inactivation of mammary tissue specific genes (Wagner et al., 1997, Nucleic Acids Res., 25:4323).

iii) Bac Rescue of Knock Out Phenotype

In order to verify that a particular genetic polymorphism/mutation is responsible for altered protein function in vivo one can “rescue” the altered protein function by introducing a wild-type copy of the gene in question. In vivo complementation with bacterial artificial chromosome (BAC) clones expressed in transgenic mice can be used for these purposes. This method has been used for the identification of the mouse circadian Clock gene (Antoch et al., 1997, Cell 89: 655).

Dosage and Mode of Administration

By way of example, a patient can be treated as follows by the administration of a modulator of GPR7 (for example, an agonist, antagonist or inhibitor of GPR7, of the invention). A modulator of GPR7 found by the invention can be administered to the patient, preferably in a biologically compatible solution or a pharmaceutically acceptable delivery vehicle, by ingestion, injection, inhalation or any number of other methods. The dosages administered will vary from patient to patient; a “therapeutically effective dose” can be determined, for example but not limited to, by the level of enhancement of function (e.g., as determined in a second messenger assay described herein). Monitoring L7C (or another ligand) binding might also enable one skilled in the art to select and adjust the dosages administered. The dosage of a modulator of GPR7 of the invention may be repeated daily, weekly, monthly, yearly, or as considered appropriate by the treating physician.

By way of another example, a patient can be treated as follows by the administration of a modulator of GPR8 (for example, an agonist, antagonist or inhibitor of GPR8, of the invention). A modulator of GPR8 found by the invention can be administered to the patient, preferably in a biologically compatible solution or a pharmaceutically acceptable delivery vehicle, by ingestion, injection, inhalation or any number of other methods. The dosages administered will vary from patient to patient; a “therapeutically effective dose” can be determined, for example but not limited to, by the level of enhancement of function (e.g., as determined in a second messenger assay described herein). Monitoring L7 (or another ligand) binding might also enable one skilled in the art to select and adjust the dosages administered. The dosage of a modulator of GPR8 of the invention may be repeated daily, weekly, monthly, yearly, or as considered appropriate by the treating physician.

Pharmaceutical Compositions

The invention provides for compositions comprising a GPR7 modulator according to the invention admixed with a physiologically compatible carrier. The invention also provides for compositions comprising a GPR8 modulator according to the invention admixed with a physiologically compatible carrier. As used herein, “physiologically compatible carrier” refers to a physiologically acceptable diluent such as water, phosphate buffered saline, or saline, and further may include an adjuvant. Adjuvants such as incomplete Freund's adjuvant, aluminium phosphate, aluminium hydroxide, or alum are materials well known in the art.

The invention also provides for pharmaceutical compositions. In addition to the active ingredients, these pharmaceutical compositions may contain suitable pharmaceutically acceptable carrier preparations which can be used pharmaceutically.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer' solution, or physiologically buffered, saline. Aqueous injection suspensions may contain substances which increase the viscosity of the suspension, such as sodium carboxymethyl cellulose, sorbitol, or dextran. Additionally, suspensions of the active solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1% to 2% sucrose, 2% to 7% mannitol at a pH range of 4.5 to 5.5 that is combined with buffer prior to use.

After pharmaceutical compositions comprising a compound of the invention formulated in a acceptable carrier have been prepared, they can be placed in an appropriate container and labeled for treatment of an indicated condition with information including amount, frequency and method of administration.

All references referred to below and above are incorporated by reference in their entirety.

### BRIEF DESCRIPTION OF FIGURES

FIG. 1: (1A) represents nuleotide (SEQ ID NO: 24) and deduced amino acid (SEQ ID NO: 23) sequence of the human GPR7 receptor according to the invention. (1B) represents the 7 putative transmembrane domains of the GPR7 receptor; these domains are underlined.

FIG. 2: (2A) Alignment of GPR7 and GPR8 amino acid sequences, using ClustalX. 62% identity between GPR7 and GPR8. (2B) Alignment of GPR7 (U22491) and GPR8 (U22492) nucleotide sequences, using ClustalX. 71% identity between GPR7 and GPR8. (2C) is a dendrogram representing the structural similarity between the GPR7 receptors and related receptors.

FIG. 3: represents nucleotide and deduced amino acid sequences of the human GPR7 and human GPR8 and of human L6, L6L, L6C, L7′, L7′L, L7, L7AL, L7C, L8, L8L, L8C, ppL7, ppL8 and ligands according to the invention.

FIGS. 4 A, B: Distribution of the human GPR7 receptor in tissue. RT-PCR experiments were carried out using a panel of poly A+ RNA (Clontech and Ambion). Total RNA was prepared from blood cells and cell lines. The expected size of the amplified GPR7 and GAPDH bands were 746 and 509 bp, respectively. cDNA (−) indicates negatives controls of the PCR reaction without cDNA template. Aliquots (10 μl) of the PCR reaction were analyzed by gel electrophoresis.

FIG. 5: illustrates the activity of L7 and L8 on the luminescence emission of CHO—K1 cells stably expressing the human GPR7, mitochondrial apoaequorin and Gα16.

FIG. 6: illustrates the activity of L7′ and L8 on the accumulation of GTPγ[³⁵S] bound to a membrane preparation from COS-7 cells transiently transfected with the human GPR7 or expression plasmid alone (negative control).

FIG. 7: illustrates the effect of L7 and L8 on cAMP accumulation in CHO—WTA11 cells expressing GPR7

FIG. 8: Distribution of GPR7, GPR8, ppL7 and ppL8 transcripts in the human central nervous system. RT-PCR experiments were carried out using a panel of total and polyA+ RNA and specific primers for GPR7, GPR8, ppL7 and ppL8 sequences, as described in Example 7. The expected size of the amplified bands were 746 bp, 898 bp, 189 bp and 377 bp for GPR7, GPR8, ppL7 and ppL8 respectively. Aliquots (10 μl) of the PCR reactions were analysed by 1% agarose gel electrophoresis. Amplification of GAPDH (509 bp) transcripts was performed in parallel as a control.

FIG. 9: Pharmacology of human GPR7 and GPR8. Aequorin-based functional assay using WTA11 cells expressing GPR7 (panel A) and GPR8 (panel B). Competition binding assay using either membranes of CHO—K1 cells expressing GPR7 and [1251]-L7 as tracer (panel C) or membranes of CHO-K1 cells expressing GPR8 and [1251]-L8 as tracer (panel D) using L7, L7C, L8 and L8C as competitors. The displayed curves are representative of at least three independent experiments. The data represent the mean and S.E.M. of triplicate data points.

FIG. 10: cAMP accumulation in CHO—K1 cells expressing GPR7 or GPR8. Cell lines expressing GPR7 (panel A) or GPR8 (panel B) were incubated in the presence of the various concentrations of L7, L7C, L8 and L8C, together with 5 μM forskolin. The displayed curves are representative of at least three independent experiments. The data represent the mean and S.E.M. of triplicate data points.

FIG. 11: Structure of L7 and L8 peptides, precursors and genes. Panel A, nucleotide and deduced amino acid sequence of human L7 precursor cDNA. The diarginine motives are double underlined. The sequence of L7 (which stops at the first diarginine motive) and L7C (which stops at the second diarginine motive) is underlined. Dash line corresponds to the sequence of the intron. Panel B, comparison of amino acid sequences of the pre-pro-peptides for L7 and L8. Residues identical for all precursors are shaded in black; Residues identical only for the L7 precursors in dark grey; Residues identical only for L8 precursors in light grey. The arrow indicates the first residue after cleavage of the predicted signal peptides. The black dots indicate the localization of the two diarginine motives (only one for the mouse ppL7) that may serve as cleavage signal of the propeptides. The human L7 precursor (h-ppL7) sequence was deduced from the sequence of the cDNA we cloned by PCR. The mouse L7 precursor (m-ppL7) sequence was deduced from the sequence of the EST (GenBank #BB655095). The human (h), mouse (m), rat (r) and pig (p) L8 precursor sequences are from the patent application WO 01/98494 A1.

### EXAMPLES

The invention is illustrated by the following nonlimiting examples wherein the following materials and methods are employed. The entire disclosure of each of the literature references cited hereinafter are incorporated by reference herein.

Materials

Trypsin was from Flow Laboratories (Bioggio, Switzerland). Culture media, G418, fetal bovine serum (FBS), restriction enzymes, Platinum Pfx and Taq DNA polymerases were purchased from Life Technologies, Inc. (Merelbeke, Belgium). The radioactive product myo-D-[2-³H]inositol (17.7 Ci/mmol) was from Amersham (Ghent, Belgium). Dowex AG1X8 (formate form) was from Bio-Rad Laboratories (Richmond, Calif.). ATP, was obtained from Sigma Chemical Co. (St. Louis, Mo.). L6, L6L, L6C, L7′, L7′L, L7, L7AL, L7C, L8, L8L and L8C were synthesised by Eurogentec, Belgium. Forskolin was purchased from Calbiochem. (Bierges, Belgium). Rolipram was a gift from the Laboratories Jacques Logeais (Trappes, France).

### Example 1

Cloning of GPR7, Sequencing and Alignment

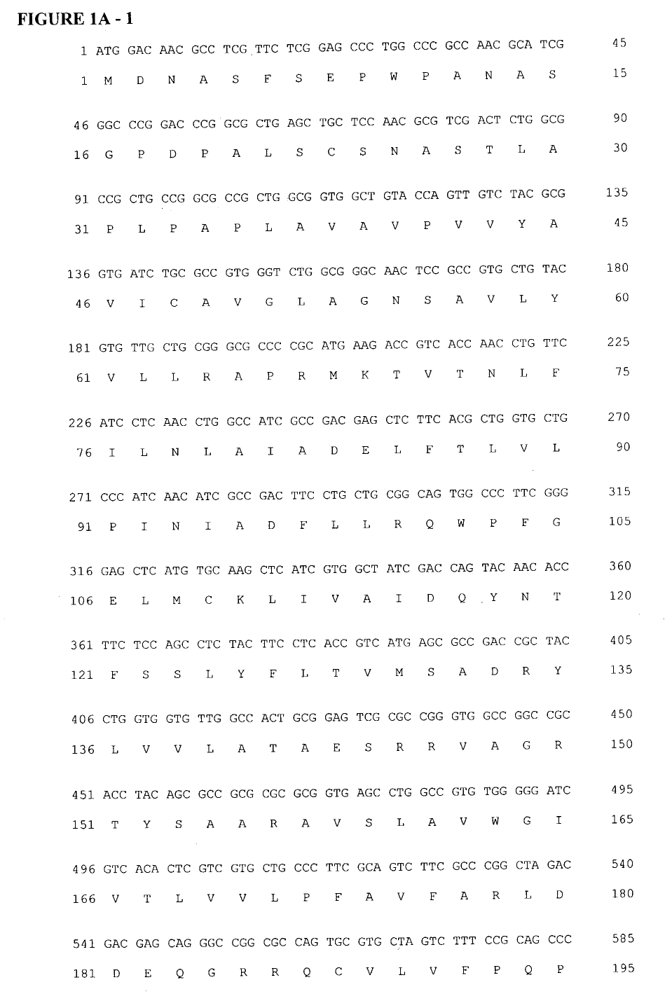

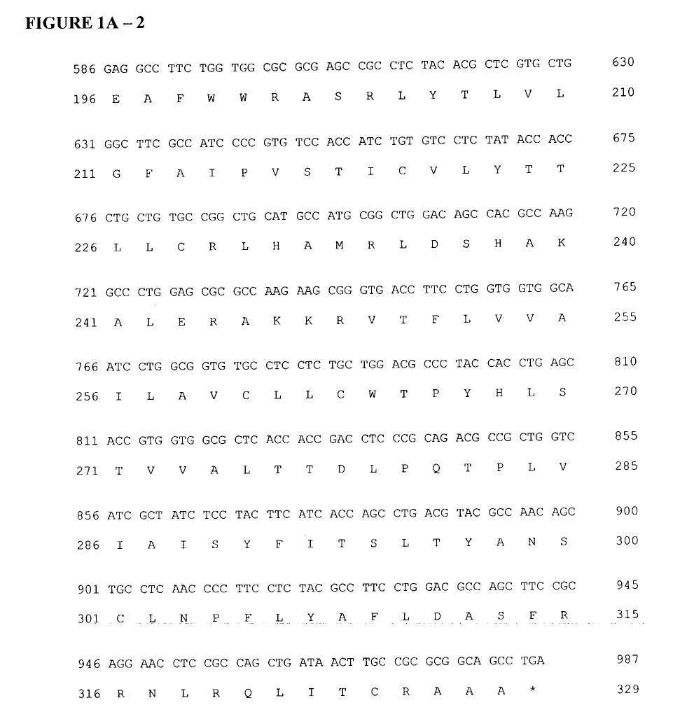

Specific oligonucleotide primers were synthesized on the basis of the sequence of the GPR7 human receptor: a sense primer 5′-CCGGGATCCACCATGGACAACGCCTCGTTCTCG-3′ and an antisense primer 5′-CTAGTCTAGATCAGGCTGCCGCGCGGCAAGT-3′. A polymerase chain reaction (PCR) was performed on genomic DNA using the Pfu DNA Polymerase. The amplification conditions were as follows: 94° C., 15 s; 50° C., 30 s; 72° C., 1 min for 35 cycles. Amplifications resulted in a fragment of 0.98 kilobase containing the entire coding sequence of the GPR7 gene. The coding sequence was then subdloned between the BamHI and XbaI sites of expression vector pcDNA3 (Invitrogen) and sequenced on both strands for each of the three cDNAs using the BigDye Terminator cycle sequencing kit (Applied Biosystems, Warrington, Great Britain) (FIG. 1a). The seven putative membrane-spanning domains are underlined (FIG. 1b).

This 984 base pairs (bp)-open reading frame was also identified by O'Dowd et al. (GenBank accession U22491) and reported to encode an orphan G-protein-coupled receptor that they called GPR7. Oligonucleotide primers were synthesized on the basis of this coding sequence published in O'Dowd et al. (1995).

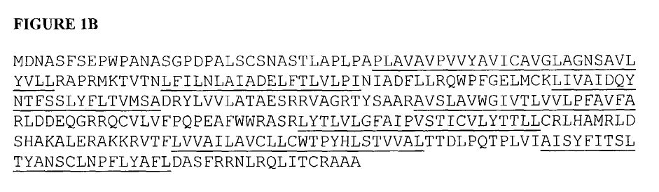

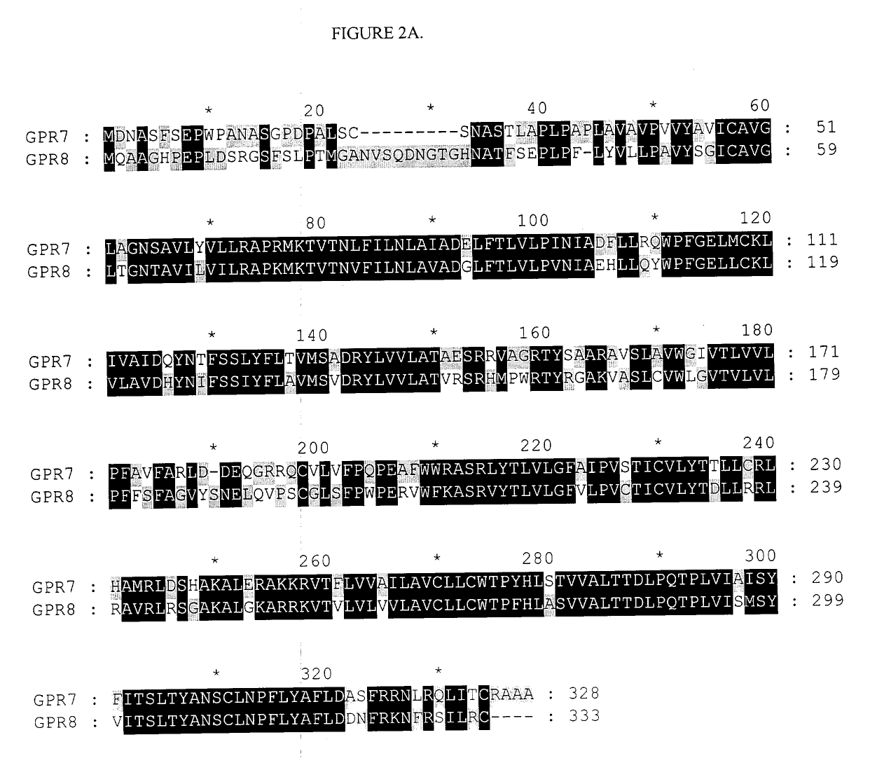

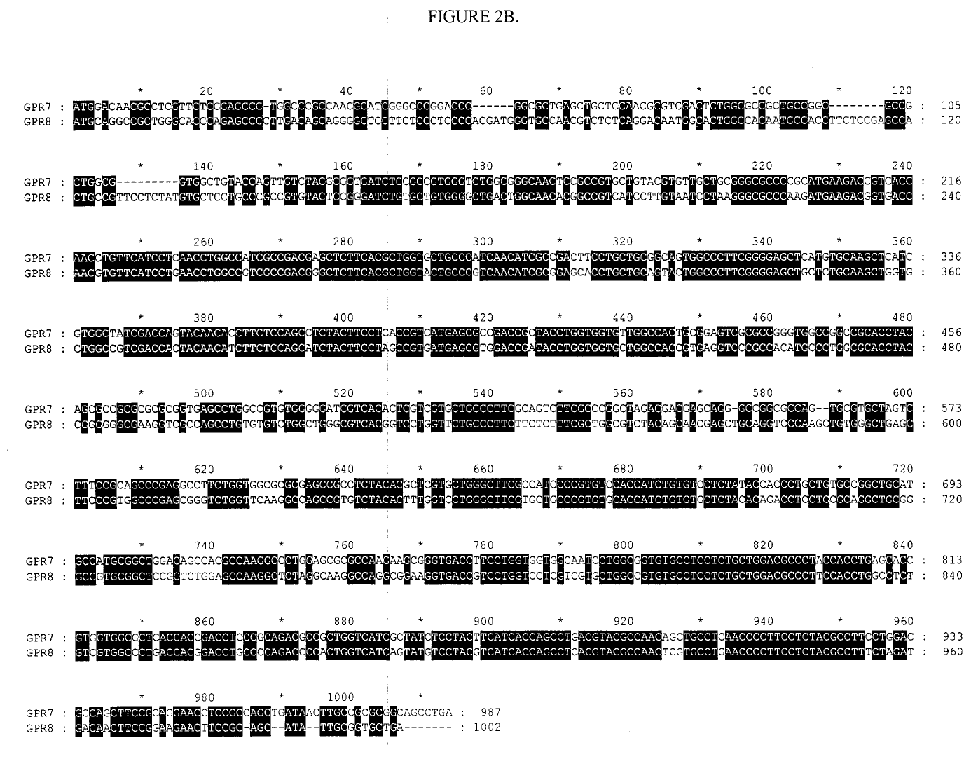

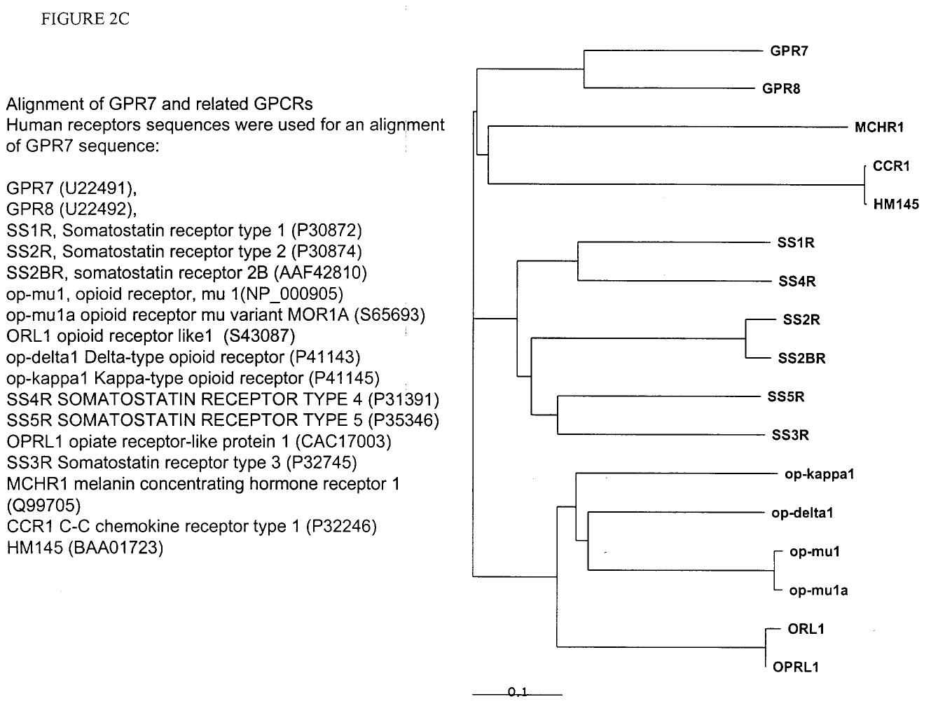

Alignment of the amino acid sequences (FIG. 2a) and nucleotidic sequences (FIG. 2b) of GPR7 and GPR8. Alignment of the amino acid sequences of GPR7 and GPR8 (FIG. 2c) with related sequences were performed using ClustalX algorithm. Then, the dendrogram was constucted using TreeView algorithm.

### Example 2

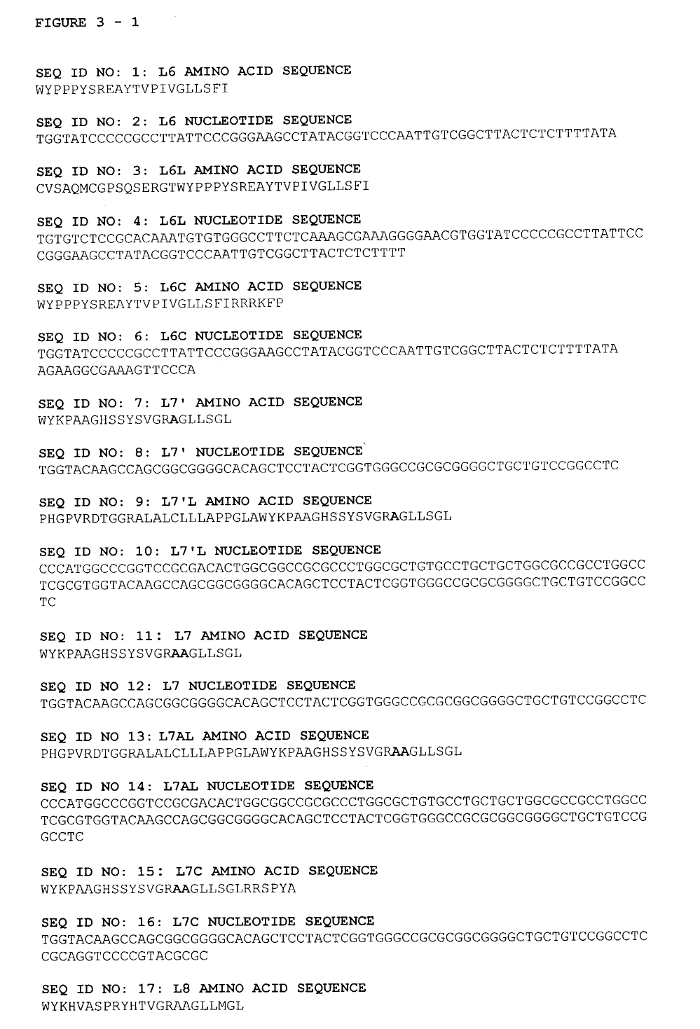

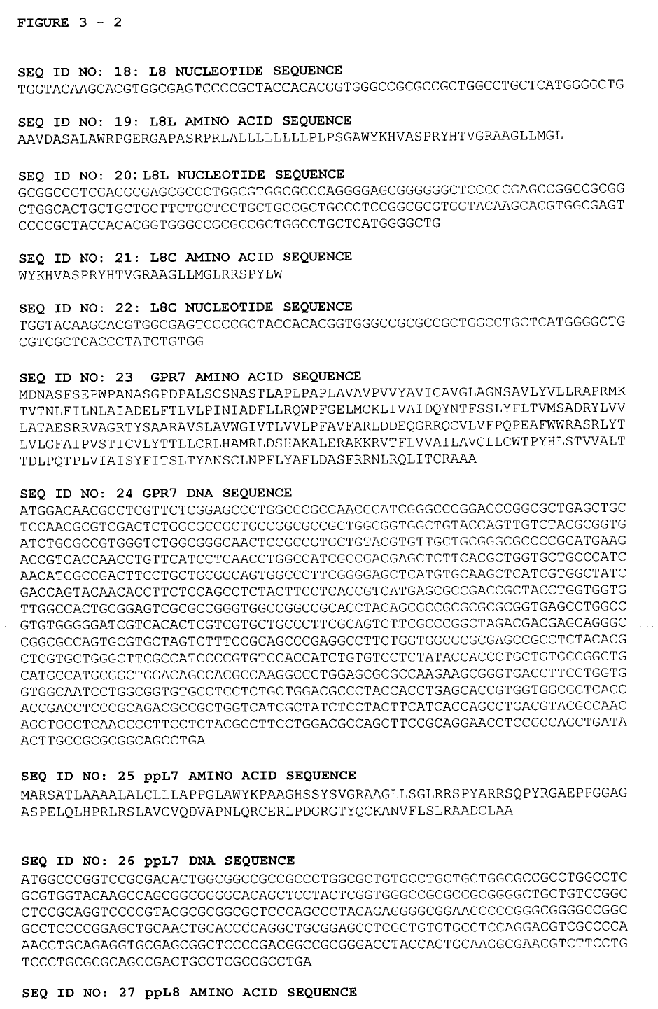

From the sequence of GPR8 ligand (L8) disclosed in PCT application no. WO 01/98494 we performed TBLASTN searches in several databases. Several hits were found with amino acid sequences closely related to L8. We called these sequences L6, L6L, L6C L7′, L7′L, L7, L7AL and L7C according to their length and their composition (FIG. 3) These sequences were synthetised by Eurogentec, Belgium and tested with GPR7 receptor in a functional assay.

### Example 3

Tissue Distribution of GPR7 Human Receptor

GPR7 mRNA was amplified by RT-PCR in several human tissues (FIG. 4a and 4b).

Reverse transcription-polymerase chain reaction (RT-PCR) experiments were carried out using a panel of polyA⁺ RNA (Clontech and Ambion). Total RNA from cell lines were prepared with RNAeasy mini kit (Qiagen). The GPR7 primers were as follows: GPR7 sense primer (5′-CTTGGAGAGCTGGAAACGAG-3′) and GPR7 antisense primer (5′-GGACACAGATGGTGGACACG-3′). The expected size of the amplified DNA band was 746 bp. Two primers synthesized on the basis of GAPDH coding sequence were used as controls to produce a product with an expected size of 509 bp: GAPDH sense primer 5′-ACCACCATGGAGAAGGCTGG-3′ and GAPDH antisense reverse 5′-CTCAGTGTAGCCCAGGATGC -3′. Approximately 50 ng of poly A⁺ RNA or 500 ng of total RNA was reverse transcribed with Superscript II (Life Technologies, Inc., Merelbeke, Belgium) and used for PCR. PCR was performed using the Taq polymerase under the following conditions: denaturation at 94° C. for 3 min, 34 cycles at 94° C. for 1 min, 60° C. for 2 min and 72° C. for 50 seconds. Aliquots (10 μl) of the PCR reaction were analysed by 1% agarose gel electrophoresis.

A 746 bp-band was clearly detected in trachea, calu-3 (serous cells of lung adenocarcinoma), pituitary, fetal brain, hippocampus and amygdala. A faint band was observed in brain, thalamus, testis, prostate, small intestine, colon, rectum, lung carcinoma, 6CFSMEo- (airway submucosal gland), skin, fetal spleen,. No signal was detected in spinal cord, cerebellum, caudate nucleus, substancia nigra, corpus callosum, thymus, pancreas, uterus, placenta, stomach, liver, lung, fetal lung, 16HBE14o- (airway bronchoepithelial cells), HASMSC1 (airway smooth muscle), fetal liver, spleen, heart, bladder, kidney, fetal kidney, skeletal muscle, adrenal gland, ovary, thyroid, lymph node, lymphoblastic leukemia, and colorectal adenocarcinoma. The amplification of a fragment of GAPDH coding sequence was used as control.

### Example 4

Activity of L7 and L8 on the Luminescence Emission of CHO—K1 Cells Stably Expressing the Human GPR7

CHO—K1 cells (ATCC CRL-9618 (Bethesda, Md., USA)) were previously transfected with a plasmid encoding mitochondrial apoaequorin and Gα16. A clone obtained by limit dilution, called WTA11, was grown in Nutrient Mixture HAM's F12 medium supplemented with 10% fetal calf serum, 250 μg/ml zeocin, 100 units/ml penicillin and 100 μg/ml streptomycin. A bicistronic plasmid encoding the human GPR7 was transfected into WTA11 CHO—K1 cells, using Fugene 6 (Roche Diagnostics, Mannheim, Germany). Individual clones were selected two days after transfection with 400 μg/ml neomycin and GPR7-positive clones were confirmed by northern blotting, RT-PCR and sequencing.

CHO—K1 cells transfected with the bicistronic plasmid that does not encode the human GPR7 were used as control cells (mock-transfected).

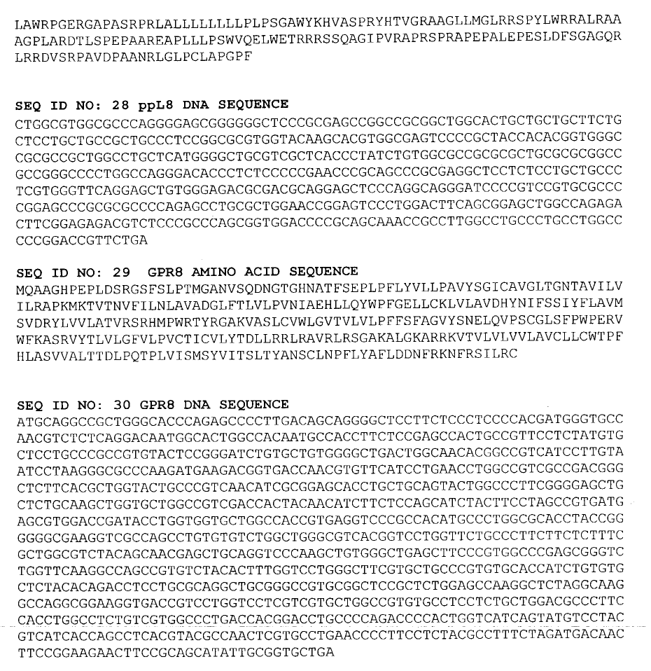

Functional responses were analysed by recording the luminescence of aequorin following the addition of the ligand. In brief, cells were collected from plates with PBS containing 5 mM EDTA, pelleted, and resuspended at 107 cells/ml in DMEM-F12 medium and incubated with 5 μM coelenterainze H (Molecular Porbes) for 4 h at room temperature. Cells were then washed in DMEM-F12 medium and resuspended at a concentration of 2×10⁶ cells/ml. Cells where then mixed with the ligand, and the light emission was recorded over 60 s using a Microlumat™ luminometer (Perkin Elmer). Results are expressed as relative light units (RLU) (FIG. 5).

### Example 5

Activity of L7′ and L8 on the Accumulation of GTPγ[³⁵S]

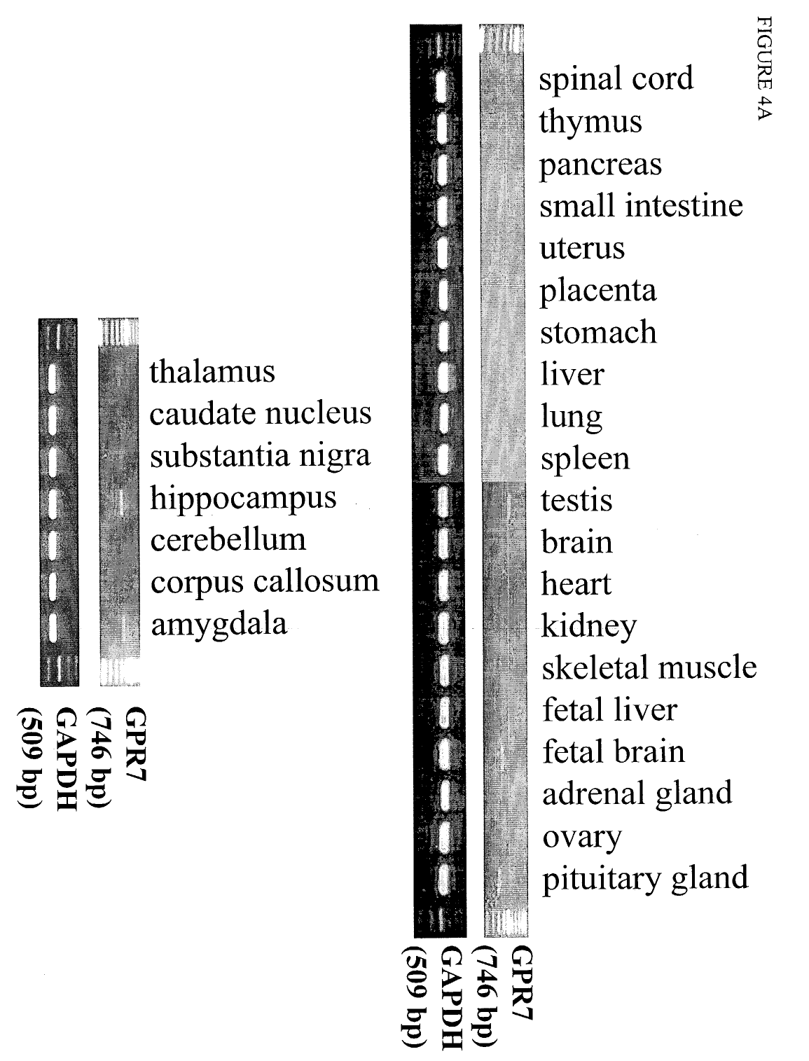

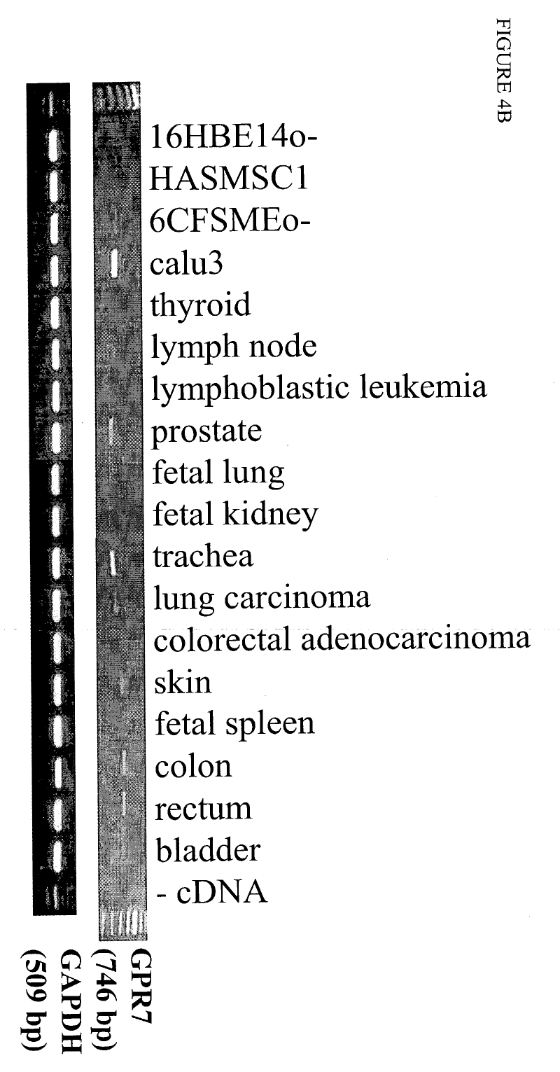

COS-7 cells were transiently transfected with GPR7 or plasmid alone (negative control) using Lipofectamine 2000 (InVitroGen). Two days after transfection, cells were harvested in PBS buffer, frozen at −20° C. for 60 min, and homogenized in 50 mM Tris-HCl, pH7.4, in a tissue grinder. The nuclear pellet was discarded after centrifugation at 1000×g for 15 min at 4° C. and the membrane fraction was collected by centrifugation of the supernatant at 1000,000×g for 30 min at 4° C. Membranes (15 μg) were used for each point. Membranes were incubated in 200 μl solution containing 2 mM HEPES pH7.4, 10 mM NaCl, 3 mM MgCl₂, 3 mM GDP, 10 μg/ml saponin, 0.1 nM GTPγ[³⁵S](1086 Ci/mmol, New England Nuclear, Boston, Mass., USA) and various concentrations of L7′ and L8, at 30° C. for 30 min. The membranes were collected by centrifugation at 1000×g for 10 min at 4° C., and bound GTPγ[³⁵S] was counted (FIG. 6)

### Example 6

Effect of L7 and L8 on cAMP Accumulation in CHO—WTA11 Cells Expressing GPR7

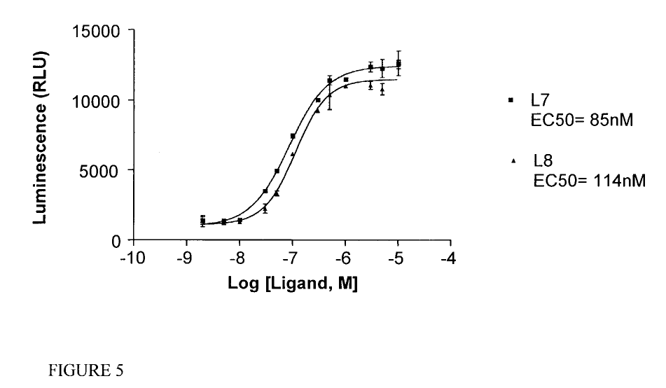

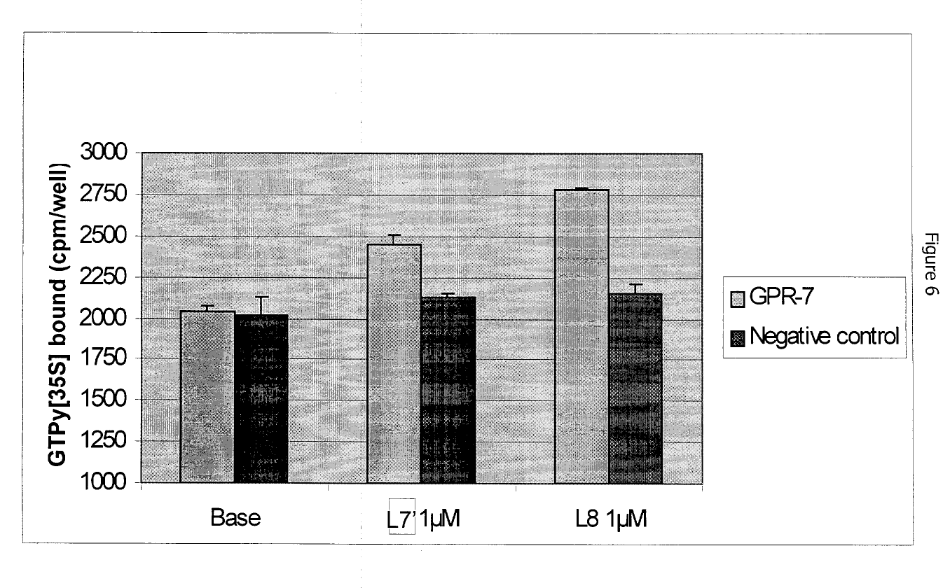

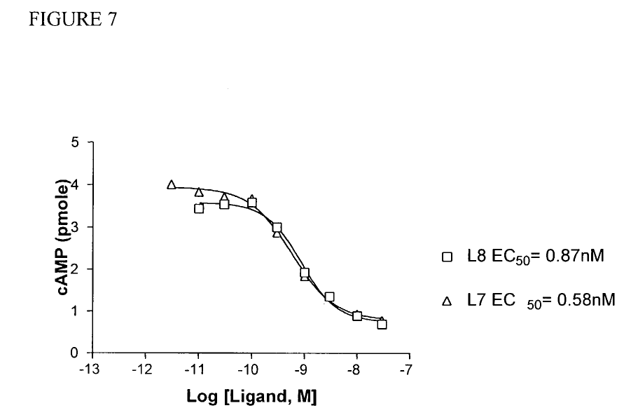

L7 and L8 were tested on CHO—WTA11 cells stably expressing the human GPR7 for their ability to inhibit the activity of the adenylate cyclase stimulated with forskolin. EC₅₀ were similar for both ligands (FIG. 7). There was no effect of L7 or L8 in cells coexpressing another receptor and used as negative control. No stimulation of cAMP production was observed in GPR7 expressing cells incubated with the ligands, in absence of forskolin.

### Example 7

Tissue Distribution of GPR7 and GPR8 Human Receptors and ppL7 and ppL8 Precursors.

To determine the tissue distribution of GPR7 and GPR8 human receptors, reverse transcription-polymerase chain reaction (RT-PCR) experiments were carried out using a panel of polyA+ RNA (Clontech, Palo Alto, Calif., USA and Ambion Austin, Tex., USA). The GPR7 primers were 5′-CTTGGAGAGCTGGAAACGAG-3′ (forward) and 5′-GGACACAGATGGTGGACACG-3′ (reverse), with an expected product size of 746 bp. The GPR8 primers were 5′-GCCACTGCCGTTCCTCTAT-3′ (forward) and 5′-GATGATGGGGGTGATGATGG-3′ (reverse), with an expected product size of 898 bp. A GAPDH cDNA fragment (509 bp) was amplified as control, using as primers 5′-ACCACCATGGAGAAGGCTGG-3′ (forward) and 5′-CTCAGTGTAGCCCAGGATGC-3′ (reverse). Approximately 50 ng of poly A+ RNA or 500 ng of total RNA was reverse transcribed with Superscript II (Life Technologies, Merelbeke, Belgium) and used for PCR. PCR was performed using the Taq polymerase under the following conditions: denaturation at 94° C. for 3 min, 34 cycles at 94° C. for 1 min, 60° C. for 2 min and 72° C. for 50 seconds. Aliquots (10 μl) of the PCR reactions were analysed by 1% agarose gel electrophoresis. To determine the tissue distribution of ppL7 and ppL7 human precursors, prepropeptides ppL7 and ppL8 transcripts were detected by RT-PCR using as primers (Eurogentec, Belgium) 5′-ACAGCTCCTACTCGGTG-3′ (ppL7 forward), 5′-GCACCTTTGCAGGTTTGG-3′ (ppL7 reverse), 5′-CTCCACTGCGCGCCCAAAC-3′ (ppL8 forward) and 5′-GCGTCTGCCACAGCTCCTG-3′ (ppL8 reverse). The expected size of the amplified DNA bands was 189 bp and 377 bp for ppL7 and ppL8, respectively. PCR reactions were performed using Taq DNA polymerase under the following conditions: 94° C. for 5 min; 94° C. for 1 min, 53° C. for 1 min 30, 72° C. for 30 sec, 34 cycles (for ppL7) or 94° C. for 5 min; 94° C. for 1 min, 61° C. for 1 min 30, 72° C. for sec, 29 cycles (for ppL8). Aliquots (10 μl) of the PCR reactions were analysed by 1% agarose gel electrophoresis. Negative controls included a PCR made with no cDNA, and reactions performed with RNA of every tissue sample in the absence of reverse transcription. All controls were negative (data not shown).

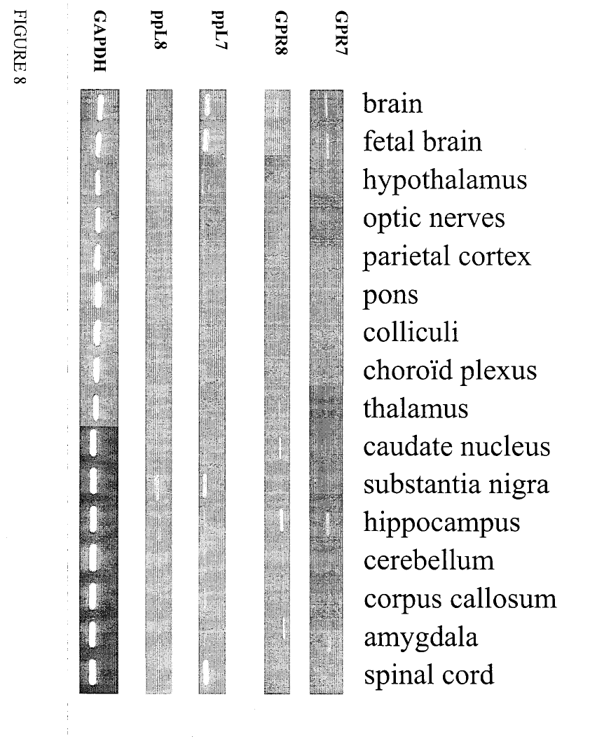

Tissue distribution of the ligand and receptor transcripts was determined by RT-PCR in peripheral tissues and central nervous system regions (FIG. 8 and Table I). For the ppL7 precursor transcript, the expected size of the amplified band was 189 bp. A band of this size was indeed obtained in several tissues, sequenced and found to correspond to encode ppL7. Specific ppL7 transcripts were found at high levels in adult and fetal whole brain, substantia nigra, spinal cord, placenta and colorectal adenocarcinoma (for extended distribution see Table I). A second band of 289 bp was also observed following ppL7 amplification. This band was sequenced and found to correspond to an unspliced form of ppL7 transcript, similar to that found in the ESTs A1394669 and AW058203 of the public databases. This unspliced form of ppL7 transcripts was only detected at low levels (data not shown). Transcripts encoding the ppL8 precursors were detected at high levels by RT-PCR, in the substantia nigra, lymphoblastic leukemia, fetal kidney, colorectal adenocarcinoma and trachea (Table I).

Tissue distribution of the receptor transcripts was determined in parallel. Transcripts encoding GPR7 were detected at high levels in hippocampus, amygdala, trachea and lung carcinoma (Table I). GPR7 transcripts were also detected at moderate levels in fetal brain, pituitary gland and prostate (Table I). GPR8 transcripts were detected at high levels in caudate nucleus, hippocampus, amygdala, and at moderate levels in the adult brain, thalamus, parietal cortex, pituitary gland, adrenal gland, lymph nodes and lymphoblastic leukemia (Table I).

TABLE I
Distribution of GPR7, GPR8, ppL7 and ppL8 in human central nervous
system and peripheral tissues. DRG, dorsal root ganglia; PBL,
peripheral blood leukocytes

| | ppL7 | GPR7 | ppL8 | GPR8 |
|---|---|---|---|---|
| central nervous system | | | | |
| brain | +++ | ++ | + | ++ |
| fetal brain | +++ | ++ | + | + |
| optic nerves | + | − | − | − |
| parietal cortex | + | − | ++ | ++ |
| hippocampus | ++ | +++ | ++ | +++ |
| caudate nucleus | + | − | − | +++ |
| amygdala | ++ | +++ | ++ | +++ |
| thalamus | + | + | − | ++ |
| hypothalamus | ++ | − | − | − |
| corpus callosum | ++ | + | ++ | + |
| colliculi | + | − | − | − |
| trigeminal nerves | + | − | − | − |
| substantia nigra | +++ | + | +++ | + |
| choroïd plexus | + | − | ++ | − |
| pons | + | − | − | − |
| cerebellum | ++ | − | ++ | + |
| spinal cord | +++ | − | − | − |
| DRG | ++ | − | − | − |
| endocrine system | | | | |
| pituitary gland | − | ++ | − | ++ |
| adrenal gland | + | − | + | ++ |
| thyroid gland | − | − | + | − |
| pancreas | − | − | + | − |
| immune system | | | | |
| spleen | ++ | − | ++ | + |
| fetal spleen | − | + | ++ | + |
| thymus | − | − | − | − |
| PBL | − | + | − | + |
| lymph node | + | − | + | ++ |
| lymphoblastic | − | − | +++ | ++ |
| leukemia | | | | |
| urogenital system | | | | |
| kidney | + | − | + | − |
| fetal kidney | + | + | +++ | + |
| bladder | − | − | − | − |
| prostate | − | ++ | + | − |
| testis | ++ | + | ++ | + |
| uterus | + | − | ++ | − |
| ovary | ++ | − | ++ | − |
| placenta | +++ | − | ++ | − |
| respiratory system | | | | |
| lung | + | + | − | + |
| fetal lung | − | − | ++ | − |
| trachea | + | +++ | +++ | − |
| lung carcinoma | − | + | ++ | + |
| gastro-intestinal tract | | | | |
| stomach | + | − | ++ | − |
| small intestine | + | − | − | − |
| colon | − | + | − | + |
| rectum | − | + | + | + |
| colorectal adenocarcinoma | +++ | − | +++ | − |
| liver | + | − | ++ | − |
| fetal liver | + | − | − | − |
| miscellaneous | | | | |
| heart | + | − | − | − |
| adipose | − | − | − | − |
| skeletal muscle | + | − | + | − |
| skin | + | + | + | + |

### Example 8

Pharmacology of GPR7 and GPR8 Receptors.

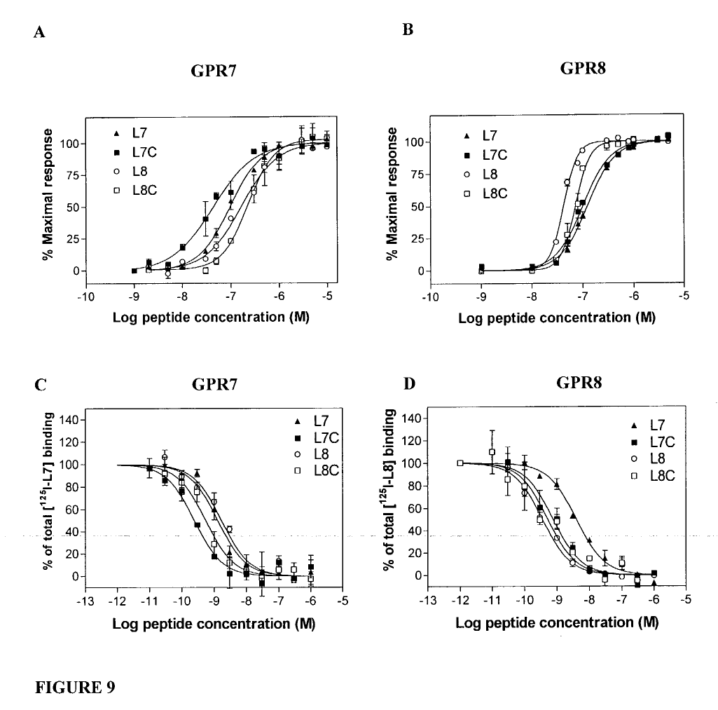

L7, L7C, L8 and L8C were tested in concentration experiments using the aequorin-based functional assay. L7C was the most potent agonist of GPR7 (EC50=50±11 nM, mean±S.E.M.) followed by L7, L8 and L8C (L7: EC50=126±21 nM; L8: EC50=159±12 nM; L8C: EC50=241±10 nM; FIG. 9A). L8 was the most potent agonist of GPR8 (EC50=58±5 nM) followed by L8C, L7C, L7 (L8C: EC50=77±7 nM, L7C: EC50=91±17 nM; L7: EC50=121±16 nM; FIG. 9B). The peptides were inactive on mock-transfected cells up to 10 mM. Human peptide pro-L7 [25-44, WYKPAAGHSSYSVGRAAGLL] derived from the precursor was active in aequorin-based functional assay on GPR7 and GPR8. This peptide was less potent than L7, L7C, L8 and L8C for both receptors (data not shown). Human peptides pro-L7 [51-54, SPYA] and pro-L8 [58-62, SPYLW] derived from the precursor were inactive in aequorin-based functional on GPR7 and GPR8. Other human peptides pro-L7 derived from the precursor could also constitute potential bioactive peptides, possibly on other receptors than GPR7 and GPR8. However, they were not considered up to now. L8 and L7 peptides were iodinated, and binding experiments were performed on membranes obtained from CHO—K1 cells expressing GPR7 or GPR8. In competition binding experiments, L7C and L8C were both more potent than the shorter L7 and L8 on GPR7, L7C being the most active on GPR7. In contrast, L8 was more potent than L8C on GPR8, L8 and L8C being both more potent than L7 and L7C on GPR8.

For GPR7, the IC50 values were of 1.95±0.27 nM, 0.33±0.05 nM, 1.60±0.15 nM and 0.96±0.16 nM for L7, L7C, L8 and L8C respectively, (FIG. 9C), and for GPR8, they were of 4.01±0.13 nM, 0.98±0.28 nM, 0.298±0.002 nM and 0.43±0.15 nM for L7, L7C, L8 and L8C respectively, (FIG. 9D). Opioid compounds that have previously been described as being ligands of GPR7 (O'Dowd B., et al) were tested as well in competition binding assays. However, bremazocin, could not compete with L7 for GPR7 binding up to concentrations of 1 μM (data not shown).

In the experiments described in this section, the aequorin assay measured functional responses by recording the luminescence of aequorin in GPR7- and GPR8-expressing cells following addition of (potential) agonists, as previously described (Kotani, M. et al.). In brief, cells were collected from plates with PBS containing 5 mM EDTA, pelleted, resuspended at 5×106 cells/ml in DMEM-F12 medium containing 0.1% BSA, incubated with 5 μM coelenterazine H (Molecular Probes, Eugene, Oreg.) for 4 h at room temperature, and diluted in DMEM-F12 medium at a concentration of 5×105 cells/ml. Cells where then mixed with the ligands, and the light emission was recorded over 30 s using a Microlumat™ luminometer (Perkin Elmer, Norwalk, Conn.).

In the experiments described in this section, iodinated L8 peptide was obtained using the chloramine T labeling method (Zentech, Belgium). Competition binding assays were performed as described (Kotani, M. et al.) on crude membrane fractions prepared from CHO—K1 cell lines expressing GPR7 or GPR8. Briefly, 1 to 10 μg of membrane proteins were incubated in binding buffer (50 mM HEPES pH 7.4, 5 mM MgCl2, 1 mM CaCl2, 0.5% protease-free BSA) containing 0.1 μM [125I]-L7 or [125I]-L8 radioligand for 90 min at 27° C. Bound tracer was separated by filtration through GF/B filters (Millipore) presoaked in 0.5% polyethylenimine. Filters were then counted by gamma scintillation counting. Results were normalized for total binding in the absence of competitor (100%) and non-specific binding (0%) in the presence of a 100-fold excess of unlabelled ligand, and were analyzed by nonlinear regression, using a single-site competition model (Graph-Pad Prism Software).

### Example 9

Intracellular Coupling of GPR7 and GPR8

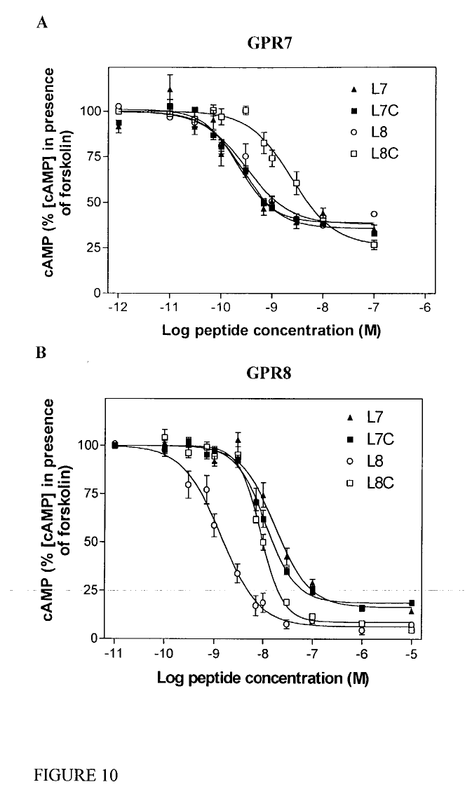

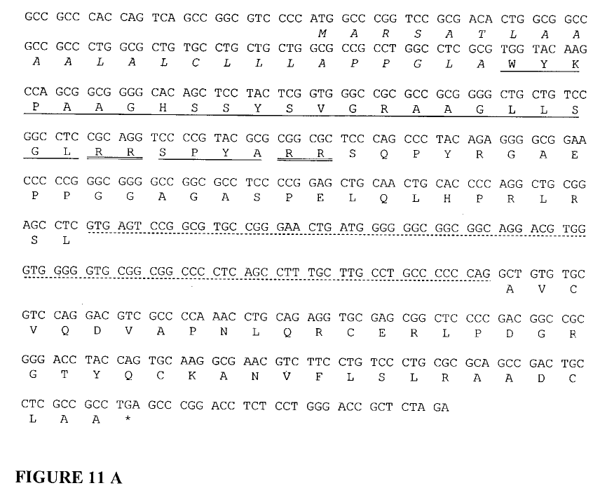

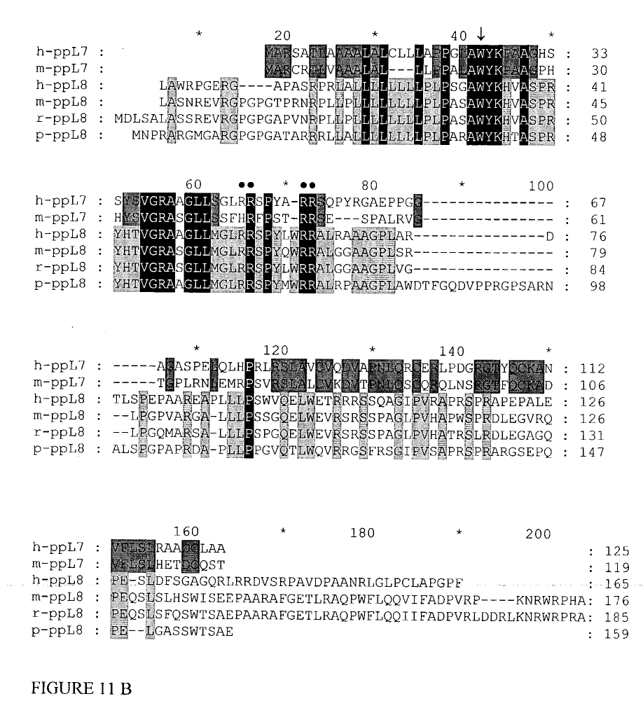

To determine the natural coupling of the receptor to intracellular signaling pathways, stable transfections of GPR7 and GPR8 were produced with CHO—K1 cells. Significant inhibition of the level of cAMP was observed at low concentrations of L7, L7C, L8 or L8C with CHO—K1-GPR7 in the presence of forskolin 5 μM. L7C was slightly more potent (IC50=0.14±0.04 nM) than L7 (0.36±0.05 nM), L8 (IC50=0.42±0.09 nM) and L8C (1.99±0.57 nM, all values as mean±S.E.M.) (FIG. 10A). Significant inhibitions of the levels of cAMP were also observed at low concentrations of L7, L7C, L8 and L8C with CHO—K1-GPR8 in the presence of forskolin 5μM. L8 was more potent (IC50=0.98±0.09 nM) than L8C (IC50=9.8±2.0 nM), L7C (IC50=12.5±2.3 nM) and L7 (IC50=20.9±2.5 nM; all values as mean±S.E.M.) (FIG. 10B). Similar results were obtained with the CHO—WTA11 cells co-expressing Gα16 and GPR7 or GPR8. The effect of L7 and L8 peptides on each receptor was strongly inhibited by pertussis toxin (data not shown). No modification of phosphatidylinositol turnover was observed in COS-7 cells transiently transfected with GPR7 or GPR8. However, Cos-7 cells transfected with GPR7 or GPR8 and with a Gqi5 chimaeric G-protein were positive in their IP3 response with L7 or L8. The cells were challenged with increasing concentrations of the agonists and IP accumulated as a function of the concentrations of L7 for GPR7 (EC50=8.2 nM) and L8 for GPR8 (EC50=15.7 nM, data not shown).

In the experiments in this section, the phosphoinositide accumulation assays comprised Cos-7 cells expressing GPR7 or GPR8, said cells labeled for 12 hours with 3 μCi/ml [3H] inositol in inositol-free DMEM containing 5% FBS. Cells were washed two times with Krebs-Ringer Hepes (KRH) buffer (10 mM Hepes pH 7.4, 146 mM NaCl, 4.2 mM KCl, 0.5 mM MgCl2, 1 mM CaCl2, 55 mM glucose) prior to the incubation with agonists at 37° C. for 30 min in KRH buffer containing 9.4 mM LiCl. The incubation was stopped by replacing the incubation medium with 1 ml of an ice-cold 5% perchloric acid solution. The medium was further neutralized with a 75 mM Hepes, 1.5 M KOH solution. The total inositol phosphate (IP) content was then extracted, and purified on Dowex columns as described (26). Total radioactivity remaining in the membrane fraction was counted after solubilization in 10% Triton, 0.1 N NaOH and used as standard for each well. Results were expressed as the radioactivity associated to IP over the total radioactivity present in membranes.

In the experiments in this section, the cyclic AMP assays comprised CHO—K1 cell lines stably expressing GPR7 or GPR8, said cells cultured in Petri dishes at 37° C. in Ham's F-12 medium, containing or not 100 ng/ml pertussis toxin (PTX). Cells were recovered in PBS containing 5 mM EDTA, resuspended in KRH-IBMX buffer (1.25 mM KH2PO4 pH 7.4, 5 mM KCl, 124 mM NaCl, 1.25 mM MgSO4, 1.45 mM CaCl2, 25 mM Hepes, 0.5 g/l bovine serum albumine, 10 mg/l phenol red, 1 mM IBMX and 13.3 mM glucose) and dispatched into 96-well plates at a density of 2.5×104 cells/well. Cells were further preincubated for 15 min in 1 mM KRH-IBMX buffer and incubated with various concentrations of agonists for 20 min at 37° C., with or without 5 μM forskolin. Incubations were terminated by the addition of lysis buffer (CS1000 kit, Applied Biosystem, USA). The cell lysate was homogenized in the presence of cAMP-AP conjugate and an anti-cAMP-antibody, and cAMP content was quantified by ELISA (CS1000 kit, Applied Biosystem, USA).

### REFERENCES

Altschul et al. (1990) J. Mol. Biol. 403-410

Altschul et al. (1994) Nature Genetics 6: 119-129

Antoch et al. (1997) Cell 89: 655

Ausubel et al. (1995) Short protocols in Molecular Biology, 3^{rd} edition, Jhon Wiley & Sons

Ausubel et al. (1999) supra, pages 7-58 to 7-60

Bader and Ganten (1996) Clinical and Experiemental Pharmacology and Physiology s81-s87

Carmeliet et al. (1996) Nature 380: 435-439

Comb et al. Nature 323: 353-356

Cole et al. (1985) Monoclonal antibodies and Cancer therapy Alan R. Liss Inc. 77-96

Costagliola et al; (2001) J. Clin. Invest. 105: 803-811

Cudermann et al. (1995) Mol. Med. 73: 51-63

Detheux et al. (2000) J. Exp. Med. 192: 1501-1508

Devereux et al. (1984) Nucl. Acid Res. 12: 387

Downes and Gautman (1999) Genomics 62: 544-552

Fink et al. (1988) Proc. Natl. Acad. Sci. USA 85: 6662-6666

Gijon et al. (2000) J. Biol. Chem. 275: 20146-20156

Hafner et al. (2000) Biosens. Bioelectron 15: 149-158

Hammer et al. (1985) Nature 315: 680-683

Haskill et al. (1991) Cell 65: 1281-1289

Hiscott et al. (1993) Mol. Cell. Biol. 13: 6231-6240

Horton and Baxendale (1995) Methods Mol. Biol. 41: 91-105

Ian (1998) Phospholipid signalling protocols. Humana Press

Karin and Altschul (1990) Proc. Natl. Acad. Sci. USA 87: 2264-2268

Karin and Altschul (1993) Proc. Natl. Acad. Sci. USA 90: 5873-5877

Kenimer and Nirenberg (1981) Mol. Pharmacol. 20: 585-591

Kotani, M., et al. (2001) J. Biol. Chem. 276, 34631-34636

Kjelsberg et al. (1992) J. Biol. Chem. 267:1430

Kohler and Milstein (1975) Nature 256: 495-497

Kozbar et al. Immunology today 4: 72

Lee et al. (1987) Nature 325: 386-372

Lee et al. (1987) Cell 49: 741-752

Lee D. K., et al. (1999) Mol. Brain Res. 71; 96-103

Lodish et al. Molecular Cell Biology (Scientific American Books Inc., New York, N.Y., 1995;

Liu et al. (1998) AIDS Res. 14: 1509-1519

Marth (1996) Clin. Invest. 97: 1999

Matsui et al. (1998) J. Immunol. 161: 3469-3473

McWhinney et al. (2000) J. Biol. Chem. 275:2087

Mirzabekov et al. (2000) Nature Biotechnology 18, 649-654

Montminy et al ; (1986) Porc. Natl. Acad. Sci. USA 8: 6682-6686

O'Dowd B., et al. (1995) Genomics 28, 84-91.

Osman et al. (1997) Nature 387: 580

Pan and Mc Ever (1995) J. Biol. Chem. 270: 23077-23083

Parent et al. (1996) J. Biol. Chem. 271:7949

Parma et al. (1993) Nature 365:649

Parma et al. (1998) J. Pharmacol. Exp.Ther. 286: 85

Pinna and Ruzzene (1996) Biochem. Biophys. Acta 1314: 191-225

Reeves (1998) New. Anat. 253: 19

Ren et al. (1993) J. Biol. Chem. 268:16483

Rudolph et al. (1999) J. Biol. Chem. 274: 11824-11831

Salamon et al. (1996) Biophys J. 71: 283-294

Salamon et al. (1999) Trends Biochem. Sci. 24: 213-219

Salamon et al. (2001) Biophys. J. 80: 1557-1567

Samama et al. (1993) J.Biol.Chem 268:4625

Sarrio et al. (2000) Mol. Cell. Biol. 20: 5164-517

Sauer (1998) Methods 14: 381

Schreck and Bacuerle (1990) Mol. Cell. Biol. 10:1281-1286

Shakhov et al; (1990) J; Exp. Med. 171: 35-47

Short et al. (1986) J. Biol. Chem. 261: 9721-9726

Simon, J. et al. (2001) Br. J. Pharmacol. 132, 173-182.

Solomon et al. (1974) Anal. Biochem. 58: 541-548

Stables et al. (1997) Anal. Biochem. 252: 115-126

Traynor and Nahorski (1995) Mol. Pharmacol. 47: 848-854

Verma et al. (1987) Cell 51: 513-514

Wagner et al. (1997) Nucleic Acids Res. 25: 4323

White and Yannoutsos Current topics in complement research 64^{th} Forum in Immunology 88-94

1. A polypeptide ligand comprising the consensus sequence WYKxxAxxxxxT/SVGRAAGLLxGL that binds to a GPR7 polypeptide.

2. The polypeptide ligand of claim 1 that comprises a sequence selected from SEQ ID NO: 3, SEQ ID NO: 5, SEQ ID NO: 7, SEQ ID NO: 9, SEQ ID NO: 11, SEQ ID NO: 15, SEQ ID NO: 17, SEQ ID NO: 19, SEQ ID NO: 21, SEQ ID NO: 25 and SEQ ID NO: 27.

3. The polypeptide ligand of claim 1 that comprises a sequence selected from SEQ ID NO: 7, SEQ ID NO: 11, SEQ ID NO: 15, SEQ ID NO: 17, SEQ ID NO: 21.

4. A method of identifying an agent that modulates the function of a G-protein coupled receptor 7 (GPR7), said method comprising:
a) contacting a GPR7 polypeptide with a polypeptide ligand of claim 1 in the presence and absence of a candidate modulator under conditions permitting the binding of said ligand to said GPR7 polypeptide; and
b) measuring the binding of said GPR7 polypeptide to said ligand, wherein a decrease in binding in the presence of said candidate modulator, relative to the binding in the absence of said candidate modulator, identifies said candidate modulator as an agent that modulates the function of GPR7.

5. A method of detecting the presence in a sample of an agent in a sample that modulates the function of GPR7, said method comprising:
a) contacting a GPR7 polypeptide with a polypeptide ligand of claim 1 in the presence and absence of said sample under conditions permitting the binding of said ligand to said GPR7 polypeptide; and
b) measuring the binding of said GPR7 polypeptide to said ligand, wherein a decrease in binding in the presence of said sample, relative to the binding in the absence of said candidate modulator, identifies said candidate modulator as an agent that modulates the function of GPR7.

6. A method of identifying an agent that modulates the function of GPR7, said method comprising:
a) contacting a GPR7 polypeptide with a polypeptide ligand of claim 1 in the presence and absence of a candidate modulator; and
b) measuring a signalling activity of said GPR7 polypeptide, wherein a change in the activity in the presence of said candidate modulator relative to the activity in the absence of said candidate modulator identifies said candidate modulator as an agent that modulates the function of GPR7.

7. A method of identifying an agent that modulates the function of GPR7, said method comprising:
a) contacting a GPR7 polypeptide with a candidate modulator;
b) measuring a signalling activity of said GPR7 polypeptide in the presence of said candidate modulator; and
c) comparing said activity measured in the presence of said candidate modulator to said activity measured in a sample in which said GPR7 polypeptide is contacted with a polypeptide ligand of claim 1 at its EC₅₀, wherein said candidate modulator is identified as an agent that modulates the function of GPR7 when the amount of said activity measured in the presence of said candidate modulator is at least 20% of the amount induced by said ligand present at its EC₅₀.

8. A method of detecting the presence, in a sample, of an agent that modulates the function of GPR7, said method comprising:
a) contacting a GPR7 polypeptide with a polypeptide ligand of claim 1 in the presence and absence of said sample;
b) measuring a signalling activity of said GPR7 polypeptide; and
c) comparing the amount of said activity measured in a reaction containing said GPR7 polypeptide and said ligand without said sample to the amount of said activity measured in a reaction containing said GPR7 polypeptide, said ligand and said sample, wherein a change in said activity in the presence of said sample relative to the activity in the absence of said sample indicates the presence, in said sample, of an agent that modulates the function of GPR7.

9. A method of detecting the presence, in a sample, of an agent that modulates the function of GPR7, said method comprising:
a) contacting a GPR7 polypeptide with said sample;
b) measuring a signalling activity of said GPR7 polypeptide in the presence of said sample; and
c) comparing said activity measured in the presence of said sample to said activity measured in a reaction in which said GPR7 polypeptide is contacted with a polypeptide ligand of claim 1 present at its EC₅₀, wherein an agent that modulates the function of GPR7 is detected if the amount of said activity measured in the presence of said sample is at least 20% of the amount induced by said ligand present at its EC₅₀.

10. The method according to claim 4 or claim 5 wherein said ligand is detectably labelled.

11. The method of claim 10 wherein said label is a moiety selected from the group consisting of a radioisotope, a fluorophore, a quencher of fluorescence, an enzyme.

12. The method according to claim 4 wherein said contacting is performed in or on a cell expressing said GPR7 polypeptide.

13. The method according to claim 4 wherein said contacting is performed in or on synthetic liposomes.

14. The method according to claim 4 wherein said contacting is performed in or on virus-induced budding membranes containing a GPR7 polypeptide.

15. The method according to claim 4 wherein said GPR7 polypeptide is expressed by cells and is present in a membrane fraction of said cells.

16. The method according to claim 4 wherein said measuring is performed using a method selected from label displacement, surface plasmon resonance, fluorescence resonance energy transfer, fluorescence quenching, and fluorescence polarization.

17. The method according to claim 4 wherein said agent is selected from the group consisting of a natural or synthetic peptide, a polypeptide, an antibody or antigen-binding fragment thereof, a lipid, a carbohydrate, a nucleic acid, a peptide-nucleic acid, and a small organic molecule.

18. The method according to claim 6 wherein said step of measuring a signalling activity of said GPR7 polypeptide comprises detecting a change in the level of a second messenger.

19. The method according to claim 18 wherein said measuring comprises measurement of guanine nucleotide binding or exchange, adenylate cyclase activity, cAMP, Protein Kinase C activity, phosphatidylinositol breakdown, diacylglycerol, inositol triphosphate, intracellular calcium, MAP kinase activity, tyrosine kinase activity, or reporter gene expression.

20. The method according to claim 18 wherein said measuring comprises using an aequorin-based assay.

21. A kit for screening for agents that modulate the binding of a polypeptide ligand to GPR7, said kit comprising an isolated polypeptide ligand of claim 1.

22. A kit for screening for agents that modulate the signalling activity of GPR7, said kit comprising an isolated polypeptide ligand of claim 1.

23. The kit according to either of claims 21 and 22 further comprising an isolated GPR7 polypeptide.

24. The kit according to either of claims 21 and 22 further comprising an isolated polynucleotide encoding a GPR7 polypeptide.

25. The kit according to either of claims 21 and 22 further comprising cells transformed with a polynucleotide encoding a GPR7 polypeptide.

26. The kit according to either of claims 21 and 22 comprising a high-throughput screening kit format.

27. A method of diagnosing a disease or disorder characterized by dysregulation of GPR7 signalling, said method comprising:
a) contacting a tissue sample with an antibody specific for a GPR7 polypeptide, said GPR7 polypeptide capable of associating with a polypeptide ligand of claim 1;
b) detecting binding of said antibody to said tissue sample; and
c) comparing the binding detected in step (b) with a standard, wherein a difference in binding relative to said standard is diagnostic of a disease or disorder characterized by dysregulation of GPR7.

28. A method of diagnosing a disease or disorder characterized by dysregulation of GPR7 signalling, said method comprising:
a) isolating nucleic acid from a tissue sample;
b) amplifying a GPR7 polynucleotide encoding a portion of a GPR7 polypeptide, said GPR7 polypeptide capable of associating with a polypeptide ligand of claim 1, said amplification using said nucleic acid as a template; and
c) comparing the amount of amplified GPR7 polynucleotide produced in step (b) with a standard, wherein a difference in said amount of amplified GPR7 polynucleotide relative to said standard is diagnostic of a disease or disorder characterized by dysregulation of GPR7.

29. A method of diagnosing a disease or disorder characterized by dysregulation of GPR7 signalling, said method comprising:
a) isolating nucleic acid from a tissue sample;
b) amplifying a GPR7 polynucleotide encoding a portion of a GPR7 polypeptide, said GPR7 polypeptide capable of associating with a polypeptide ligand of claim 1, said amplification using said nucleic acid as a template; and
c) comparing the sequence of said amplified GPR7 polynucleotide produced in step (b) with a standard, wherein a difference in said sequence, relative to said standard is diagnostic of a disease or disorder characterized by dysregulation of GPR7.

30. The method of claim 29 wherein said standard is a nucleic acid of SEQ ID NO: 24.

31. The method of claim 29 wherein said comparing the sequence is performed on a microarray.

32. A kit for the diagnosis or a disease or disorder characterized by dysregulation of GPR7 signalling, comprising an antibody specific for a GPR7 polypeptide.

33. A kit for the diagnosis of a disease or disorder characterized by dysregulation of GPR7 signalling comprising an isolated GPR7 Polypeptide.

34. A kit for the diagnosis of a disease or disorder characterized by dysregulation of GPR7 signalling comprising a polypeptide ligand of claim 1.

35. The kit according to claim 32 or 33 comprising a polypeptide ligand of claim 1.

36. The kit according to claim 32 or 34, comprising an isolated polynucleotide encoding a GPR7 polypeptide.

37. The kit according to claim 32 or 34, comprising a cell transformed with a polynucleotide encoding a GPR7 polypeptide.

38. The method according to claim 1 wherein said GPR7 polypeptide comprises the sequence of SEQ ID NO: 23.

39. The kit according to any one of claims 33, 35; 36 and 37 wherein said GPR7 polypeptide comprises the sequence of SEQ ID NO: 23.

40. A polypeptide which comprises an immunologically active fragment of a polypeptide according to claim 1.

41. An isolated nucleic acid capable of encoding a polypeptide of claim 1.

42. A nucleic acid comprising a sequence of SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 26, or SEQ ID NO: 28.

43. A nucleic acid of at least 15 nucleotides in length capable of specifically hybridising with the nucleic acid of claim 41.

44. A nucleic acid molecule of at least 15 nucleotides in length, capable of specifically amplifying the nucleic acid of claim 41.

45. A vector comprising a nucleic acid sequence according to claim 41.

46. The vector of claim 45 wherein said vector is an expression vector wherein the nucleic acid sequence is operably linked to one or more control sequences allowing the expression of said sequence in prokaryotic and/or eukaryotic host cells.

47. The vector of claim 45, wherein said vector is an adenoviral vector.

48. A host cell comprising an integrated or episomal copy of a nucleic acid molecule of claim 41 or a vector of claim 45.

49. An antibody specifically recognising a polypeptide according to claim 1.

50. An antibody specifically recognising a nucleic acid according to claim 41.

51. A method of inhibiting the activity of a polypeptide of claim 1, the method comprising contacting said polypeptide with an antibody of claim 49.

52. A method of detecting a polypeptide of claim 1, the method comprising contacting a sample to be tested for said polypeptide with an antibody of claim 49.

53. A method of detecting a nucleic acid according to claim 41, the method comprising contacting a sample to be tested for said nucleic acid with a nucleic acid of claim 43.

54. A method for producing a polypeptide according claim 1 comprising:
(a) culturing host cells comprising a nucleic acid according to claim 41, under conditions allowing the expression of the polypeptide, and,
(b) recovering the produced polypeptide from the culture.

55. A composition comprising a polypeptide of claim 1 in admixture with a pharmaceutically acceptable carrier.

56. A composition comprising a nucleic acid of claim 41 or 43 in admixture with a pharmaceutically acceptable carrier.

57. A composition comprising a antibody of claim 49 in admixture with a pharmaceutically acceptable carrier.

58. A method for treating or preventing a disease involving GPR7 receptor dysfunction, comprising administering a polypeptide selected from the group consisting of SEQ ID NO: 3, SEQ ID NO: 5, SEQ ID NO: 7, SEQ ID NO: 9, SEQ ID NO: 11, SEQ ID NO: 15, SEQ ID NO: 17, SEQ ID NO: 19, SEQ ID NO: 21, SEQ ID NO: 25, and SEQ ID NO: 27.

59. A method for treating or preventing a disease involving GPR7 receptor dysfunction, comprising administering a polynucleotide encoding a polypeptide selected from the group consisting of SEQ ID NO: 3, SEQ ID NO: 5, SEQ ID NO: 7, SEQ ID NO: 9, SEQ ID NO: 11, SEQ ID NO: 15, SEQ ID NO: 17, SEQ ID NO: 19, SEQ ID NO: 21, SEQ ID NO: 25, or SEQ ID NO: 27.

60. The method of claim 59 wherein said polynucleotide comprises a sequence selected from the group consisting of SEQ ID NO: 4, SEQ ID NO: 6, SEQ ID NO: 8, SEQ ID NO: 10, SEQ ID NO: 12, SEQ ID NO: 16, SEQ ID NO: 18, SEQ ID NO: 20, SEQ ID NO: 22, SEQ ID NO: 26, and SEQ ID NO: 28.

61. A method of treating or preventing a disease involving GPR7 receptor dysfunction, comprising administering an antibody of claim 49 or 50.

62. A method of preparing a medicament for the prevention or treatment of a disease caused by GPR7 dysfunction, comprising forming an admixture of a polypeptide of claim 1 and a pharmaceutically acceptable carrier.

63. A transgenic non-human animal comprising one or more copies of a nucleic acid of claim 41 stably integrated into the genome of said animal.

64. The transgenic non-human animal of claim 63 wherein said one or more copies of a nucleic acid are operably linked to regulatory elements that modulate the expression of said nucleic acid.

65. A non-human animal comprising a deletion of one or more exons from a genomic sequene encoding a nucleic acid of claim 41.

66. A non-human animal comprising a targeted mutation in a genomic sequence corresponding to a nucleic acid of claim 41.

67. A non-human animal comprising a targeted mutation in a regulatory sequence for a genomic sequence corresponding to a nucleic acid of claim 41.
